(19) United States
(12) Patent Application Publication
(10) Pub. No.: US 2026/0201350 A1
(43) Pub. Date: Jul. 16, 2026

(54) MRNA RECOMBINANT CAPPING ENZYMES

(71) Applicant: SANOFI PASTEUR INC., Swiftwater, PA (US)

(72) Inventors: Jianping CUI, Cambridge, MA (US); Yaroslav MOROZOV, Cambridge, MA (US)

(21) Appl. No.: 19/138,543

(22) PCT Filed: Dec. 15, 2023
(86) PCT No.: PCT/EP2023/086197

§ 371 Date: Jun. 12, 2025

(30) Foreign Application Priority Data
Dec. 15, 2022 (EP) 22306892.5

(51) Int. Cl. C12N 9/10; C07K 14/00; C12N 9/12; C12N 15/52; C12N 15/62; C12N 15/63; C12P 19/34

(52) U.S. Cl. CPC C12N 9/1007; C07K 14/00; C12N 9/1241; C12N 15/52; C12N 15/62; C12N 15/63; C12P 19/34; C12Y 201/01057; C07K 2319/00

(57) ABSTRACT

Provided herein is a fusion protein comprising a messenger RNA (mRNA) capping enzyme polypeptide linked to a Fh8 polypeptide or fragment thereof. Also provided are methods of preparing a mRNA comprising the step of capping using the capping enzymes described herein.

### RELATED APPLICATIONS

This application is a 35 U.S.C. § 371 filing of International Patent Application No. PCT/EP2023/086197, filed Dec. 15, 2023, which claims the benefit of European Patent Application No. 22306892.5, filed Dec. 15, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jun. 11, 2025, is named 766811_SA9-333US_ST26.xml and is 90,127 bytes in size.

### BACKGROUND OF THE DISCLOSURE

The 5′ terminal m7G cap present on most eukaryotic mRNAs promotes translation at the initiation level. For most mRNAs, elimination of the cap structure causes a loss of stability, especially against exonuclease degradation, and a decrease in the formation of the initiation complex of mRNAs for protein synthesis.

Vaccinia capping enzymes, D1-D12 and VP39, are commercially available and widely used for enzymatic capping in mRNA manufacturing processes which use post-transcriptional in vitro capping.

The vaccinia RNA-capping system is comprised of a multifunctional mRNA cap-synthesizing enzyme (D1 and D12 subunits) containing three catalytic domains called triphosphatase (TPase), guanylyltransferase (GTase), and N7 methyltransferase (N7MTase). The 5′-triphosphate of the nascent mRNA is first hydrolyzed by the TPase to yield 5′-diphosphate RNA, which is then sequentially transferred to other internal domains to be capped and methylated, the latter reaction with allosteric stimulation through direct association with D12. The sequential reactions lead to the formation of Cap-0, characteristic of a guanine added to the 5′-end with a head-to-head triphosphate group. Cap assembly is completed by the viral VP39, a bifunctional protein that catalyzes the methyl group to the ribose 02′ of the penultimate nucleotide, forming Cap-1.

Current protein production procedures for the generation of capping enzymes like D1, D12, and VP39 enzymes to be used for enzymatic capping in the mRNA manufacturing process suffer from low solubility yields. There remains a need for more effective reagents and methods for the large-scale production of mRNA capping enzymes.

### SUMMARY OF THE DISCLOSURE

The present disclosure provides a fusion protein comprising a messenger RNA (mRNA) capping enzyme polypeptide linked to a Fh8 polypeptide or fragment thereof.

In some embodiments, the Fh8 polypeptide or fragment thereof comprises an amino acid sequence comprising at least 90% identity to an amino acid sequence set forth in SEQ ID NO: 10.

In some embodiments, the Fh8 polypeptide or fragment thereof is linked to the N-terminus or the C-terminus of the capping enzyme polypeptide.

In some embodiments, the capping enzyme polypeptide comprises a vaccina virus D1 subunit.

In some embodiments, the vaccina virus D1 subunit comprises an amino acid sequence comprising at least 90% identity to an amino acid sequence set forth in SEQ ID NO: 1; and/or the fusion protein comprises an amino acid sequence comprising at least 90% identity to an amino acid sequence set forth in SEQ ID NO: 3.

In some embodiments, the capping enzyme polypeptide comprises a vaccina virus D12 subunit.

In some embodiments, the vaccina virus D12 subunit comprises an amino acid sequence comprising at least 90% identity to an amino acid sequence set forth in SEQ ID NO: 2.

In some embodiments, the capping enzyme polypeptide comprises a vaccina virus VP39 polypeptide or fragment thereof.

In some embodiments, the VP39 polypeptide comprises an amino acid sequence comprising at least 90% identity to an amino acid sequence set forth in SEQ ID NO: 6, and/or the fusion protein comprises an amino acid sequence comprising at least 90% identity to an amino acid sequence set forth in SEQ ID NO: 4.

In some embodiments, the VP39 polypeptide fragment comprises an amino acid sequence comprising at least 90% identity to an amino acid sequence set forth in SEQ ID NO: 7, and/or the fusion protein comprises an amino acid sequence comprising at least 90% identity to an amino acid sequence set forth in SEQ ID NO: 5.

In some embodiments, the capping enzyme polypeptide comprises a bluetongue virus VP4 polypeptide or fragment thereof.

In some embodiments, the VP4 polypeptide comprises an amino acid sequence comprising at least 90% identity to an amino acid sequence set forth in SEQ ID NO: 16; and/or the fusion protein comprises an amino acid sequence comprising at least 90% identity to an amino acid sequence set forth in SEQ ID NO: 17, SEQ ID NO: 18, or SEQ ID NO: 22.

In one aspect, the disclosure provides a polynucleotide comprising a nucleotide sequence that encodes the fusion protein described above.

In some embodiments, the nucleotide sequence is codon optimized.

In some embodiments, the nucleotide sequence comprises at least 90% identity to a nucleotide sequence set forth in SEQ ID NO: 23, SEQ ID NO: 24, SEQ ID NO: 32, SEQ ID NO: 33, or SEQ ID NO: 37.

In one aspect, the disclosure provides an expression vector comprising the polynucleotide described herein.

In one aspect, the disclosure provides a host cell comprising the expression vector described above.

In some embodiments, the host cell is an E. coli cell.

In some embodiments, the E. coli cell is a BL21 (DE3) or Origami E. coli cell strain.

In one aspect, the disclosure provides a method of expressing a fusion protein, comprising culturing the host cell described under conditions sufficient to express the fusion protein.

In some embodiments, the fusion protein is further isolated from the host cell.

In one aspect, the disclosure provides a method of capping an mRNA, comprising incubating the mRNA with the fusion protein described under conditions sufficient to cap the mRNA with a cap0 structure.

In one aspect, the disclosure provides a method of converting a cap0 structure on an mRNA to a cap1 structure, comprising incubating the mRNA with the fusion protein described under conditions sufficient to cap the mRNA with a cap1 structure.

In one aspect, the disclosure provides a method of capping an mRNA, comprising incubating the mRNA with the fusion protein described under conditions sufficient to cap the mRNA with a cap1 structure.

In one aspect, the disclosure provides a process of preparing an mRNA comprising a step of capping, comprising: a) incubating the mRNA with the fusion protein described above under conditions sufficient for the mRNA to be capped, b) optionally purifying the capped mRNA, c) optionally tailing the mRNA with a polyadenylation step, and d) optionally purifying the capped polyadenylated mRNA.

In one aspect, the disclosure provides a capped mRNA obtained by the method described above, or by the process described above.

### BRIEF DESCRIPTION OF THE DRAWINGS

FIG. 1 depicts the vaccinia RNA-capping system comprised of a multifunctional mRNA cap-synthesizing enzyme (D1 and D12 subunits) containing three catalytic domains called triphosphatase (TPase), guanylyltransferase (GTase), and N7 methyltransferase (N7MTase). The 5′-triphosphate of the nascent mRNA is first hydrolyzed by the TPase to yield 5′-diphosphate RNA, which is then sequentially transferred to other internal domains to be capped and methylated, the latter reaction with allosteric stimulation through direct association with D12. The sequential reactions lead to the formation of Cap-0, characteristic of a guanine added to the 5′-end with a head-to-head triphosphate group. Cap assembly is completed by the viral VP39, a bifunctional protein that catalyzes the methyl group to the ribose 02′ of the penultimate nucleotide, forming Cap-1.

FIG. 2A-FIG. 2D depict the pET-28 plasmid maps designed for expression of D1 and D12 in E. coli. FIG. 2A is a map of the control plasmid which does not contain a solubility tag. FIG. 2B is a map of the experimental plasmid in which D1 has a N-terminal SUMO solubility tag. FIG. 2C is a map of the experimental plasmid in which D1 has a N-terminal Fh8 solubility tag. FIG. 2D is a map of the experimental plasmid in which D1 has a N-terminal periplasmic targeting tag, phoA.

FIG. 3A-FIG. 3C depict the pET-28 based plasmid maps designed for expression of VP39 in E. coli. FIG. 3A is a map of a plasmid in which VP39 has a N-terminal GST tag. FIG. 3B is a map of a plasmid in which VP39-C26 has a N-terminal GST tag. FIG. 3C is a map of a plasmid in which VP39 has a N-terminal Fh8 tag.

FIG. 4A-FIG. 4B are bar graphs which detail the soluble expression pattern of the D1-D12 expression plasmids with different solubility tags tested in E. coli host strain Artic Express (FIG. 4A) or BL21 (DE3) (FIG. 4B).

FIG. 5 is a table summarizing the soluble expression pattern of the D1-D12 constructs with different solubility tags tested in E. coli host strains ArcticExpress, Shuffle, BL21 (DE3), Origami. A white box indicates that there was undetectable soluble expression (no band seen on the gel), a gray box indicates that there was low soluble expression (faint but visible band on the gel), and a black box indicates that there is high soluble expression (strong clear band present on the gel).

FIG. 6 is an image of a western blot JESS gel comparing the soluble protein expression levels of E. Coli BL21 cells which were transformed with pET-28 plasmid containing His-Fh8-D1-D12 (lane 2) or pET-28 plasmid containing His-D1-D12 (lane 3). Lane 1 contains the protein ladder.

FIG. 7A-FIG. 7C display the results of optimizing the expression induction conditions for enhanced soluble enzyme yield for the D1-D12 plasmid containing the Fh8-tagged D1 subunit transformed into E. coli BL21 (DE3) cells. FIG. 7A is a western blot JESS gel image showing the soluble and total expression of Fh8-tagged D1 subunit under the following conditions: no IPTG induction or IPTG induction at OD₆₀₀ 0.1-0.4. FIG. 7B is the quantification of the JESS gel image normalized to the protein concentration in the sample and FIG. 7C is its graphical representation.

FIG. 8A-FIG. 8B display the results of the activity of Fh8-tagged D1-D12 enzyme in a capping reaction with an RNA substrate. FIG. 8A is an image of a dot blot containing the RNA substrate which was co-incubated with escalating concentrations of either a commercially available D1-D12 from New England Biolabs (NEB) or the Fh8 tagged D1-D12 enzyme for 0, 10, 20, or 30 minutes and detected with an anti-7 mG cap antibody. FIG. 8B is a graph comparing the average reaction velocity (ng/min) per concentration (ng/ml) of either the commercially available D1-D12 or the Fh8 tagged D1-D12 enzyme.

FIG. 9 is a bar graph displaying the yield of soluble protein expression for E. coli strains Arctic Express, Shuffle, BL21, Origami, or C41 transformed with the plasmid containing either the His6-GST tagged VP39, the His6-GST tagged VP39-C26, the His6-Fh8 tagged VP39 or the His6-Fh8 tagged VP39-C26 construct after IPTG induction grown in a BioFlo fermentation system. All constructs were codon optimized either by method A or by method B indicated on each X-axis construct label by a terminal notation of “A” or “B”.

FIG. 10A-FIG. 10B display the results of soluble expression of E. coli BL21 (DE3) cells transformed with a plasmid containing the Fh8 tagged-VP39 C26 and grown in a fermenter. FIG. 10A is a western blot JESS gel image of samples E. coli BL21 (DE3) cells transformed with a plasmid containing Fh8 tagged-VP39 C26 before and after IPTG induction (0.1 mM IPTG) at OD₆₀₀ 0.4 and 22° C. FIG. 10B is a table calculating the amount of soluble VP39 enzyme using GST-tagged VP39 as a standard. The standard was used at 0.025-0.2 mg/mL (see lanes 6 to 9).

FIG. 11 displays the results of the O-methyltransferase (OMT) activity of Fh8-VP39-C26 on a cap-0 RNA substrate. The MTase-Glo Assay from Promega was employed. The tested O-methyltransferases use SAM as a methyl donor to methylate the target substrate, thereby leading to SAH production. A MTase-Glo™ reagent is added to convert SAH to ADP. MTase-Glo™ Detection Solution is then added to convert ADP to ATP, which is detected via a luciferase reaction creating detectable luminescence. Various amounts of each enzyme were employed with fixed amount of substrate and fixed reaction time. SAH is a surrogate for cap-1 (1:1 stoichiometry). The results from the O-methyltransferase (OMT) activity assay plotted in a graph comparing the enzyme velocity (expressed as the amount of S-adenosyl homocysteine, SAH, produced per hour) per concentration (pmol) for either the commercially available VP39 (OMT NEB) or the Fh8 tagged Fh8-VP39-C26.

FIG. 12A-FIG. 12B display the soluble expression pattern of VP4 from E. coli BL21 (DE3) cells transformed with plasmid containing VP4 solubility tagged constructs. FIG. 12A is a bar graph representation of VP4 expression and FIG. 12B is the quantification of the western blot Jess gel image normalized to the protein concentration in the same.

### DETAILED DESCRIPTION OF THE DISCLOSURE

The present disclosure is directed to a fusion protein comprising a messenger RNA (mRNA) capping enzyme polypeptide linked to a Fh8 polypeptide or fragment thereof. Also provided are methods of preparing a mRNA comprising a step of capping using the capping enzymes described herein.

### Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence”, is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising”, otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by +10%, +5%, +4%, +3%, +2%, +1%, +0.9%, +0.8%, +0.7%, +0.6%, +0.5%, +0.4%, +0.3%, +0.2%, +0.1%, +0.05%, or +0.01%. In some embodiments, “about” indicates deviation from the indicated numerical value by +10%. In some embodiments, “about” indicates deviation from the indicated numerical value by +5%. In some embodiments, “about” indicates deviation from the indicated numerical value by +4%. In some embodiments, “about” indicates deviation from the indicated numerical value by +3%. In some embodiments, “about” indicates deviation from the indicated numerical value by +2%. In some embodiments, “about” indicates deviation from the indicated numerical value by +1%. In some embodiments, “about” indicates deviation from the indicated numerical value by +0.9%. In some embodiments, “about” indicates deviation from the indicated numerical value by +0.8%. In some embodiments, “about” indicates deviation from the indicated numerical value by +0.7%. In some embodiments, “about” indicates deviation from the indicated numerical value by +0.6%. In some embodiments, “about” indicates deviation from the indicated numerical value by +0.5%. In some embodiments, “about” indicates deviation from the indicated numerical value by +0.4%. In some embodiments, “about” indicates deviation from the indicated numerical value by +0.3%. In some embodiments, “about” indicates deviation from the indicated numerical value by +0.1%. In some embodiments, “about” indicates deviation from the indicated numerical value by +0.05%. In some embodiments, “about” indicates deviation from the indicated numerical value by +0.01%.

The polynucleotides according to the present disclosure may be codon optimized. “Codon optimization” or “codon optimized” means that the sequence of the polynucleotides is optimized for codon usage of the host organism (for instance, E. coli). The genetic code has 64 possible codons. Each codon comprises a sequence of three nucleotides. Codons that encode the same amino acid are called synonymous codons. During protein synthesis, a species or a gene typically prefers to use one or several specific synonymous codons called optimal codons, and this phenomenon is known as codon usage bias. A codon usage table contains experimentally derived data regarding how often, for the particular host organism (for instance E. coli) from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid. Codon usage tables are stored in publicly available databases, such as the Codon Usage Database (Nakamura et al. (2000) Nucleic Acids Research 28 (1), 292; available online at https://www.kazusa.or.jp/codon/). The expression levels of proteins are highly correlated with codon usage bias of the host organism. Codon optimization involves increasing the host organism optimal codon content in the polynucleotide sequence without changing the sequence of the amino acid to promote expression of the recombinant gene in the host organism. Any codon optimization method may be used to generate the codon optimized polynucleotides of the disclosure and such kind of methods are known to the person skilled in the art (see for instance methods described in Al-Hawash et al. (2017), Gene Reports, Vol. 9, 46-53).

As used herein, the term “messenger RNA” or “mRNA” refers to a polynucleotide that encodes at least one polypeptide. mRNA as used herein encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a polyA tail. mRNA can be purified from natural sources, produced using recombinant expression systems (e.g., in vitro transcription) and optionally purified, or chemically synthesized.

Also included in the present disclosure are fragments or variants of polypeptides, and any combination thereof. The term “fragment” or “variant” when referring to the polypeptides of the present disclosure include any polypeptides which retain at least some of the properties (e.g., the enzymatic activity or the solubilization activity) of the reference polypeptide. Fragments of polypeptides include C-terminal fragments and N-terminal fragments, as well as deletion fragments but do not include the naturally occurring full-length polypeptide (or mature polypeptide). Variants of the polypeptides of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions. In some embodiments, a fragment has a length of at least 20 amino acids, at least 30 amino acids, at least 40 amino acids, at least 50 amino acids, or at least 60 amino acids. The enzymatic activity of a fragment may be assessed by any method well known by the skilled person, such as a Dot Blot assay and, depending on the enzymatic activity being evaluated, GTP-PPi exchange assay, inorganic pyrophosphatase assay, RNA triphosphatase assay, methyltransferase assay (for example a MTase Glo Methyltransferase assay), or guanylyltransferase assay.

A “capping enzyme” is one or more polypeptides having enzymatic activities that, in the presence of suitable reaction conditions, catalyze the attachment of the 5′ cap to messenger RNA molecules, resulting in synthesis of capped RNA, including RNA having a cap 0 structure or a cap 1 structure. In general, a capping enzyme comprises RNA triphosphatase and RNA guanylyltransferase enzymatic activities, and optionally, the capping enzyme can also comprise RNA guanine-7-methyltransferase enzymatic activity. Without limiting the disclosure, vaccinia virus capping enzyme and the bluetongue virus VP4 capping enzyme having these enzymatic activities, including both full-length and enzymatically active portions thereof, which have been identified, purified, characterized, cloned, and expressed from a clone are examples of capping enzymes (Moss et al. (1991), 266 (3): 1355-1358; J Biol Chem. Sutton et al. (2007), Nat Struct Mol Biol. 14 (5): 449-451). As used herein, “capping enzyme” is interchangeable with terms “cap-synthesizing enzyme”.

A “fusion protein” is a protein created through the joining of two or more genes that originally coded for separate proteins or polypeptides. This typically involves removing the stop codon from a DNA sequence coding for the first protein, then appending the DNA sequence of the second protein in frame through ligation or overlap extension PCR. If more than two genes are fused, the other genes are added in frame in the same manner. The resulting DNA sequence will then be expressed by a cell as a single protein. In the context of the present disclosure, the fusion protein can be engineered to include the full sequence of the first and/or second protein, or only a fragment of the first and/or second protein (e.g., a capping enzyme polypeptide or fragment thereof linked to a Fh8 polypeptide or fragment thereof). The joining of the two or more genes may be made in any order. The first amino acid or nucleotide sequence can be directly joined or juxtaposed to the second amino acid or nucleotide sequence or alternatively an intervening sequence can covalently join the first sequence to the second sequence. In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains).

The term “linked” or “attached” or “fused” as used herein refers to a first amino acid sequence or nucleotide sequence covalently or non-covalently joined to at least a second amino acid sequence or nucleotide sequence, respectively, thereby producing a fusion protein. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively. The term “linked” is also indicated by a hyphen (-).

The disclosure describes nucleic acid sequences (e.g., DNA and RNA sequences) and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 85%, at least about 90%, at least about 95%, at least about 96%, at least about 97%, at least about 98%, at least about 99%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

D1-D12 mRNA Capping Enzyme

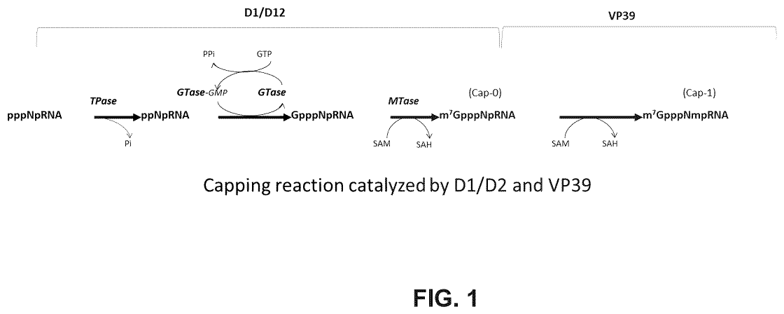

The Vaccinia virus Capping Enzyme (VCE) facilitates the addition of 7-methylguanylate cap structures (Cap-0) to the 5′end of RNA (Shuman, S. (1990). J. Biol. Chem. 265, 11960-11966). Vaccinia capping enzyme is composed of two subunits (D1 and D12). Minimally, to perform mRNA capping, the system requires the heterodimer that includes a large subunit D1 (about 97 kDa) and a small subunit D12 (about 33 kDa). The three enzymatic functions include phosphatase activity (cleavage of the nascent 5′ triphosphate of mRNA to a diphosphate), guanylyl transferase activity (incorporation of a GTP molecule to the 5′ end of the mRNA moiety) and methylation activity (incorporation of a methyl group at the N7 position of the guanylyl base). This process is shown in FIG. 1 and is known as mRNA capping.

The 5′-triphosphate of the nascent mRNA is first hydrolyzed by the TPase to yield 5′-diphosphate RNA, which is then sequentially transferred to other internal domains to be capped and methylated, the latter reaction with allosteric stimulation through direct association with D12. The sequential reactions lead to the formation of Cap-0, characteristic of a guanine added to the 5′-end with a head-to-head triphosphate group. Cap assembly is completed by the viral VP39, a bifunctional protein that catalyzes the methyl group to the ribose 02′ of the penultimate nucleotide, forming Cap-1.

Previously, an expression plasmid carrying a His6 tagged D1-D12 was described for the purification of the vaccinia virus capping enzyme. Fuchs et al. (2016), RNA, vol. 22 (9): 1454-1466. However, the amount of enzyme needed to produce capped RNA and the efficiency of the protein purification process needs to be improved to suit large scale production methods required for the manufacturing of mRNA-based therapeutics.

As used herein, “D1-D12 mRNA capping enzyme” is interchangeable with terms, “vaccinia capping enzyme”, “vaccinia capping complex”, or “D1-D12 complex”.

The fusion proteins described herein may comprise one or both of the subunits of the vaccina capping complex, D1 and D12.

In some embodiments, the fusion protein of the disclosure comprises a mRNA capping enzyme protein comprising the amino acid sequence of the wild-type large subunit D1 (SEQ ID NO: 1) as shown in Table 1. In some embodiments, the amino acid sequence of the large subunit D1 has at least 70%, at least 75%, at least 80%, at least 85%, at least 90%, at least 95%, at least 96%, at least 97%, at least 98%, at least 99%, or 100% sequence identity to SEQ ID NO: 1.

In some embodiments, the D1 amino acid sequence is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 27.

In some embodiments, the fusion protein of the disclosure comprises a mRNA capping enzyme protein comprising the amino acid sequence of the wild-type small subunit D12 (SEQ ID NO: 2) as shown in Table 1. In some embodiments, the amino acid sequence of the small subunit D12 has at least 70%, at least 75%, at least 80%, at least 85%, at least 90%, at least 95%, at least 96%, at least 97%, at least 98%, at least 99%, or 100% sequence identity to SEQ ID NO: 2.

In some embodiments, the D12 amino acid sequence is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 28.

The capacity of a D1-D12 complex to cap a nascent mRNA with a Cap0 structure may be assessed by any method well known by the skilled person, such as a Dot Blot assay (as for example shown in Example 4).

### VP4 Capping Enzyme

As used herein the terms “VP4”, “VP4 capping enzyme”, or “bluetongue virus capping enzyme” can be used interchangeably and relate to the single-unit VP4 capping enzyme of Bluetongue virus (BTV; a dsRNA orbivirus of the family Reoviridae). VP4 is a 76 kDa protein and is encoded by BTV segment M4. This capping enzyme is likely able to homodimerize through a putative leucine zipper located near the carboxy terminus of the protein (Ramadevi et al. (1998), J Virol 72 (4): 2983-2990).

VP4 catalyzes all enzymatic steps required for mRNA m⁷GpppN capping synthesis. The stepwise process proceeds as follows: (1) hydrolysis of the 5′-triphosphate to a diphosphate by an RNA 5′-triphosphatase (RTPase); (2) addition of GMP via a 5′-5′ triphosphate linkage using a guanylyltransferase (GTase); and (3) transfer of a methyl group to the N7 position by a (guanine-N(7)-)-methyltransferase (N7MTase) to give cap 0. Subsequent methylation to form a cap 1 structure occurs on the 2′-hydroxyl of the ribose of the first nucleotide, catalyzed by a (nucleoside-2′-O—)-methyltransferase (2′OMTase). The methyltransferases utilize S-adenosyl-L-methionine (AdoMet) as the methyl donor, generating S-adenosyl-L-homocysteine (AdoHcy) (Sutton et al. (2007), Nat Struct Mol Biol. 14 (5): 449-451).

The fusion proteins described herein may comprise the full-length VP4 sequence or a fragment thereof, in particular an enzymatically active fragment thereof. In some embodiments, the fragment of the VP4 polypeptide has a length of at least 50 amino acids, at least 100 amino acids, at least 200 amino acids, at least 250 amino acids, at least 300 amino acids, at least 350 amino acids, at least 400 amino acids, at least 450 amino acids, at least 500 amino acids, at least 550 amino acids, or at least 600 amino acids. The fragment of the VP4 polypeptide retains at least the VP4 polypeptide enzymatic activity, in particular the capacity of catalyzing all enzymatic steps required for mRNA m⁷GpppN capping synthesis, i.e. to cap a nascent mRNA with a cap 1 structure. The capacity of an enzyme or fragment thereof to cap a nascent mRNA with a cap 1 structure may be assessed by any method well known by the skilled person, such as a Dot Blot assay.

In some embodiments, the VP4 polypeptide comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 16.

In some embodiments, the VP4 amino acid sequence is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 31.

mRNA Cap Specific 2′-O-Methyltransferase

As used herein, a mRNA cap specific 2′-O-methyltransferase (OMT) can convert a cap 0 structure into a cap 1 structure as shown in FIG. 1.

As described herein, VP39 is a mRNA cap specific 2′-O-methyltransferase. VP39 is derived from vaccinia virus and is about 39 kDa. At the 5′ mRNA end, VP39 acts as a cap-specific mRNA (nucleoside-2′-O—)-methyltransferase. In the initial steps of mRNA cap synthesis a cap 0 structure (m7G(5′)ppp(G/A)) is formed (Schnierle et al. (1992), PNAS, vol 89:2897-2901). VP39 acts upon the cap-0 structure by methylating the 2′-O position of the ribose of the first transcribed nucleotide in a S-adenosylmethionine (AdoMet)-dependent manner, converting cap-0 to the cap-1 (m7G(5′)ppp(Gm/Am)) form resulting in the cap-1 structure. (Schnierle, supra).

In some embodiments, the fusion protein of the disclosure comprises a VP39 enzyme protein comprising the amino acid sequence of the wild-type VP39 (SEQ ID NO: 6) as shown in Table 1. In some embodiments, the amino acid sequence of the VP39 has at least 70%, at least 75%, at least 80%, at least 85%, at least 90%, at least 95%, at least 96%, at least 97%, at least 98%, at least 99%, or 100% sequence identity to SEQ ID NO: 6.

In some embodiments, the VP39 amino acid sequence is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 30.

The fusion proteins described herein may comprise the full-length VP39 sequence or a fragment thereof, in particular an enzymatically active fragment thereof. In some embodiments, the fragment of the VP39 polypeptide has a length of at least 50 amino acids, at least 100 amino acids, at least 200 amino acids, at least 250 amino acids, or at least 300 amino acids. The fragment of the VP39 polypeptide retains at least the VP39 polypeptide enzymatic activity, in particular the cap-specific mRNA (nucleoside-2′-O—)-methyltransferase activity, enabling the conversion of a cap0 structure into a cap1 structure. The cap-specific mRNA (nucleoside-2′-O—)-methyltransferase activity of a compound may be assessed by any method well known by the skilled person, such as Dot Blot assay or MTase Glo Methyltransferase assay (as shown in Example 8).

In some embodiments, the fusion protein of the disclosure comprises a mutant VP39 enzyme protein. In some embodiments, the mutant VP39 enzyme protein comprises a C-terminal truncation of 26 amino acids (i.e., VP39-C26). The mutant VP39-C26 enzyme protein is thus a VP39 polypeptide fragment. In some embodiments, the mutant VP39 enzyme protein comprises the amino acid sequence of SEQ ID NO: 7 as shown in Table 1. In some embodiments, the amino acid sequence of the mutant VP39 has at least 70%, at least 75%, at least 80%, at least 85%, at least 90%, at least 95%, at least 96%, at least 97%, at least 98%, at least 99%, or 100% sequence identity to SEQ ID NO: 7.

### Tags

### Solubility Tags

In some embodiments, the fusion proteins of the disclosure comprising a mRNA capping enzyme (for instance, a D1, D12, D1-D12, VP39, and/or VP4 enzyme) comprise a solubility tag.

A used herein, a “solubility tag” refers to an amino acid sequence that is linked or fused to a protein of interest (e.g., mRNA capping enzyme) to improve protein solubility and expression. Examples of solubility tags can be found in Costa et al. (2014), Front. Microbiol., vol. 5 (63): 1-20 and include a small ubiquitin related modifier (SUMO) tag, Glutathione-S-transferase (GST) tag, maltose binding protein (MBP), as well as the Fh8 tag as described herein.

### SUMO

The solubility tag, small ubiquitin related modifier (SUMO), is a fusion tag which acts both as a chaperonin and as an initiator of protein folding. The SUMO tag is often used if the protein of interest traffics to inclusion bodies (Lee et al. (2008), Protein Sci, vol. 17 (7): 1241-1248). There are at least 4 SUMO paralogs in vertebrates, designated SUMO-1, SUMO-2, SUMO-3, and SUMO-4. SUMO-2 and SUMO-3 are structurally and functionally very similar and are distinct from SUMO-1. Previously, a SUMO solubility tagged D1-D12 for improved production in a E. coli Rosetta strain (Novagen) has been reported (U.S. Pat. No. 10,995,354 B2).

The SUMO amino acid sequence is SEQ ID NO: 9 as shown in Table 1.

### GST

As used herein, the GST tag is wild-type glutathione S-transferase (GST) or a variant thereof. GST tag can also be used as an affinity tag (for instance binding to glutathione-Sepharose beads). VP39 has been successfully expressed as a N-terminal tagged GST fusion protein (Schnierle et al. (1994), J Biol Chem., vol. 269 (30): 20700-20706). A GST tagged VP39 mutant with a C-terminal truncation of the last 26 amino acids (VP39-C26) has also been reported not to affect the 2′-O-Methyltransferase catalytic activity (Shi et al. (1996), RNA Journal, vol. 2:88-101).

The amino acid sequence for the GST tag is SEQ ID NO: 8 as shown in Table 1.

### MBP

As used herein, the MBP tag is wild-type maltose binding protein (MBP) or a variant thereof. MBP tag can also be used as an affinity tag (for instance binding to maltose-Sepharose beads).

The amino acid sequence for the MBP tag is SEQ ID NO: 15 as shown in Table 1.

### Fh8

As used herein, a Fh8 tag is any protein or a portion of a protein that can substitute for at least partial activity of a Fh8 tag. The Fh8 tag is an 8 kDa calcium-binding recombinant protein (GenBank ID AF213970) derived from the parasite Fasciola hepatica and has previously been used as part of the diagnosis procedure for parasitic infection with the same. Since the Fh8 is a calcium sensor protein that changes its structure upon calcium binding exposing its hydrophobic residues it can interact with target molecules such as phenyl-Sepharose hydrophobic resin (Costa et al. (2013), Protein Expression and Purification, vol. 92:163-170). The Fh8 tag has been used to enhance soluble protein expression (Costa et al. (2013), Appl Microbiol Biotechnol., vol. 97 (15): 6779-6791). The expressions “Fh8 polypeptide” and “Fh8 tag” as used herein are synonymous.

In some embodiments, the fusion protein of the disclosure comprises an Fh8 tag comprising the amino acid sequence of SEQ ID NO: 10 as shown in Table 1. In some embodiments, the amino acid sequence of the Fh8 tag has at least 70%, at least 75%, at least 80%, at least 85%, at least 90%, at least 95%, at least 96%, at least 97%, at least 98%, at least 99%, or 100% sequence identity to SEQ ID NO: 10. In some embodiments, the fragment of the Fh8 polypeptide has a length of at least 20 amino acids, at least 30 amino acids, at least 40 amino acids, at least 50 amino acids, or at least 60 amino acids. The fragment of the Fh8 polypeptide is a biologically active fragment of the Fh8 polypeptide. By “biologically active fragment of the Fh8 polypeptide”, it is herein meant that the Fh8 polypeptide retains at least some of the properties of the Fh8 polypeptide, in particular at least the Fh8 polypeptide solubilization activity. The solubilization activity of a compound may be assessed by any method well known by the skilled person, such as SDS-PAGE/western blot of total or insoluble fraction and soluble fractions using relevant primary antibody (e.g. raised against the solubilization tag or the fusion protein), a split GFP assay (kit commercialized at least by Sigma), or kinetic solubility assays (e.g. nephelometric assay, direct UV assay, or HPLC).

In some embodiments, the Fh8 tag amino acid sequence is encoded by a polynucleotide sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 29.

In some embodiments, the Fh8 polypeptide or fragment thereof is linked to the N-terminus or the C-terminus of the capping enzyme polypeptide (e.g., a D1, D12, D1-D12, VP39, and/or VP4 enzyme).

In some embodiments, the Fh8 polypeptide or fragment thereof is linked to the N-terminus of the capping enzyme polypeptide D1, D12, D1-D12, or VP39.

In some embodiments, the Fh8 polypeptide or fragment thereof is linked to the C-terminus of the capping enzyme polypeptide VP4.

In some embodiments, the Fh8 polypeptide or fragment thereof is linked to the N-terminus of the capping enzyme polypeptide D1, D12, D1-D12, or VP39 and to the C-terminus of the capping enzyme polypeptide VP4 enzyme.

### Periplasmic Tags

PhoA, lamb, malE, xynA, and pelB, as periplasmic tags, can also be used as solubility tags. They are signal peptides (also called signal sequences) that address the recombinant fusion protein in the periplasm of the host bacteria (Karyolaimos et al. (2019), Front. Microbial 10 (1511): 1-11; Karyolaimos and de Gier (2021), Front. Bioeng. Biotechnol 9:797334; Singh et al. (2013), Plos One 8 (5): e63442).

### Affinity Tags

In some embodiments, the fusion proteins of the disclosure comprising a mRNA capping enzyme (for instance, a D1, D12, D1-D12, VP39, and/or VP4 enzyme) further comprise an affinity tag.

An affinity tag is an amino acid sequence linked or fused to a protein of interest (e.g., mRNA capping enzyme) to facilitate purification of the protein of interest. Examples of solubility tags can be found in Costa et al. (2014), Front. Microbiol., vol. 5 (63): 1-20 and include His tag, MBP tag, or GST tag.

His tags are a consecutive series of six or more histidine residues (e.g., six to ten histidine residues). The most common his-tag is the hexahistidine tag, the Hiss tag, which has the molecular weight of 0.8 kDa. In some embodiments, the fusion proteins of the disclosure comprising a mRNA capping enzyme (for instance, a D1, D12, D1-D12, VP39, and/or VP4 enzyme) further comprise a His tag, for instance a His₆ tag.

The GST affinity tag is the same GST polypeptide as described above for the solubility tags. The MBP affinity tag is the same MBP polypeptide as described above for the solubility tags.

### Fusion Protein

In one aspect, disclosed herein is a fusion protein comprising a messenger RNA (mRNA) capping enzyme polypeptide linked to a Fh8 polypeptide or fragment thereof.

The Fh8 polypeptide is particularly as defined herein. The fragment of the Fh8 polypeptide is particularly as defined herein. Said fragment of the Fh8 polypeptide is a biologically active fragment as defined herein. In one embodiment, said fragment has a length of at least 20 amino acids, at least 30 amino acids, at least 40 amino acids, at least 50 amino acids, or at least 60 amino acids. The Fh8 polypeptide or the fragment thereof for example comprises an amino acid sequence comprising at least 90% identity to an amino acid sequence set forth in SEQ ID NO: 10.

The capping enzyme polypeptide may comprise (i) a vaccina virus D1 subunit and/or a vaccina virus D12 subunit, (ii) a vaccina virus VP39 polypeptide or fragment thereof, or (iii) a bluetongue virus VP4 polypeptide or fragment thereof. The vaccina virus D1 subunit, the vaccina virus D12 subunit, the vaccina virus VP39 polypeptide, the fragment of the vaccina virus VP39 polypeptide, the bluetongue virus VP4 polypeptide, and the fragment of bluetongue virus VP4 polypeptide are particularly as defined herein.

In the fusion protein as defined herein, the Fh8 polypeptide or fragment thereof may be linked to the N-terminus or the C-terminus of the capping enzyme polypeptide. In some embodiments, when the capping enzyme polypeptide comprises the VP4 polypeptide or a fragment thereof, the Fh8 polypeptide or fragment thereof is linked to the C-terminus of the capping enzyme polypeptide. In some embodiments, when the capping enzyme polypeptide comprises a vaccina virus D1 subunit and/or a vaccina virus D12 subunit, or comprises the VP39 polypeptide, the Fh8 polypeptide or fragment thereof is linked to the N-terminus of the capping enzyme polypeptide. In some embodiments, when the Fh8 polypeptide or fragment thereof is linked to the C-terminus of the capping enzyme polypeptide, the fusion protein comprises a linker between the capping enzyme polypeptide and the Fh8 polypeptide or fragment thereof.

In some embodiments, the fusion protein as defined herein comprises a capping enzyme polypeptide comprising a vaccina virus D1 subunit, optionally wherein the vaccina virus D1 subunit comprises an amino acid sequence comprising at least 90% identity to an amino acid sequence set forth in SEQ ID NO: 1; and/or the fusion protein comprises an amino acid sequence comprising at least 90% identity to an amino acid sequence set forth in SEQ ID NO: 3.

In some embodiments, the fusion protein as defined herein comprises a capping enzyme polypeptide comprising a vaccina virus D12 subunit, optionally wherein the vaccina virus D12 subunit comprises an amino acid sequence comprising at least 90% identity to an amino acid sequence set forth in SEQ ID NO: 2.

In some embodiments, the fusion protein as defined herein comprises a capping enzyme polypeptide comprising a vaccina virus D1 subunit as defined herein and a vaccina virus D12 subunit as defined herein, optionally wherein the vaccina virus D1 subunit comprises an amino acid sequence comprising at least 90% identity to an amino acid sequence set forth in SEQ ID NO: 1 and/or wherein the vaccina virus D12 subunit comprises an amino acid sequence comprising at least 90% identity to an amino acid sequence set forth in SEQ ID NO: 2.

In some embodiments, the fusion protein as defined herein comprises a capping enzyme polypeptide comprising a vaccina virus VP39 polypeptide or fragment thereof, optionally wherein the VP39 polypeptide comprises an amino acid sequence comprising at least 90% identity to an amino acid sequence set forth in SEQ ID NO: 6, and/or wherein the fusion protein comprises an amino acid sequence comprising at least 90% identity to an amino acid sequence set forth in SEQ ID NO: 4; or the VP39 polypeptide fragment comprises an amino acid sequence comprising at least 90% identity to an amino acid sequence set forth in SEQ ID NO: 7, and/or wherein the fusion protein comprises an amino acid sequence comprising at least 90% identity to an amino acid sequence set forth in SEQ ID NO: 5.

In some embodiments, the fusion protein as defined herein comprises a capping enzyme polypeptide comprising a bluetongue virus VP4 polypeptide or fragment thereof, optionally wherein the VP4 polypeptide comprises an amino acid sequence comprising at least 90% identity to an amino acid sequence set forth in SEQ ID NO: 16; and/or the fusion protein comprises an amino acid sequence comprising at least 90% identity to an amino acid sequence set forth in SEQ ID NO: 17, SEQ ID NO: 18, or SEQ ID NO: 22.

In some embodiments, the fusion protein comprises at least one additional tag, optionally selected from the group consisting of a solubility tag, a periplasmic tag, and an affinity tag. The solubility tag, the periplasmic tag, and the affinity tag may be as defined herein. The solubility tag is for example a SUMO tag, GST tag, or MBP tag. The affinity tag is for example a His tag, GST tag, or MBP tag. In one embodiment, the Fh8 polypeptide is the only solubility tag in the fusion protein. In one embodiment, the fusion protein does not comprise a SUMO tag, a GST tag, or a MBP tag.

In some embodiments, the fusion protein comprises at least one protease cleavage site, in particular to be able to remove tag(s) after protein purification. In one embodiment, the fusion protein comprises a protease cleavage site between the capping enzyme polypeptide and the Fh8 polypeptide or fragment thereof. Any protease cleavage site well-known by the skilled person may be used. The protease cleavage site is for example a TEV protease cleavage site.

In some embodiments, the fusion protein comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 3.

In some embodiments, the fusion protein comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 4.

In some embodiments, the fusion protein comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 5.

In some embodiments, the fusion protein comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 17.

In some embodiments, the fusion protein comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 18.

In some embodiments, the fusion protein comprises an amino acid sequence comprising at least 70%, at least 75%, at least 80%, at least 85%, at least 90%, at least 95%, at least 96%, at least 97%, at least 98%, at least 99%, or 100% sequence identity to an amino acid sequence set forth in SEQ ID NO: 22.

In some embodiments, the fusion protein is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 23.

In some embodiments, the fusion protein is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 24.

In some embodiments, the fusion protein is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 32.

In some embodiments, the fusion protein is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 33.

In some embodiments, the fusion protein is encoded by a polynucleotide sequence having at least 70%, at least 75%, at least 80%, at least 85%, at least 90%, at least 95%, at least 96%, at least 97%, at least 98%, at least 99%, or 100% sequence identity to SEQ ID NO: 37.

### Polynucleotide

In one aspect, disclosed herein is a polynucleotide comprising a nucleotide sequence that encodes a fusion protein as defined herein. Optionally, the nucleotide sequence is codon optimized.

In some embodiments, the polynucleotide comprises:
a) a nucleotide sequence encoding a fusion protein, wherein said fusion protein comprises a His tag, a Fh8 polypeptide, and a VP39 polypeptide and, optionally, having at least 90% identity to a nucleotide sequence set forth in SEQ ID NO: 23,
b) a nucleotide sequence encoding (i) a fusion protein, wherein said fusion protein comprises a His tag, a Fh8 polypeptide and a D1 subunit and (ii) a D12 subunit and, optionally, having at least 90% identity to a nucleotide sequence set forth SEQ ID NO: 24,
c) a nucleotide sequence encoding a fusion protein, wherein said fusion protein comprises a VP4 polypeptide, a Fh8 polypeptide and a His tag and, optionally, having at least 90% identity to a nucleotide sequence set forth SEQ ID NO: 32,
d) a nucleotide sequence encoding (i) a fusion protein, wherein said fusion protein comprises a VP4 polypeptide, a TEV protease cleavage site, a Fh8 polypeptide and a His tag and, optionally, having at least 90% identity to a nucleotide sequence set forth SEQ ID NO: 33,
e) a nucleotide sequence encoding (i) a fusion protein, wherein said fusion protein comprises a VP4 polypeptide, a Fh8 polypeptide and a His tag and, optionally, having at least 90% identity to a nucleotide sequence set forth SEQ ID NO: 36,
f) a nucleotide sequence encoding (i) a fusion protein, wherein said fusion protein comprises a His tag, a Fh8 polypeptide and a VP4 polypeptide and, optionally, having at least 90% identity to a nucleotide sequence set forth or SEQ ID NO: 37,
g) a nucleotide sequence encoding a fusion protein, wherein said fusion protein comprises a D1 subunit linked to a Fh8 polypeptide and, optionally, wherein the sequence encoding the D1 subunit has at least 90% identity to sequence SEQ ID NO: 27 and/or the sequence encoding the Fh8 polypeptide has at least 90% identity to sequence SEQ ID NO: 29,
h) a nucleotide sequence encoding a fusion protein, wherein said fusion protein comprises a D12 subunit linked to a Fh8 polypeptide and, optionally, wherein the sequence encoding the D12 subunit has at least 90% identity to sequence SEQ ID NO: 28 and/or the sequence encoding the Fh8 polypeptide has at least 90% identity to sequence SEQ ID NO: 29,
i) a nucleotide sequence encoding a fusion protein, wherein said fusion protein comprises a VP4 polypeptide linked to a Fh8 polypeptide and, optionally, wherein the sequence encoding the VP4 polypeptide has at least 90% identity to a sequence corresponding to nucleotides 1 to 1938 of SEQ ID NO: 31 and/or the sequence encoding the Fh8 polypeptide has at least 90% identity to sequence SEQ ID NO: 29,
j) a nucleotide sequence encoding a fusion protein, wherein said fusion protein comprises a VP39 polypeptide linked to a Fh8 polypeptide and, optionally, wherein the sequence encoding the VP39 polypeptide has at least 90% identity to sequence SEQ ID NO: 30 and/or the sequence encoding the Fh8 polypeptide has at least 90% identity to sequence SEQ ID NO: 29.
   In the polynucleotides disclosed herein, the His tag is optional. In the polynucleotides disclosed herein, the His tag is may be replaced with a different affinity tag.
   In the polynucleotides disclosed herein, the TEV protease cleavage site is optional. In the polynucleotides disclosed herein, the TEV protease cleavage site may be replaced with a different cleavage site.

### Vectors

In one aspect, disclosed herein are vectors comprising the polynucleotide sequences encoding the mRNA capping enzymes disclosed herein. The polynucleotides are, for example, as defined herein. The vectors include, but are not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

Expression of the polynucleotide sequences disclosed herein is driven by an RNA polymerase promoter. A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known. In some embodiments, the promoter is constitutive. In other embodiments, the promoter is inducible (e.g., an IPTG-inducible promoter).

Also disclosed herein are host cells (e.g., bacterial cells) comprising the vectors or RNA compositions disclosed herein.

Vectors can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Furthermore, the expression vectors preferably contain one or more selective marker genes to provide the phenotypic characteristics for selecting the transformed host cells, such as dihydrofolate reductase, neomycin resistance, or kanamycin resistance used for E. coli.

In some embodiments, the vector comprises:
a) a nucleotide sequence encoding a fusion protein as defined herein, wherein said fusion protein comprises (i) a vaccina virus D1 subunit and/or vaccina virus D12 subunit and (ii) a Fh8 polypeptide or fragment thereof,
b) optionally, a nucleotide sequence encoding (i) a fusion protein as defined herein comprising a vaccina virus D1 subunit linked to a Fh8 polypeptide or fragment thereof or (ii) a vaccina virus D1 subunit or fragment thereof, in particular if the fusion protein encoded by the nucleotide sequence a) does not comprise a vaccina virus D1 subunit,
c) optionally, a nucleotide sequence encoding (i) a fusion protein as defined herein comprising a vaccina virus D12 subunit linked to a Fh8 polypeptide or fragment thereof or (ii) a vaccina virus D12 subunit or fragment thereof, in particular if the fusion protein encoded by the nucleotide sequence a) does not comprise a vaccina virus D12 subunit, and
d) optionally, a nucleotide sequence encoding (i) a fusion protein as defined herein, wherein said fusion protein comprises a VP39 polypeptide or fragment thereof linked to a Fh8 polypeptide or fragment thereof or (ii) VP39 polypeptide or fragment thereof.

In some embodiments, the vector comprises a nucleotide sequence encoding a fusion protein as defined herein, wherein said fusion protein comprising a VP39 polypeptide or fragment thereof is linked to a Fh8 polypeptide or fragment thereof.

In some embodiments, the vector comprises a nucleotide sequence encoding a fusion protein as defined herein, wherein said fusion protein comprising a VP4 polypeptide or fragment thereof is linked to a Fh8 polypeptide or fragment thereof.

The vectors containing the suitable DNA sequences and suitable promoters or regulating sequences described above can be used for transforming suitable host cells to express proteins.

### IPTG Induction

The induction of the T7 promoter with isopropyl-β-D-1-thiogalactopyranoside (IPTG) as described herein is widely used for expression of large quantities of E. coli protein expression systems. Expression may be induced by the addition of IPTG or analogues of IPTG such as isobutyl-C-galactoside (IBCG), lactose or melibiose may also be suitable depending on the plasmid selected. The selection of inducer will depend on the expression system used and will be apparent to a person of ordinary skill in the art. Other inducers may be used and are described more fully elsewhere (e.g., Miller and Reznikoff (1978), The Operon, edition 448S). Inducers may be used individually or in combination.

E. coli Host Strains

Protein expression in Escherichia coli represents one of the most facile approaches for the preparation of non-glycosylated proteins for analytical and preparative purposes. Genome-scale engineering of E. coli have been employed to enhance recombinant protein expression to generate strains useful for protein expression. This engineering which primarily involves the introduction of DNA mutations that impact protein synthesis, degradation, secretion, or folding enable the generation of optimized E. coli expression strains in a manner analogous to metabolic engineering for the synthesis of low-molecular-weight compounds (Makino et al. (2011), Microb Cell Fact, vol 10:32). For example, the Artic Express strain (Agilent Technologies) improves protein processing at low temperatures. The BL21 (DE3) strain does not contain two proteases (Ion protease and OmpT) which reduce the degradation of heterologous proteins expressed in the cells. BL21 (DE3) is a strain widely used for production of recombinant proteins under the control of T7 RNA polymerase (Studier et al. (1986), J. Mol. Biol., vol 189:113-130).

Another example of a class of E. coli engineered host strains, are E. coli engineered strains that supply extra copies of rare tRNAs, such as the Rosetta strains (Invitrogen) and the BL21 Codon Plus strains (Novagen). A third example of a class of E. coli engineered host strains are mutant strains that facilitate disulfide bond formation and protein folding in the E. coli cytoplasm by render it oxidizing due to mutations in glutathione reductase (gor) and thioredoxin reductase (trxB) genes, and/or by co-production of Dsb proteins, such as the Origami strains (Novagen) or the Shuffle strain (New England Biolabs) (Lobstein et al. (2012), Microb Cell Fact., vol. 11:56). A fourth example of a class of E. coli host strains are those that improve the synthesis of membrane proteins such as the C41 and C43 (Avidis) BL21 (DE3) mutant strains (Dumon-Seignovert et al. (2004), Protein Expr Purif., vol 37 (1): 203-206).

Previously, soluble expression of His₆ tagged D1-D12 was reported to be low in E. coli BL21 (DE3) (Fuchs et al. (2016), RNA, vol. 22 (9): 1454-1466). A different group has reported a SUMO solubility tagged D1-D12 for improved production in a E. coli Rosetta strain (Novagen) (U.S. Pat. No. 10,995,354 B2).

Method of Capping an mRNA

In one aspect, disclosed herein is a method of capping an mRNA, comprising incubating the mRNA with a fusion protein as defined herein, wherein the capping enzyme polypeptide comprises a vaccinia virus D1 subunit and/or a vaccinia virus D12 subunit, in particular for obtaining a mRNA with a cap0 structure. The starting mRNA may be a nascent mRNA. If the fusion protein comprises a vaccinia virus D1 subunit but not a vaccinia virus D12 subunit, the mRNA is also incubated with (i) an additional fusion protein as defined herein, wherein the capping enzyme polypeptide comprises a vaccinia virus D12 subunit or (ii) a vaccinia virus D12 subunit. The fusion protein comprising the vaccinia virus D1 subunit is typically in the form of a complex with said additional fusion protein comprising a vaccinia virus D12 subunit or with said D12 subunit. Similarly, if the fusion protein comprises a vaccinia virus D12 subunit but not a vaccinia virus D1 subunit, the mRNA is also incubated with (i) an additional fusion protein as defined herein, wherein the capping enzyme polypeptide comprises a vaccinia virus D1 subunit or (ii) a vaccinia virus D1 subunit. The fusion protein comprising the vaccinia virus D12 subunit is typically in the form of a complex with said additional fusion protein comprising a vaccinia virus D1 subunit or with said D1 subunit. Said step of incubating the mRNA with a fusion protein, wherein the capping enzyme polypeptide comprises a vaccinia virus D1 subunit and/or a vaccinia virus D12 subunit, is carried out under conditions sufficient to cap the mRNA with a cap0 structure. Said conditions sufficient to cap the mRNA with a cap0 structure are well known by the skilled person and are those typically used when using a D1-D12 complex. For example, the mRNA is first denatured by heating at 65° C. for 5 minutes and then cooled down on ice for 5 minutes. The denatured mRNA is then incubated with the D1-D12 complex, for example at 37° C., for example for at least 30 minutes, in the presence of a buffer, GTP, S-adenosylmethionine (SAM) and, optionally, a ribonuclease inhibitor. For example, 0.1 pmol of complexed D1-D12 per 1 pmol of RNA substrate may be used.

In one aspect, disclosed herein is a method of capping an mRNA, comprising incubating the mRNA with a first fusion protein as defined herein, wherein the capping enzyme polypeptide comprises a vaccinia virus D1 subunit and/or a vaccinia virus D12 subunit and, after or simultaneously, with a second fusion protein as defined herein, wherein the capping enzyme polypeptide comprises a VP39 polypeptide or a fragment thereof, in particular for obtaining a mRNA with a cap1 structure. The starting mRNA may be a nascent mRNA. If the first fusion protein comprises a vaccinia virus D1 subunit but not a vaccinia virus D12, the mRNA is also incubated with (i) an additional fusion protein as defined herein, wherein the capping enzyme polypeptide comprises a vaccinia virus D12 subunit or (ii) a vaccinia virus D12 subunit. The fusion protein comprising the vaccinia virus D1 subunit is typically in the form of a complex with said additional fusion protein comprising a vaccinia virus D12 subunit or with said D12 subunit. Similarly, if the first fusion protein comprises a vaccinia virus D12 subunit but not a vaccinia virus D1 subunit, the mRNA is also incubated with (i) an additional fusion protein as defined herein, wherein the capping enzyme polypeptide comprises a vaccinia virus D1 subunit or (ii) a vaccinia virus D1 subunit. The fusion protein comprising the vaccinia virus D12 subunit is typically in the form of a complex with said additional fusion protein comprising a vaccinia virus D1 subunit or with said D1 subunit. Said step of incubating the mRNA with said first fusion protein and said second fusion protein is performed under conditions sufficient to cap the mRNA with a cap1 structure. Said conditions sufficient to cap the mRNA with a cap1 structure are well known by the skilled person and are those typically used when using D1-D12 complex and a VP39 polypeptide. For example, the mRNA is first denatured by heating at 65° C. for 5 minutes and then cooled down on ice for 5 minutes. The denatured mRNA is then incubated with the D1-D12 complex, for example at 37° C., for example for at least 30 minutes, in the presence of a buffer, GTP, S-adenosylmethionine (SAM) and, optionally, a ribonuclease inhibitor. For example, 0.1 pmol of complexed D1-D12 per 1 pmol of RNA substrate may be used. The second fusion protein, wherein the capping enzyme polypeptide comprises a VP39 polypeptide or a fragment thereof, is added either at the same time as the D1-D12 complex, or after, for example once a mRNA with a cap0 structure is obtained. When the second fusion protein is added after the D1-D12 complex, it may, for example, be incubated with the mRNA with a cap0 structure at 37° C., for example for at least one hour, in the presence of a buffer and S-adenosylmethionine (SAM). For example, 0.1 pmol of the second fusion protein per 1 pmol of mRNA substrate may be used. The mRNA with a cap0 structure may be first denatured, as disclosed above.

In one aspect, disclosed herein is a method of capping an mRNA, comprising incubating the mRNA with a fusion protein as defined herein, wherein the capping enzyme polypeptide comprises a bluetongue virus VP4 polypeptide or fragment thereof, in particular for obtaining a mRNA with a cap1 structure. The starting mRNA may be a nascent mRNA. Said step of incubating the mRNA with said fusion protein is performed under conditions sufficient to cap the mRNA with a cap1 structure. Said conditions sufficient to cap the mRNA with a cap1 structure are well known by the skilled person and are those typically used when using a bluetongue virus VP4 polypeptide. For example, the mRNA is first denatured by heating at 65° C. for 5 minutes and then cooled down on ice for 5 minutes. The denatured mRNA is then incubated with the fusion protein, for example at 37° C., for example for at least 1 hour, in the presence of a buffer, GTP, S-adenosylmethionine (SAM) and, optionally, a ribonuclease inhibitor. For example, 0.1 pmol of fusion protein per 1 pmol of mRNA may be used.

In one aspect, disclosed herein is a method of converting a cap0 structure on an mRNA to a cap1 structure, comprising incubating the mRNA with a fusion protein as defined herein, wherein the capping enzyme polypeptide comprises a VP39 polypeptide or a fragment thereof. The starting mRNA is a mRNA capped with a cap0 structure. Said step of incubating is performed under conditions sufficient to cap the mRNA with a cap1 structure. Said conditions sufficient to cap the mRNA are well known by the skilled person and are those typically used when using a VP39 polypeptide. For example, the mRNA with a cap0 structure is first denatured by heating at 65° C. for 5 minutes and then cooled down on ice for 5 minutes. The denatured mRNA is then incubated with the fusion protein, for example at 37° C., for example for at least one hour, in the presence of a buffer and S-adenosylmethionine (SAM). For example, 0.1 pmol of fusion protein per 1 pmol of mRNA may be used.

The step(s) of the above methods can be combined and/or performed combined with additional steps, as shown in the processes disclosed below.

In one aspect, disclosed herein is a process of preparing an mRNA comprising a step of capping, comprising:
a) incubating the mRNA with a fusion protein as defined herein, wherein the capping enzyme polypeptide comprises a vaccinia virus D1 subunit and/or a vaccinia virus D12 subunit, under conditions sufficient for the mRNA to be capped with a cap0 structure,
b) incubating the mRNA capped with a cap0 structure with a fusion protein as defined herein, wherein the capping enzyme polypeptide comprises a VP39 polypeptide or a fragment thereof, under conditions sufficient for the mRNA to be capped with a cap1 structure,
d) optionally purifying the capped mRNA,
e) optionally tailing the mRNA with a polyadenylation step, and
f) optionally purifying the capped polyadenylated mRNA.
Steps a) and b) may be performed as defined in the corresponding method provided herein. Step a) and step b) may be carried out simultaneously or step b) may be carried out after step a), in particular as defined herein.

In one aspect, disclosed herein is a process of preparing an mRNA comprising a step of capping, comprising:
a) incubating the mRNA with a fusion protein as defined herein, wherein the capping enzyme polypeptide comprises a bluetongue virus VP4 polypeptide or fragment thereof, under conditions sufficient for the mRNA to be capped with a cap1 structure,
b) optionally purifying the capped mRNA,
c) optionally tailing the mRNA with a polyadenylation step, and
d) optionally purifying the capped polyadenylated mRNA.
Step a) may be performed as defined in the corresponding method provided herein.

### RNA

The present capping enzyme compositions of the disclosure are capable of capping an RNA molecule (e.g., mRNA) that encodes a polypeptide of interest (e.g., an antigenic polypeptide). The RNA molecule may comprise at least one ribonucleic acid (RNA) comprising an ORF encoding a polypeptide of interest. In certain embodiments, the RNA is a messenger RNA (mRNA) comprising an ORF encoding a polypeptide of interest. In certain embodiments, the RNA (e.g., mRNA) further comprises at least one 5′ UTR, 3′ UTR, and/or a poly(A) tail.

### A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′ (A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′) A; G(5′)ppp(5′)G; m7G(5′)ppp(5′) A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies).

5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

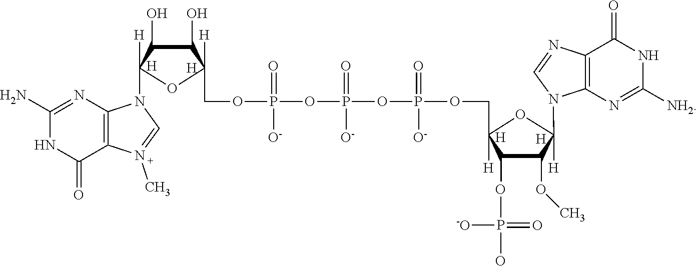

### B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 38) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 39 and reproduced below:

(SEQ ID NO: 39)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.

In some embodiments, the 3′UTR comprises a nucleic acid sequence set forth in SEQ ID NO: 40 and reproduced below:

CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

The 5′ UTR and 3′UTR are described in further detail in WO2012/075040, incorporated herein by reference.

### C. Polyadenylated Tail

As used herein, the terms “poly(A) sequence”, “poly(A) tail”, and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

(SEQ ID NO: 41)
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A) polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

### D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25 (2): 152-159; or in Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### Self-Replicating RNA and Trans-Replicating RNA

### Self-Replicating RNA:

Self-replicating RNA can be produced by using replication elements derived from, e.g., alphaviruses, and substituting the structural viral proteins with a nucleotide sequence encoding a protein of interest (e.g., an antigenic polypeptide). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) a protein of interest. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the protein of interest, such that the subgenomic transcript encodes the protein of interest rather than the structural alphavirus virion proteins. Self-replicating RNA are described in further detail in WO2011005799, incorporated herein by reference.

### Trans-Replicating RNA:

Trans-replicating RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an antigenic prokaryotic polypeptide). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### EMBODIMENTS OF THE DISCLOSURE

Embodiment 1. A fusion protein comprising a messenger RNA (mRNA) capping enzyme polypeptide linked to a Fh8 polypeptide or fragment thereof.

Embodiment 2. The fusion protein of Embodiment 1, wherein the fragment of the Fh8 polypeptide retains solubilization activity of the Fh8 polypeptide.

Embodiment 3. The fusion protein of Embodiment 1 or 2, wherein the fragment of the Fh8 polypeptide is at least 20 amino acids, at least 30 amino acids, at least 40 amino acids, at least 50 amino acids, or at least 60 amino acids, in length.

Embodiment 4. The fusion protein of any one of Embodiments 1 to 3, wherein the Fh8 polypeptide or fragment thereof comprises an amino acid sequence comprising at least 90% identity to an amino acid sequence set forth in SEQ ID NO: 10.

Embodiment 5. The fusion protein of any one of Embodiments to 4, wherein the Fh8 polypeptide or fragment thereof is linked to the N-terminus or the C-terminus of the capping enzyme polypeptide.

Embodiment 6. The fusion protein of any one of Embodiments 1 to 5, wherein the capping enzyme polypeptide comprises a vaccina virus D1 subunit.

Embodiment 7. The fusion protein of Embodiment 6, wherein the vaccina virus D1 subunit comprises an amino acid sequence comprising at least 90% identity to an amino acid sequence set forth in SEQ ID NO: 1.

Embodiment 8. The fusion protein of Embodiment 6 or 7, wherein the fusion protein comprises an amino acid sequence comprising at least 90% identity to an amino acid sequence set forth in SEQ ID NO: 3.

Embodiment 9. The fusion protein of any one of Embodiments 1 to 8, wherein the capping enzyme polypeptide comprises a vaccina virus D12 subunit.

Embodiment 10. The fusion protein of Embodiment 9, wherein the vaccina virus D12 subunit comprises an amino acid sequence comprising at least 90% identity to an amino acid sequence set forth in SEQ ID NO: 2.

Embodiment 11. The fusion protein of any one of Embodiments 1 to 5, wherein the capping enzyme polypeptide comprises a vaccina virus VP39 polypeptide or fragment thereof.

Embodiment 12. The fusion protein of Embodiment 11, wherein the fragment of the capping enzyme polypeptide is enzymatically active.

Embodiment 13. The fusion protein of Embodiment 11 or 12, wherein the fragment of the capping enzyme polypeptide is at least 50 amino acids, at least 100 amino acids, at least 200 amino acids, at least 250 amino acids, or at least 300 amino acids, in length.

Embodiment 14. The fusion protein of any one of Embodiments 11 to 13, wherein the VP39 polypeptide comprises an amino acid sequence comprising at least 90% identity to an amino acid sequence set forth in SEQ ID NO: 6, or wherein the fusion protein comprises an amino acid sequence comprising at least 90% identity to an amino acid sequence set forth in SEQ ID NO: 4.

Embodiment 15. The fusion protein of Embodiment 14, wherein the VP39 polypeptide fragment comprises an amino acid sequence comprising at least 90% identity to an amino acid sequence set forth in SEQ ID NO: 7, or wherein the fusion protein comprises an amino acid sequence comprising at least 90% identity to an amino acid sequence set forth in SEQ ID NO: 5.

Embodiment 16. The fusion protein of any one of Embodiments 1 to 5, wherein the capping enzyme polypeptide comprises a bluetongue virus VP4 polypeptide or fragment thereof.

Embodiment 17. The fusion protein of Embodiment 16, wherein the fragment of the capping enzyme polypeptide is enzymatically active.

Embodiment 18. The fusion protein of Embodiment 17, wherein the fragment of the capping enzyme polypeptide has RNA triphosphatase enzymatic activity, guanylyltransferase enzymatic activity, or methyltransferase activity, or any combination thereof.

Embodiment 19. The fusion protein of any one of Embodiments 16 to 18, wherein the fragment of the capping enzyme polypeptide is at least 50 amino acids, at least 100 amino acids, at least 200 amino acids, at least 250 amino acids, at least 300 amino acids, at least 350 amino acids, at least 400 amino acids, at least 450 amino acids, at least 500 amino acids, at least 550 amino acids, or at least 600 amino acids, in length.

Embodiment 20. The fusion protein of any one of Embodiments 16 to 19, wherein the VP4 polypeptide comprises an amino acid sequence comprising at least 90% identity to an amino acid sequence set forth in SEQ ID NO: 16.

Embodiment 21. The fusion protein of Embodiment 16 or 20, wherein the fusion protein comprises an amino acid sequence comprising at least 90% identity to an amino acid sequence set forth in SEQ ID NO: 17, SEQ ID NO: 18, or SEQ ID NO: 22.

Embodiment 22. A polynucleotide comprising a nucleotide sequence that encodes the fusion protein according to any one of Embodiments 1 to 21.

Embodiment 23. The polynucleotide of Embodiment 22, wherein the nucleotide sequence is codon optimized.

Embodiment 24. The polynucleotide of Embodiment 22 or 23, wherein the nucleotide sequence comprises at least 90% identity to a nucleotide sequence set forth in SEQ ID NO: 23, SEQ ID NO: 24, SEQ ID NO: 32, SEQ ID NO: 33, or SEQ ID NO: 37.

Embodiment 25. An expression vector comprising the polynucleotide of any one of Embodiments 22 to 24.

Embodiment 26. A host cell comprising the expression vector of Embodiment 25.

Embodiment 27. The host cell of Embodiment 26, wherein the host cell is an E. coli cell.

Embodiment 28. The host cell of Embodiment 27, wherein the E. coli cell is a BL21 (DE3) or Origami E. coli cell strain.

Embodiment 29. A method of expressing a fusion protein, comprising culturing the host cell of any one of Embodiments 26-28 under conditions sufficient to express the fusion protein.

Embodiment 30. The method of Embodiment 29, wherein the fusion protein is further isolated from the host cell.

Embodiment 31. A method of capping an mRNA, comprising incubating the mRNA with the fusion protein of any one of Embodiments 1-10 under conditions sufficient to cap the mRNA with a cap0 structure.

Embodiment 32. A method of converting a cap0 structure on an mRNA to a cap1 structure, comprising incubating the mRNA with the fusion protein of any one of Embodiments 1-5 or 11-15 under conditions sufficient to cap the mRNA.

Embodiment 33. A method of capping an mRNA, comprising incubating the mRNA with the fusion protein of any one of Embodiments 1-5 or 16-21 under conditions sufficient to cap the mRNA with a cap1 structure.

Embodiment 34. A method of capping an mRNA, comprising incubating the mRNA with the fusion protein of any one of Embodiments 6-10 and with the fusion protein of any one of Embodiments 11-15 under conditions sufficient to cap the mRNA with a cap1 structure.

Embodiment 35. The method of Embodiment 34, wherein the fusion protein of any one of Embodiments 6-10 is incubated prior to or simultaneously with the fusion protein of any one of Embodiments 11-15.

Embodiment 36. A process of preparing an mRNA comprising a step of capping, comprising: a) incubating the mRNA with the fusion protein of any one of Embodiments 1-10 under conditions sufficient for the mRNA to be capped with a cap0 structure, b) incubating the mRNA capped with a cap0 structure with the fusion protein of any one of Embodiments 11-15 under conditions sufficient for the mRNA to be capped with a cap1 structure, d) optionally purifying the capped mRNA, e) optionally tailing the mRNA with a polyadenylation step, and f) optionally purifying the capped polyadenylated mRNA.

Embodiment 37. A process of preparing an mRNA comprising a step of capping, comprising: a) incubating the mRNA with the fusion protein of any one of Embodiments 1-5 or 16-21 under conditions sufficient for the mRNA to be capped with a cap1 structure, b) optionally purifying the capped mRNA, c) optionally tailing the mRNA with a polyadenylation step, and d) optionally purifying the capped polyadenylated mRNA.

Embodiment 38. A capped MRNA obtained by the method of any one of Embodiments 31-35, or by the process of Embodiment 36 or 37.

In order that this disclosure may be better understood, the following examples are set forth. These examples are for purposes of illustration only and are not to be construed as limiting the scope of the disclosure in any manner.

### EXAMPLES

### Example 1: Design of Expression Plasmids for Increasing the Solubility of Vaccinia Capping Enzyme D1/D12

### Background

E. coli fusion tags can improve protein production titers, solubility and folding, and ultimately facilitate protein purification. A fusion tag that is designed to improve protein solubility is also called a solubility tag. Notwithstanding, fusion tags/solubility tags need to be tailored to the protein of interest because each tag may target a different step in the protein purification procedure but also because the protein of interest will have unique attributes which can create hurdles for purification.

### Plasmid Design for D1/D12

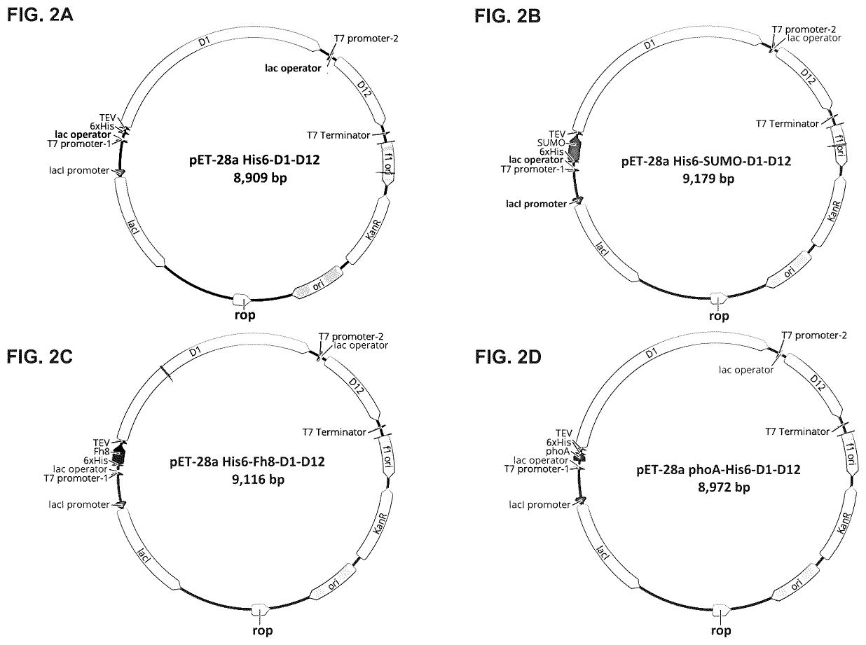

To analyze whether the type of solubility tag impacts the solubility of the vaccinia capping complex, D1-D12, the following four plasmids were designed. The plasmid maps are shown in FIG. 2A-FIG. 2D.

To ensure stability of the D1-D12 complex, a dual T7 promoter system was used to drive expression of the two subunits of vaccinia capping enzyme, D1 and D12 in a pET28 vector which is suitable for transformation in E. coli. Untagged D12 is co-purified in the D1-D12 complex formed upon its expression (Fuchs et al. (2016), RNA, vol. 22 (9): 1454-1466). A N′ terminal His₆ tag was added to D1 (FIG. 2A, control, called pET-28a His6-D1-D12), a N′ terminal SUMO fusion tag was added to D1 (FIG. 2B, called pET-28a His6-SUMO-D1-D12), a N′ terminal Fh8 fusion tag was added to D1 (FIG. 2C, called pET-28a His6-Fh8-D1-D12), or a N′ terminal phoA followed by a Hiss tag was added to D1 (FIG. 2D, called pET-28a phoA-His6-D1-D12). The PhoA periplasmic tag was expressed at the N-terminus and is cleaved in the periplasm of the bacteria thus exposing the His tag.

Other fusion periplasmic tags which have the same mechanism of cleavage in the bacterial periplasm as PhoA, including, lamb, malE, xynA, and pelB were similarly designed. All the D1-D12 nucleotide sequences were codon optimized (by codon optimization method A), except for the SUMO tag, where a codon optimized D1-D12 sequence construct (His₆-SUMO-D1-D12.1) and a non-codon optimized D1-D12 sequence construct (His₆-SUMO-D1-D12) were made.

### Methods

Transformation and generation of glycerol stocks. The plasmids were then used to transform competent cells following the manufacturer's instructions. Briefly, competent E. coli cells stocks (ArcticExpress (DE3), BL21 (DE3), Origami, Shuffle), stored in a −80° C. freezer, were thawed on ice and transferred to BD Falcon round bottom tube on ice. To increase transformation efficiency, β-mercaptoethanol was diluted 1:10 with dH2O and 2 μl was added to competent E. coli cells prior to the transformation procedure. Subsequently, cells were incubated on ice for 10 min. Next, 5 ng plasmid (1 μl of 5 ng/μl stock) were added to cells and incubated on ice 30 min. Cells were then heat pulsed in a 42° C. water bath for 20 seconds and transferred to ice for 2 min. Preheated 0.9 ml (37° C.) LB medium was added to the cell plus plasmid mix and the tube was incubated at 37° C. for 1 hour, shaking at 220 rpm. 200 μl of each transformation reaction as spread on LB plates and incubated at 37° C. overnight. The next day, 3 colonies were picked from each transformation and inoculated into 1 ml of LB media and grown in deep well 96-well plate. Samples were incubated overnight shaking at 250 rpm at 37° C. All the media are supplemented with the appropriate antibiotics.

To generate the glycerol stock plate, 1:50 dilution of the overnight culture from the deep well 96-well plate was taken. 100 μl sterile 80% glycerol and 100 μl culture are added to wells of a clear 96 well cell culture plate. Plates are prepared and sealed with Thermo Fisher Adhesive Sealing Sheets and stored at −80° C.

### Example 2: Soluble Expression of N-Terminal Solubility Tagged D1/D12

### Background

The D1-D12 expression plasmids designed in Example 1 were used to transform several E. coli host strains to test whether host cell selection could improve soluble protein expression. The E. coli-engineered host strains included, Artic Express, BL21 (DE3), Shuffle, and Origami.

### Methods

Bacterial culture and induction conditions. To prepare the growth culture for enzyme expression, each clone was expanded so that each transformation had 3 clones with 3 technical replicates. Back dilute bacterial culture 1:50 was grown for 3 hours at 37° C. with 220 rpm shaking. Upon induction with 50 UM-1 mM IPTG, the temperature was lowered to 12-22° C. After overnight induction, cells were harvested by centrifugation of plates at 3500 rpm for 10 min. Supernatant was decanted and cell pellets were stored at −80° C.

Evaluation of enzyme expression. 0.5-1 ml of cell sample was spun down at 4000 g for 10 min. Cell pellets were lysed using BugBuster Master Mix supplemented with protease inhibitors for 20 min at room temperature. The whole cell lysate sample was then spun down and pelleted for 10 min at 16000 g. Protein concentration was assayed using a BCA, samples were diluted with 0.1×JESS sample buffer to approximately 100 μg/ml. JESS standard pack reagents were prepared as described by the manufacturer's instructions. Samples were mixed with 5× fluorescent master mix and denatured at 95° C. for 5 min. Samples were loaded onto an assay plate as suggested by the manufacturer using a 1/10 dilution of a primary anti-his tag antibody and 1/20 of a secondary fluorescently labelled antibody. Assay plates were spun for 5 min at 2500 g and loaded into JESS instrument.

### Results

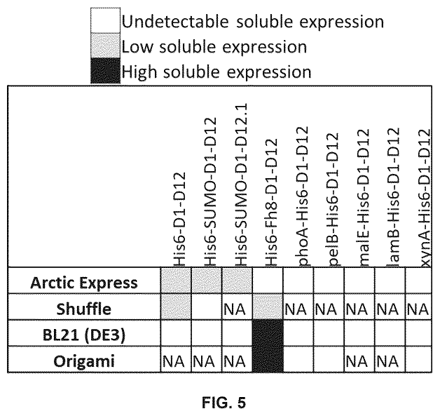

All the constructs were first screened for expression in the whole cell. All the constructs and strains showed a high whole cell expression (data not shown) and were further evaluated for soluble expression. The soluble expression screening results are summarized in FIG. 5.

Soluble expression of His₆-D1-D12 was low in Arctic Express and Shuffle strains and undetectable in BL21 (DE3). SUMO-tagged D1 and the codon optimized variant, His₆-SUMO-D1-D12.1, had a similar soluble expression pattern in the E. coli Artic Express or BL21 (DE3) strains.

Advantageously, the soluble expression pattern of Fh8-tagged D1-D12 was significantly higher than the His₆-D1-D12 construct which did not have a solubility tag or the SUMO-tagged construct in E. coli BL21 (DE3) and E. coli Origami. None of the other tested solubility tags, i.e., the periplasmic phoA, pelB, malE, lamb or XynA tags, improved the soluble expression of the fused protein D1-D12. See FIG. 5, column 4.

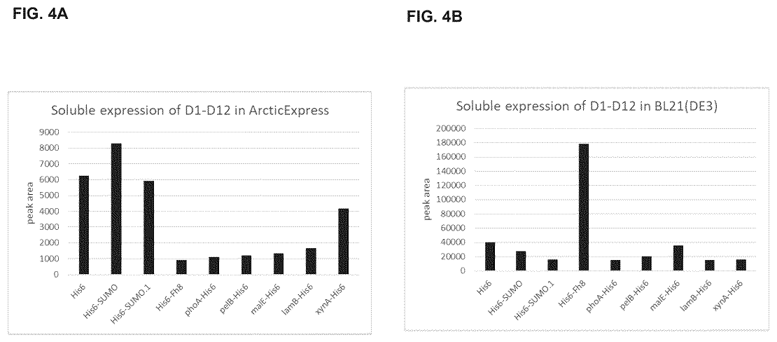

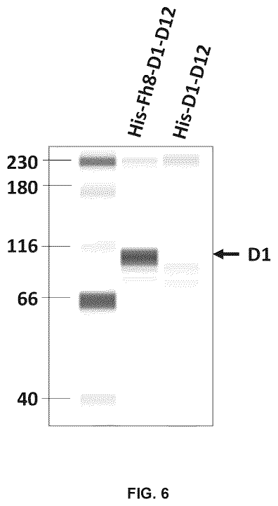

E. coli strain selection also impacted the soluble expression pattern of the Fh8 tagged constructs as shown in FIG. 4A and FIG. 4B. As shown in FIG. 4B the Fh8 tagged construct outperformed the other constructs when expressed in E. coli BL21 (DE3). The extent of Fh8-tagged-D1-D12 construct improvement over the His₆-D1-D12 construct which was not fused to a solubility tag is also shown in the JESS gel image of FIG. 6.

### Example 3: Optimization of Induction Conditions for His₆-Fh8-D1-D12 Soluble Expression

### Background

Besides expression host strain, cultivation conditions (i.e., temperature, pH, induction time, and inducer concentration) may have a significant impact on the production of soluble protein. Induction at low temperature as well as induction with reduced amount of inducing agent, IPTG, can improve the soluble protein yield. The inventors tested another critical parameter, i.e., the phase of the growth of the culture at induction, which correlates with cell density and can be measured at OD 600 nm for most E. coli strains.

### Methods

To prepare the growth culture for enzyme expression, overnight bacterial culture was diluted 1/250 in fresh LB media and grown at 37° C., shaking at 220 rpm until the culture reached OD values of 0.1-0.4 read at OD₆₀₀. Then the temperature was lowered to 16° C. and induced with 0.05-0.1 mM IPTG. After overnight induction, cells were harvested by centrifuging plates at 6000 g for 20 min, decant supernatant, and store cell pellets at −80° C.

### Results

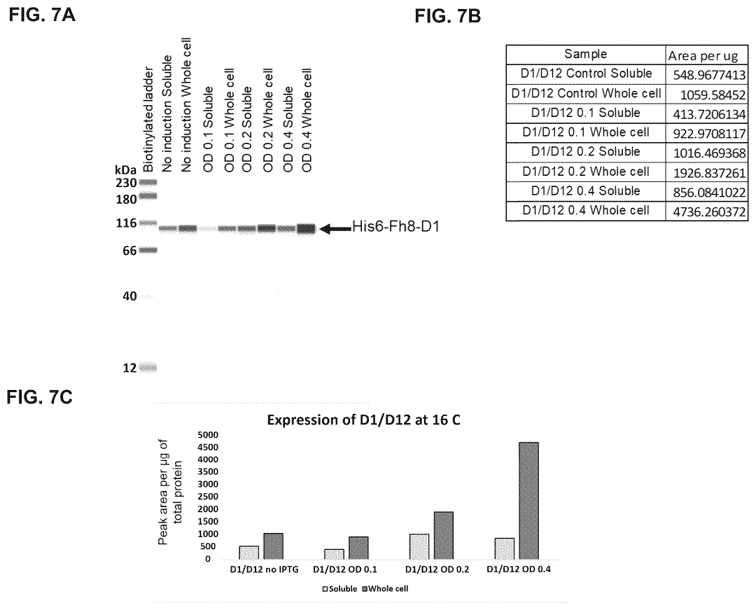

To optimize the induction conditions, the E. coli BL21 (DE3)/pET28aHis₆-Fh8-D1-D12 cells were cooled down to 16° C. and then induced with IPTG at different cell densities (0.1-0.4) at OD₆₀₀ or left uninduced. The results are summarized in FIG. 7. The uninduced sample demonstrated some low expression due to unspecific promoter activity. Notwithstanding, the fraction of soluble protein reached up to 50% as shown in FIG. 7B and FIG. 7C. The best expression level and soluble fraction was observed with IPTG induction at 0.2 OD₆₀₀ with the solubility yield being twice as much as the uninduced control. Induction at 0.4 OD₆₀₀ resulted in predominantly an insoluble product.

### Example 4: Recombinant His₆-Fh8-D1-D12 Enzyme Activity is Improved Over Commercially Available D1-D12

### Background

Any modification of a protein can be detrimental to its biological activity, this is especially true for enzymes, where the catalytic site can become inefficient. The enzymatic activity of recombinant His₆-Fh8-D1-D12 enzyme produced in Example 2 was compared to commercially available D1-D12 enzyme (NEB) to evaluate whether the recombinant His₆-Fh8-D1-D12 enzyme could be an attractive alternative to commercially available mRNA capping enzymes.

### Methods

Capping Reaction. 12.6 μl of the RNA substrate was mixed with 17.4 μl of DEPC-water, incubated for 5 minutes at 65° C. and then cooled down for 5 minutes on ice. Capping enzymes were diluted (1:10, 1:20, or 1:100) in capping buffer supplemented with 0.1 mg/ml BSA. The capping buffer was 50 mM Tris-HCl PH 8.0, 5 mM KCl, 1 mM MgCl2 and 1 mM DTT. To start the reaction, 5 μl of the RNA substrate was added to the samples containing either the NEB vaccinia capping enzyme or the His₆-Fh8-D1-D12 enzyme at various concentrations. The reaction was incubated at 0, 10, 20, and 30 minutes in a 37° C. water bath. The reaction was supplemented with 0.5 mM GTP, 0.2 mM S-adenosylmethionine (SAM), 0.1 pmol of D1/D12 per 1 pmol of RNA substrate was used. To stop the reaction, 140 μl of extraction buffer was added to 10 μl of reaction sample. Subsequently, 150 μl of phenol-chloroform mixture was added, mixed, and vortexed. Samples were then spun down at 12000 g for 10 minutes to separate phases. The aqueous (upper) phase was added to 1 μl of glycogen and 600 μl of cold ethanol. Samples were then incubated overnight at −20° C.

Dot Blot Analysis. RNA capped samples prepared from the capping reaction detailed above were spun down to remove the ethanol and subsequently dissolved in 2 μl of DEPC-water. The nitrocellulose membrane was wet in PBS for 5 minutes and left to air dry. RNA capped concentration standard was also prepared in DEPC-water. 1 μl of samples and standards were added to membrane. Subsequently, spotted RNA was crosslinked to membrane for 2 minutes using a UVP cross-linker. The crosslinked RNA-membrane was then washed to release unbound RNA for 15 minutes in PBS-T buffer on an orbital shaker and blocked in blocking solution for 1 hour at room temperature on an orbital shaker. Primary antibody, anti-7 mG cap mouse monoclonal antibody (MBL), was diluted in 1:1000 in blocking solution, added, and incubated overnight at 4° C. The next day the membrane was washed for 5 minutes in PBS-T three times. 1.5 ml of the secondary antibody, anti-mouse HRP conjugated antibody (ThermoFisher), diluted in 1:5000 of blocking solution was incubated for 1 hour at room temperature. Membranes were washed 15 minutes in PBS-T on the orbital shaker, three times. Capped RNA was visualized with the ECL prime kit according to manufacturer's instructions and an iBright gel imager was used to take an image of the membrane.

### Results

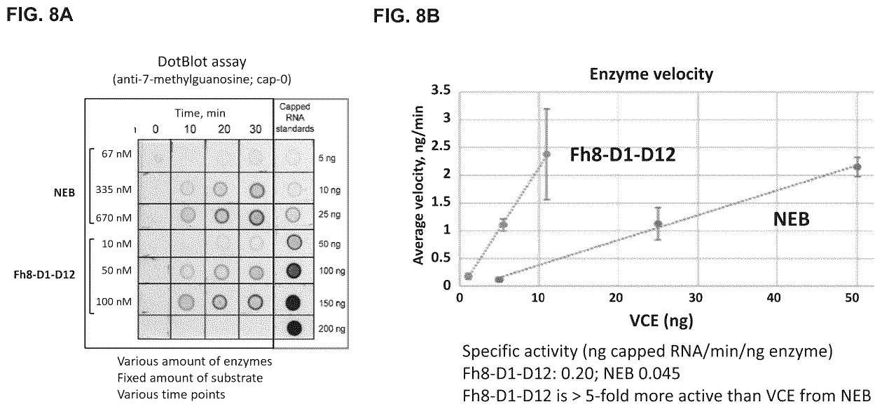

Unexpectedly, as quantified by the dot blot shown in FIG. 8A the enzymatic activity of the recombinant His₆-Fh8-D1-D12 enzyme was improved over the commercially available RNA capping enzyme. Further, the recombinant His₆-Fh8-D1-D12 enzyme reached a similar reaction velocity at five times lower the concentration required from the commercially available mRNA capping enzyme (FIG. 8B). As such, the recombinant His₆-Fh8-D1-D12 enzyme has improved enzymatic activity when compared to commercially available counterpart.

### Example 5: Design of Expression Plasmids for Increasing the Solubility of Vaccinia Capping Enzyme VP39

### Background

VP39 has been successfully expressed as a N-terminal tagged GST fusion protein (Schnierle et al. (1994), J Biol Chem., vol. 269 (30): 20700-20706). A GST tagged VP39 mutant with a C-terminal truncation of the last 26 amino acids (VP39-C26) has also been reported not to affect the 2′-O-Methyltransferase catalytic activity (Shi et al. (1996), RNA Journal, vol. 2:88-101).

To see whether addition of Fh8 solubility tag could also improve the purification of VP39, the Fh8 fusion tag was compared to GST tag using full-length VP39 as well as the VP39-C26 mutant.

### Plasmid Design for VP39

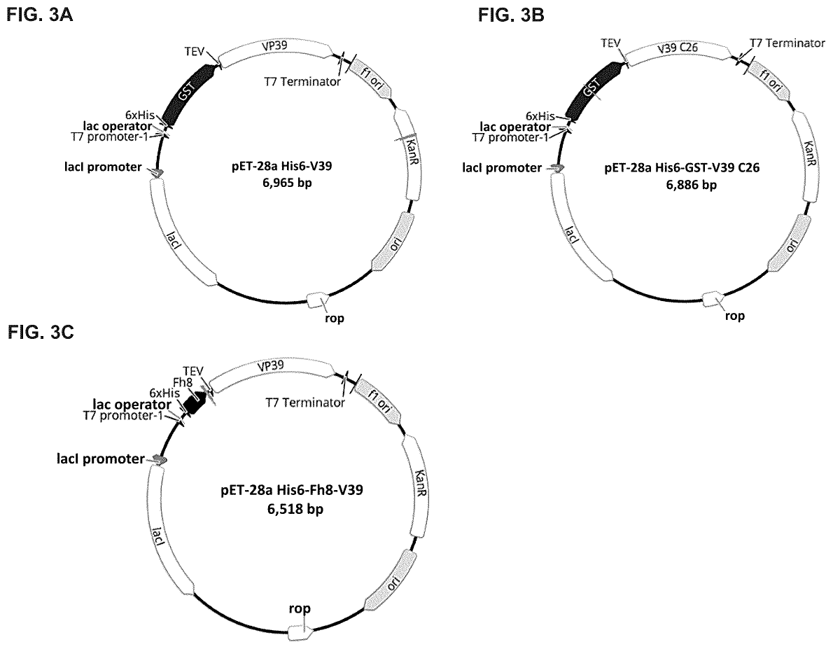

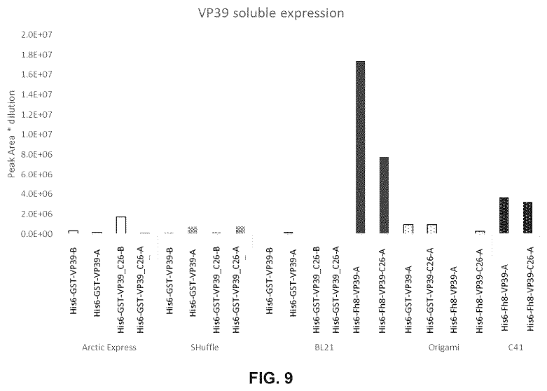

Like in Example 1, a pET-28a expression plasmid was selected. The genetic elements included a T7 promoter and an adjacent lac operator sequence to suppress uninduced expression. The following three plasmid maps containing VP39 or VP39-C26 were designed as shown in FIG. 3A-FIG. 3C. A N-terminal Hiss tag was added to VP39 (FIG. 3A, control, called pET-38a His6-V39). A N-terminal His6 tag and a GST solubility tag was added to VP39-C26 (FIG. 3B, called pET-28a His6-GST-V39 C26). A N-terminal His6 tag and a Fh8 solubility tag was added to VP39 (FIG. 3C, called pET-28a His6-Fh8-V39). Other plasmids were designed with the constructs His6-GST-VP39 and His6-Fh8-VP39-C26. All constructs were codon optimized either by method A or by method B indicated on each X-axis construct label by a terminal notation of “A” or “B” on FIG. 9.

Transformation conditions and glycerol stock preparation were the same as disclosed in Example 1.

### Example 6: Soluble Expression of VP39

VP39 expression plasmids as described in Example 5 were used to transform several E. coli host strains. Methods and conditions were the same as described for the soluble expression of D1-D12 constructs in Example 2.

### Results

The soluble expression pattern in several E. coli host strains for the His₆-GST-VP39, His₆-GST-VP39-C26, the His₆-Fh8-VP39, and the His₆-Fh8-VP39-C26-constructs, with capping enzyme sequences codon optimized according to method A or method B are shown in FIG. 9. Surprisingly, in E. coli BL21 (DE3), the Fh8-tagged VP39 and Fh8-tagged VP39-C26 mutant produced more than 10-fold higher soluble enzymes than constructs which did not contain a fusion tag or which contain the GST fusion tag, highlighting the advantage of using the Fh8 solubility tag for VP39 protein purification.

### Example 7: VP39-Fh8 Production in a Fermenter

### Background

The His₆-Fh8-VP39-C26-A construct was grown in a BioFlo fermentation system. Induction parameters were 0.1 mM IPTG at 22° C.

### Results

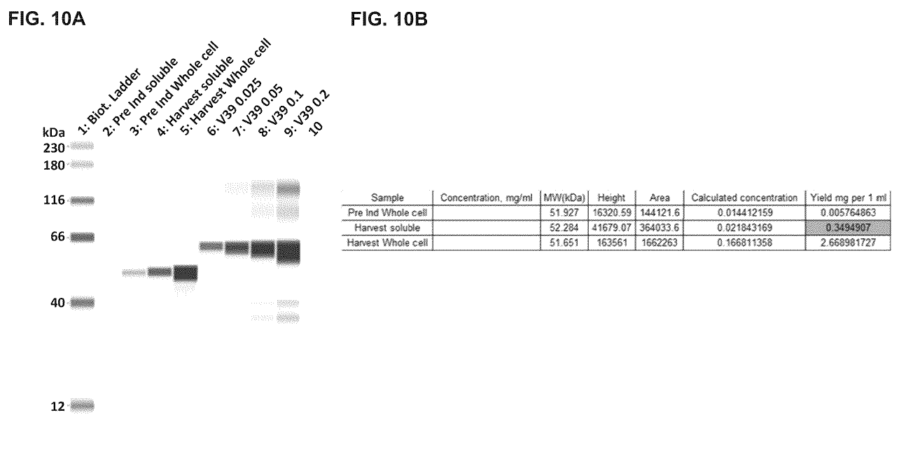

Samples before and after induction with IPTG were collected and analyzed for the yield of soluble protein using quantitative JESS gel as shown in FIG. 10A. Using purified GST-tagged VP39 as a standard, the amount of soluble VP39 enzyme was estimated as shown in FIG. 10B. The yield of soluble protein was 0.35 mg per ml of culture or about 1.4 g total for this fermentation run. This Example demonstrates that the Fh8-tagged VP39 is scalable for future industrial fermentation procedures.

### Example 8: Recombinant Fh8-VP39-C26 Activity is Improved Over Commercially Available VP39

### Background

The enzymatic activity of recombinant Fh8-VP39-C26 enzyme expressed in Example 6 and 7 was compared to commercially available VP39 enzyme (NEB) to evaluate whether the recombinant Fh8-VP39-C26 enzyme could be an attractive alternative to commercially available mRNA cap 2′-O-methyltransferases.

### Methods

Methyltransferase Activity Assay. Cap 0 substrate RNA was prepared as described in Example 4. Subsequently, Cap 0 substrate RNA was incubated with either the Fh8-VP39-C26 enzyme or the commercially available VP39 enzyme in the experimental set up described in the MTase-Glo Methyltransferase Assay (Promega) manufacturer's instructions. Briefly, after the methyltransferase reaction is complete, the MTas-Glo reagent is added to convert the reaction product, S-adenosyl homocysteine (SAH) to ADP. The MTase Glo detection solution is then added to convert ADP to ATP which is detected via a luciferase reaction. Luminescence was read using a Cytation plate reader in luminescence mode. Incubation with VP39 may be performed subsequent to Cap0 generation with D1-D12, or simultaneously with D1-D12.

Purify mRNA. Capped (Cap-1) RNA was purified by Monarch® RNA Cleanup Kit (50 μg) from NEB (Cat #T2040S) according to manufacturer's instructions.

### Results

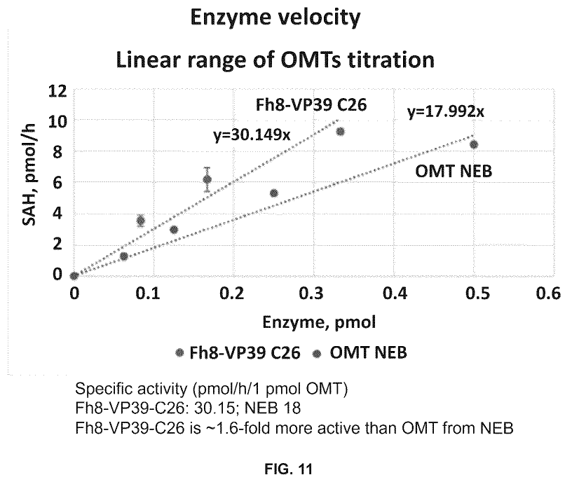

The methyltransferase activity of the recombinant Fh8-VP39-C26 enzyme or the commercially available VP39 enzyme (NEB) was tested using the MTase-Glo Methyltransferase assay (Promega). Assay results were displayed in terms of the reaction velocity. The initial reaction velocity of the recombinant Fh8-VP39-C26 enzyme is 30.15 pmol/h per 1 pmol of enzyme. A similar concentration of the commercially available VP39 enzyme produced 1.66 less amount of SAH indicative of a lower enzyme activity. Accordingly, the recombinant Fh8-VP39-C26 enzyme has improved methyltransferase activity when compared to a commercially available counterpart (FIG. 11).

### Example 9: Design and Soluble Expression of Solubility Tagged Bluetongue Virus Capping Enzyme VP4

### Introduction

To analyze whether the type of solubility tag impacts the solubility of VP4, the following constructs were designed: VP4-His6, His6-SUMO-VP4, PhoA-His6-VP4, PhoAE-His6-VP4, His6-MBP-VP4, His6-Fh8-VP4, His6-Fh8-noTEV-VP4, VP4-noTEV-Fh8-His6, and VP4-TEV-Fh8-His6.

The N′- to C′-terminal topological arrangement of the VP4 constructs is retained in the naming convention given to each VP4 constructs. E.g., solubility tag(s) before the word “VP4” signify that the tag was on the N′-terminal end of the construct with respect to the portion of the polynucleotide encoding VP4.

Some of the N′-terminal solubility tags tested for VP39 in the previous Examples were also tested for VP4. Additionally, some of the VP4 construct designs included the maltose binding protein (MBP) solubility/affinity tag or a TEV protease cleavage site used to remove tags after protein purification.

All the nucleotide sequences encoding the VP4 constructs were codon optimized by either Method A or Method B.

### Methods

Transformation and generation of glycerol stocks. The plasmids were used to transform competent cells following the manufacturer's instructions. Briefly, competent E. coli cell stocks (Arctic Express (DE3), BL21 (DE3), Shuffle), stored in a −80° C. freezer, were thawed on ice and transferred to BD Falcon round bottom tube on ice. To increase transformation efficiency, β-mercaptoethanol was diluted 1:10 with dH2O and 2 μl was added to competent E. coli cells prior to the transformation procedure. Subsequently, cells were incubated on ice for 10 min. Next, 5 ng plasmid (1 μl of 5 ng/μl stock) were added to cells and incubated on ice 30 min. Cells were then heat pulsed in a 42° C. water bath for 20 seconds and transferred to ice for 2 min. Preheated 0.9 ml (37° C.) LB medium was added to the cell plus plasmid mix and the tube was incubated at 37° C. for 1 hour, shaking at 220 rpm. 200 μl of each transformation reaction was spread on LB plates and incubated at 37° C. overnight. The next day, 3 colonies were picked from each transformation and inoculated into 1 ml of LB media and grown in deep well 96-well plate. Samples were incubated overnight shaking at 250 rpm at 37° C. All the media are supplemented with the appropriate antibiotics.

To generate the glycerol stock plate, 1:50 dilution of the overnight culture from the deep well 96-well plate was taken. 100 μl sterile 80% glycerol and 100 μl culture are added to wells of a clear 96 well cell culture plate. Plates are prepared and sealed with Thermo Fisher Adhesive Sealing Sheets and stored at −80° C.

Bacterial culture and induction conditions. To prepare the growth culture for enzyme expression, each clone was expanded so that each transformation had 3 clones with 3 technical replicates. Back dilute bacterial culture 1:500 was grown for at 37° C. with 220 rpm shaking until OD₆₀₀ was about 0.2. Upon induction with 50 μM-100 μM IPTG, the temperature was lowered to 14-22° C. After overnight induction, cells were harvested by centrifugation of plates at 3500 rpm for 10 min. Supernatant was decanted and cell pellets were stored at −80° C.

Evaluation of enzyme expression. 1 ml of cell sample was spun down at 4000 g for 10 min. Cell pellets were lysed using BugBuster Master Mix supplemented with protease inhibitors for 20 min at room temperature. The whole cell lysate sample was then spun down and pelleted for 10 min at 16000 g. Protein concentration was assayed using a BCA, samples were diluted with 0.1×JESS sample buffer to approximately 100 μg/ml. JESS standard pack reagents were prepared as described by the manufacturer's instructions. Samples were mixed with 5× fluorescent master mix and denatured at 95° C. for 5 min. Samples were loaded onto an assay plate as suggested by the manufacturer using a 1/10 dilution of a primary anti-his tag antibody and 1/20 of a secondary fluorescently labelled antibody. Assay plates were spun for 5 min at 2500 g and loaded into JESS instrument.

### Results

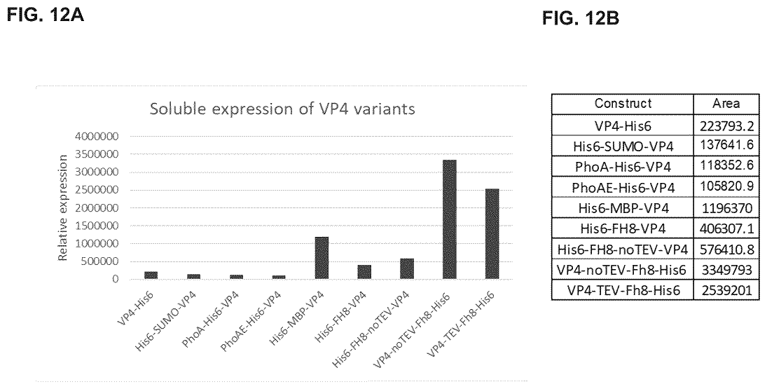

All the VP4 constructs were first screened for expression in the whole cell. All the constructs and strains showed a high whole cell expression (data not shown) and were further evaluated for soluble expression. The soluble expression screening results are summarized in FIG. 12A-FIG. 12B.

Soluble expression for the VP4 constructs was low in Shuffle strains but better in BL21 (DE3) or Arctic Express (DE3) (data not shown). Majority of the experiments were carried out in BL21 (DE3) strain.

As shown in FIG. 12, VP4 constructs with SUMO-tag or periplasmic expression tags PhoA and PhoAE did not show improved soluble expression of VP4. The VP4 constructs N-terminally tagged with Fh8 or MBP showed improved soluble expression pattern compared to an untagged VP4 (VP4-His6). The C-terminal tagged VP4 constructs with Fh8 demonstrated high soluble expression of VP4.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

### SEQUENCES

TABLE 1
Amino acid sequences

| SEQ ID NO/ | |
|---|---|
| Description | SEQUENCE |
| SEQ ID NO: | MDANVVSSSTIATYIDALAKNASELEQRSTAYEINNELELVFIKPPLITLTNVVNISTIQES |
| 1 | FIRFTVTNKEGVKIRTKIPLSKVHGLDVKNVQLVDAIDNIVWEKKSLVTENRLHKECLLRL |
| D1 | STEERHIFLDYKKYGSSIRLELVNLIQAKTKNFTIDFKLKYFLGSGAQSKSSLLHAINHPK |
| | SRPNTSLEIEFTPRDNETVPYDELIKELTTLSRHIFMASPENVILSPPINAPIKTFMLPKQD |
| | IVGLDLENLYAVTKTDGIPITIRVTSNGLYCYFTHLGYIIRYPVKRIIDSEVVVFGEAVKDK |
| | NWTVYLIKLIEPVNAINDRLEESKYVESKLVDICDRIVFKSKKYEGPFTTTSEVVDMLSTY |
| | LPKQPEGVILFYSKGPKSNIDFKIKKENTIDQTANVVFRYMSSEPIIFGESSIFVEYKKFS |
| | NDKGFPKEYGSGKIVLYNGVNYLNNIYCLEYINTHNEVGIKSVVVPIKFIAEFLVNGEILK |
| | PRIDKTMKYINSEDYYGNQHNIIVEHLRDQSIKIGDIFNEDKLSDVGHQYANNDKFRLNP |
| | EVSYFTNKRTRGPLGILSNYVKTLLISMYCSKTFLDDSNKRKVLAIDFGNGADLEKYFYG |
| | EIALLVATDPDADAIARGNERYNKLNSGIKTKYYKFDYIQETIRSDTFVSSVREVFYFGKF |
| | NIIDWQFAIHYSFHPRHYATVMNNLSELTASGGKVLITTMDGDKLSKLTDKKTFIIHKNLP |
| | SSENYMSVEKIADDRIVVYNPSTMSTPMTEYIIKKNDIVRVFNEYGFVLVDNVDFATIIER |
| | SKKFINGASTMEDRPSTRNFFELNRGAIKCEGLDVEDLLSYYVVYVFSKR |
| SEQ ID NO: | MDEIVKNIREGTHVLLPFYETLPELNLSLGKSPLPSLEYGANYFLQISRVNDLNRMPTDM |
| 2, | LKLFTHDIMLPESDLDKVYEILKINSVKYYGRSTKADAVVADLSARNKLFKRERDAIKSN |
| D12 | NHLTENNLYISDYKMLTFDVFRPLFDFVNEKYCIIKLPTLFGRGVIDTMRIYCSLFKNVRL |
| | LKCVSDSWLKDSAIMVASDVCKKNLDLFMSHVKSVTKSSSWKDVNSVQFSILNNPVDT |
| | EFINKFLEFSNRVYEALYYVHSLLYSSMTSDSKSIENKHQRRLVKLLL |
| SEQ ID NO: | MPSVQEVEKLLHVLDRNGDGKVSAEELKAFADDSKCPLDSNKIKAFIKEHDKNKDGKL |
| 3 | DLKELVSILSSMDANVVSSSTIATYIDALAKNASELEQRSTAYEINNELELVFIKPPLITLT |
| Fh8-D1 | NVVNISTIQESFIRFTVTNKEGVKIRTKIPLSKVHGLDVKNVQLVDAIDNIVWEKKSLVTE |
| | NRLHKECLLRLSTEERHIFLDYKKYGSSIRLELVNLIQAKTKNFTIDFKLKYFLGSGAQSK |
| | SSLLHAINHPKSRPNTSLEIEFTPRDNETVPYDELIKELTTLSRHIFMASPENVILSPPINA |
| | PIKTFMLPKQDIVGLDLENLYAVTKTDGIPITIRVTSNGLYCYFTHLGYIIRYPVKRIIDSEV |
| | VVFGEAVKDKNWTVYLIKLIEPVNAINDRLEESKYVESKLVDICDRIVFKSKKYEGPFTTT |
| | SEVVDMLSTYLPKQPEGVILFYSKGPKSNIDFKIKKENTIDQTANVVFRYMSSEPIIFGES |
| | SIFVEYKKFSNDKGFPKEYGSGKIVLYNGVNYLNNIYCLEYINTHNEVGIKSVVVPIKFIA |
| | EFLVNGEILKPRIDKTMKYINSEDYYGNQHNIIVEHLRDQSIKIGDIFNEDKLSDVGHQYA |
| | NNDKFRLNPEVSYFTNKRTRGPLGILSNYVKTLLISMYCSKTFLDDSNKRKVLAIDFGN |
| | GADLEKYFYGEIALLVATDPDADAIARGNERYNKLNSGIKTKYYKFDYIQETIRSDTFVS |
| | SVREVFYFGKFNIIDWQFAIHYSFHPRHYATVMNNLSELTASGGKVLITTMDGDKLSKL |
| | TDKKTFIIHKNLPSSENYMSVEKIADDRIVVYNPSTMSTPMTEYIIKKNDIVRVFNEYGFV |
| | LVDNVDFATIIERSKKFINGASTMEDRPSTRNFFELNRGAIKCEGLDVEDLLSYYVVYVF |
| | SKR |
| SEQ ID NO: | MPSVQEVEKLLHVLDRNGDGKVSAEELKAFADDSKCPLDSNKIKAFIKEHDKNKDGKL |
| 4 | DLKELVSILSSMDVVSLDKPFMYFEEIDNELDYEPESANEVAKKLPYQGQLKLLLGELFF |
| Fh8-VP39 | LSKLQRHGILDGATVVYIGSAPGTHIRYLRDHFYNLGVIIKWMLIDGRHHDPILNGLRDV |
| | TLVTRFVDEEYLRSIKKQLHPSKIILISDVRSKRGGNEPSTADLLSNYALQNVMISILNPV |
| | ASSLKWRCPFPDQWIKDFYIPHGNKMLQPFAPSYSAEMRLLSIYTGENMRLTRVTKSD |
| | AVNYEKKMYYLNKIVRNKVVVNFDYPNQEYDYFHMYFMLRTVYCNKTFPTTKAKVLFL |
| | QQSIFRFLNIPTTSTEKVSHEPIQRKISSKNSMSKNRNSKRSVRSNK |
| SEQ ID NO: | MPSVQEVEKLLHVLDRNGDGKVSAEELKAFADDSKCPLDSNKIKAFIKEHDKNKDGKL |
| 5 | DLKELVSILSSMDVVSLDKPFMYFEEIDNELDYEPESANEVAKKLPYQGQLKLLLGELFF |
| Fh8-VP39- | LSKLQRHGILDGATVVYIGSAPGTHIRYLRDHFYNLGVIIKWMLIDGRHHDPILNGLRDV |
| C26 | TLVTRFVDEEYLRSIKKQLHPSKIILISDVRSKRGGNEPSTADLLSNYALQNVMISILNPV |
| | ASSLKWRCPFPDQWIKDFYIPHGNKMLQPFAPSYSAEMRLLSIYTGENMRLTRVTKSD |
| | AVNYEKKMYYLNKIVRNKVVVNFDYPNQEYDYFHMYFMLRTVYCNKTFPTTKAKVLFL |
| | QQSIFRFLNIPTTSTEKVSHE |
| SEQ ID NO: | MDVVSLDKPFMYFEEIDNELDYEPESANEVAKKLPYQGQLKLLLGELFFLSKLQRHGIL |
| 6 | DGATVVYIGSAPGTHIRYLRDHFYNLGVIIKWMLIDGRHHDPILNGLRDVTLVTRFVDEE |
| VP39 | YLRSIKKQLHPSKIILISDVRSKRGGNEPSTADLLSNYALQNVMISILNPVASSLKWRCPF |
| | PDQWIKDFYIPHGNKMLQPFAPSYSAEMRLLSIYTGENMRLTRVTKSDAVNYEKKMYY |
| | LNKIVRNKVVVNFDYPNQEYDYFHMYFMLRTVYCNKTFPTTKAKVLFLQQSIFRFLNIPT |
| | TSTEKVSHEPIQRKISSKNSMSKNRNSKRSVRSNK |
| SEQ ID NO: | MDVVSLDKPFMYFEEIDNELDYEPESANEVAKKLPYQGQLKLLLGELFFLSKLQRHGIL |
| 7 | DGATVVYIGSAPGTHIRYLRDHFYNLGVIIKWMLIDGRHHDPILNGLRDVTLVTRFVDEE |
| VP39 C26 | YLRSIKKQLHPSKIILISDVRSKRGGNEPSTADLLSNYALQNVMISILNPVASSLKWRCPF |
| | PDQWIKDFYIPHGNKMLQPFAPSYSAEMRLLSIYTGENMRLTRVTKSDAVNYEKKMYY |
| | LNKIVRNKVVVNFDYPNQEYDYFHMYFMLRTVYCNKTFPTTKAKVLFLQQSIFRFLNIPT |
| | TSTEKVSHE |
| SEQ ID NO: | MSPILGYWKIKGLVQPTRLLLEYLEEKYEEHLYERDEGDKWRNKKFELGLEFPNLPYYI |
| 8, GST | DGDVKLTQSMAIIRYIADKHNMLGGCPKERAEISMLEGAVLDIRYGVSRIAYSKDFETLK |
| | VDFLSKLPEMLKMFEDRLCHKTYLNGDHVTHPDFMLYDALDVVLYMDPMCLDAFPKLV |
| | CFKKRIEAIPQIDKYLKSSKYIAWPLQGWQATFGGGDHPPK |
| SEQ ID NO: | EEKPKEGVKTENDHINLKVAGQDGSVVQFKIKRHTPLSKLMKAYCERQGLSMRQIRFR |
| 9, SUMO | FDGQPINETDTPAQLEMEDEDTIDVFQQQTGG |
| SEQ ID NO: | MPSVQEVEKLLHVLDRNGDGKVSAEELKAFADDSKCPLDSNKIKAFIKEHDKNKDGKL |
| 10, Fh8 | DLKELVSILSS |
| SEQ ID NO: | MMITLRKLPLAVAVAAGVMSAQAMA |
| 11, lamB | |
| SEQ ID NO: | MFKFKKKFLVGLTAAFMSISMFSATASA |
| 12, xynA | |
| SEQ ID NO: | MKQSTIALALLPLLFTPVTKA |
| 13, phoA | |
| SEQ ID NO: | MKYLLPAAAGLLLLAAQPAMA |
| 14, pelB | |
| SEQ ID NO: | MKIKTGARILALSALTTMMFSASALA |
| 15, MBP | |
| (malE gene) | |
| SEQ ID NO: | MPEPHAVLYVTNELSHIVKNGFLPIWKLTGDESLNDLWLENGKYATDVYAYGDVSKWT |
| 16 | IRQLRGHGFIFISTHKNVQLADIIKTVDVRIPREVARSHDMKAFENEISRRRIRMRKGFG |
| VP4 | DALRNYAFKMAIEFHGSEAETLNDANPRLHKIYGMPEIPPLYMEYAEIGTRFDDEPTDE |
| | KLVSMLDYIVYSAEEVHYVGCGDLRTLMQFKKRSPGRFRRVLWHVYDPIAPECSDPNV |
| | IVHNIMVDSKKDILKHMNFLKRVERLFIWDVSSDRSQMNDHEWETTRFAEDRLGEEIAY |
| | EMGGAFSSALIKHRIPNSKDEYHCISTYLFPQPGADADMYELRNFMRLRGYSHVDRHM |
| | HPDASVTKVVSRDVRKMVELYHGRDRGRFLKKRLFEHLHIVRKNGLLHESDEPRADLF |
| | YLTNRCNMGLEPSIYEVMKKSVIATAWVGRAPLYDYDDFALPRSTVMLNGSYRDIRILD |
| | GNGAILFLMWRYPDIVKKDLTYDPAWAMNFAVSLKEPIPDPPVPDISLCRFIGLRVESSV |
| | LRVRNPTLHETADELKRMGLDLSGHLYVTLMSGAYVTDLFWWFKMILDWSAQNKEQK |
| | LRDLKRSAAEVIEWKEQMAERPWHVRNDLIRALREYKRKMGMREGASIDSWLELLRH |
| | L |
| SEQ ID NO: | MPEPHAVLYVTNELSHIVKNGFLPIWKLTGDESLNDLWLENGKYATDVYAYGDVSKWT |
| 17 | IRQLRGHGFIFISTHKNVQLADIIKTVDVRIPREVARSHDMKAFENEISRRRIRMRKGFG |
| VP4-noTEV- | DALRNYAFKMAIEFHGSEAETLNDANPRLHKIYGMPEIPPLYMEYAEIGTRFDDEPTDE |
| Fh8 | KLVSMLDYIVYSAEEVHYVGCGDLRTLMQFKKRSPGRFRRVLWHVYDPIAPECSDPNV |
| | IVHNIMVDSKKDILKHMNFLKRVERLFIWDVSSDRSQMNDHEWETTRFAEDRLGEEIAY |
| | EMGGAFSSALIKHRIPNSKDEYHCISTYLFPQPGADADMYELRNFMRLRGYSHVDRHM |
| | HPDASVTKVVSRDVRKMVELYHGRDRGRFLKKRLFEHLHIVRKNGLLHESDEPRADLF |
| | YLTNRCNMGLEPSIYEVMKKSVIATAWVGRAPLYDYDDFALPRSTVMLNGSYRDIRILD |
| | GNGAILFLMWRYPDIVKKDLTYDPAWAMNFAVSLKEPIPDPPVPDISLCRFIGLRVESSV |
| | LRVRNPTLHETADELKRMGLDLSGHLYVTLMSGAYVTDLFWWFKMILDWSAQNKEQK |
| | LRDLKRSAAEVIEWKEQMAERPWHVRNDLIRALREYKRKMGMREGASIDSWLELLRH |
| | LMSDYDIDTTMPSVQEVEKLLHVLDRNGDGKVSAEELKAFADDSKCPLDSNKIKAFIKE |
| | HDKNKDGKLDLKELVSILSS |
| SEQ ID NO: | MPEPHAVLYVTNELSHIVKNGFLPIWKLTGDESLNDLWLENGKYATDVYAYGDVSKWT |
| 18 | IRQLRGHGFIFISTHKNVQLADIIKTVDVRIPREVARSHDMKAFENEISRRRIRMRKGFG |
| VP4-TEV- | DALRNYAFKMAIEFHGSEAETLNDANPRLHKIYGMPEIPPLYMEYAEIGTRFDDEPTDE |
| Fh8 | KLVSMLDYIVYSAEEVHYVGCGDLRTLMQFKKRSPGRFRRVLWHVYDPIAPECSDPNV |
| | IVHNIMVDSKKDILKHMNFLKRVERLFIWDVSSDRSQMNDHEWETTRFAEDRLGEEIAY |
| | EMGGAFSSALIKHRIPNSKDEYHCISTYLFPQPGADADMYELRNFMRLRGYSHVDRHM |
| | HPDASVTKVVSRDVRKMVELYHGRDRGRFLKKRLFEHLHIVRKNGLLHESDEPRADLF |
| | YLTNRCNMGLEPSIYEVMKKSVIATAWVGRAPLYDYDDFALPRSTVMLNGSYRDIRILD |
| | GNGAILFLMWRYPDIVKKDLTYDPAWAMNFAVSLKEPIPDPPVPDISLCRFIGLRVESSV |
| | LRVRNPTLHETADELKRMGLDLSGHLYVTLMSGAYVTDLFWWFKMILDWSAQNKEQK |
| | LRDLKRSAAEVIEWKEQMAERPWHVRNDLIRALREYKRKMGMREGASIDSWLELLRH |
| | LPMSDYDIPTTENLYFQGAMPSVQEVEKLLHVLDRNGDGKVSAEELKAFADDSKCPLD |
| | SNKIKAFIKEHDKNKDGKLDLKELVSILSS |
| SEQ ID NO: | MKHHHHHHIEEGKLVIWINGDKGYNGLAEVGKKFEKDTGIKVTVEHPDKLEEKFPQVA |
| 19 | ATGDGPDIIFWAHDRFGGYAQSGLLAEITPDKAFQDKLYPFTWDAVRYNGKLIAYPIAV |
| His6-MBP- | EALSLIYNKDLLPNPPKTWEEIPALDKELKAKGKSALMFNLQEPYFTWPLIAADGGYAFK |
| VP4 | YENGKYDIKDVGVDNAGAKAGLTFLVDLIKNKHMNADTDYSIAEAAFNKGETAMTINGP |
| | WAWSNIDTSKVNYGVTVLPTFKGQPSKPFVGVLSAGINAASPNKELAKEFLENYLLTDE |
| | GLEAVNKDKPLGAVALKSYEEELAKDPRIAATMENAQKGEIMPNIPQMSAFWYAVRTA |
| | VINAASGRQTVDEALKDAQTPMSDYDIPTTENLYFQGAMPEPHAVLYVTNELSHIVKDG |
| | FLPIWKLTGDESLNDLWLENGKYATDVYAYGDVSKWTIRQLRGHGFIFISTHKNVQLAD |
| | IIKTVDVRIPREVARSHDMKAFENEIGRRRIRMRKGFGDALRNYAFKMAIEFHGSEAETL |
| | NDANPRLHKIYGMPEIPPLYMEYAEIGTRFDDEPTDEKLVSMLDYIVYSAEEVHYIGCG |
| | DLRTLMQFKKRSPGRFRRVLWHVYDPIAPECSDPNVIVHNIMVDSKKDILKHMNFLKRV |
| | ERLFIWDVSSDRSQMNDHEWETTRFAEDRLGEEIAYEMGGAFSSALIKHRIPNSKDEY |
| | HCISTYLFPQPGADADMYELRNFMRLRGYSHVDRHMHPDASVTKVVSRDVRKMVELY |
| | HGRDRGRFLKKRLFEHLHIVRKNGLLHESDEPRADLFYLTNRCNMGLEPSIYEVMKKS |
| | VIATAWVGRAPLYDYDDFALPRSTVMLNGSYRDIRILDGNGAILFLMWRYPDIVKKDLT |
| | YDPAWAMNFAVSLKEPIPDPPVPDISLCRFIGLRVESSVLRVRNPTLHETADELKRMGL |
| | DLSGHLYVTLMSGAYVTDLFWWFKMILDWSAQNREQKLRDLKRSAAEVIEWKEQMAE |
| | RPWHVRNDLIAALREYKRKMGMREGASIDSWLELLRHL |
| SEQ ID NO: | MKHHHHHHGSLQEEKPKEGVKTENDHINLKVAGQDGSVVQFKIKRHTPLSKLMKAYC |
| 20 | ERQGLSMRQIRFRFDGQPINETDTPAQLEMEDEDTIDVFQQQTGGPMSDYDIPTTENL |
| His6-SUMO- | YFQGAMPEPHAVLYVTNELSHIVKDGFLPIWKLTGDESLNDLWLENGKYATDVYAYGD |
| VP4 | VSKWTIRQLRGHGFIFISTHKNVQLADIIKTVDVRIPREVARSHDMKAFENEIGRRRIRM |
| | RKGFGDALRNYAFKMAIEFHGSEAETLNDANPRLHKIYGMPEIPPLYMEYAEIGTRFDD |
| | EPTDEKLVSMLDYIVYSAEEVHYIGCGDLRTLMQFKKRSPGRFRRVLWHVYDPIAPEC |
| | SDPNVIVHNIMVDSKKDILKHMNFLKRVERLFIWDVSSDRSQMNDHEWETTRFAEDRL |
| | GEEIAYEMGGAFSSALIKHRIPNSKDEYHCISTYLFPQPGADADMYELRNFMRLRGYSH |
| | VDRHMHPDASVTKVVSRDVRKMVELYHGRDRGRFLKKRLFEHLHIVRKNGLLHESDE |
| | PRADLFYLTNRCNMGLEPSIYEVMKKSVIATAWVGRAPLYDYDDFALPRSTVMLNGSY |
| | RDIRILDGNGAILFLMWRYPDIVKKDLTYDPAWAMNFAVSLKEPIPDPPVPDISLCRFIGL |
| | RVESSVLRVRNPTLHETADELKRMGLDLSGHLYVTLMSGAYVTDLFWWFKMILDWSA |
| | QNREQKLRDLKRSAAEVIEWKEQMAERPWHVRNDLIAALREYKRKMGMREGASIDSW |
| | LELLRHL |
| SEQ ID NO: | MPEPHAVLYVTNELSHIVKNGFLPIWKLTGDESLNDLWLENGKYATDVYAYGDVSKWT |
| 21 | IRQLRGHGFIFISTHKNVQLADIIKTVDVRIPREVARSHDMKAFENEISRRRIRMRKGFG |
| VP4-noTEV- | DALRNYAFKMAIEFHGSEAETLNDANPRLHKIYGMPEIPPLYMEYAEIGTRFDDEPTDE |
| Fh8-His6 | KLVSMLDYIVYSAEEVHYVGCGDLRTLMQFKKRSPGRFRRVLWHVYDPIAPECSDPNV |
| | IVHNIMVDSKKDILKHMNFLKRVERLFIWDVSSDRSQMNDHEWETTRFAEDRLGEEIAY |
| | EMGGAFSSALIKHRIPNSKDEYHCISTYLFPQPGADADMYELRNFMRLRGYSHVDRHM |
| | HPDASVTKVVSRDVRKMVELYHGRDRGRFLKKRLFEHLHIVRKNGLLHESDEPRADLF |
| | YLTNRCNMGLEPSIYEVMKKSVIATAWVGRAPLYDYDDFALPRSTVMLNGSYRDIRILD |
| | GNGAILFLMWRYPDIVKKDLTYDPAWAMNFAVSLKEPIPDPPVPDISLCRFIGLRVESSV |
| | LRVRNPTLHETADELKRMGLDLSGHLYVTLMSGAYVTDLFWWFKMILDWSAQNKEQK |
| | LRDLKRSAAEVIEWKEQMAERPWHVRNDLIRALREYKRKMGMREGASIDSWLELLRH |
| | LMSDYDIDTTMPSVQEVEKLLHVLDRNGDGKVSAEELKAFADDSKCPLDSNKIKAFIKE |
| | HDKNKDGKLDLKELVSILSSMKHHHHHH |
| SEQ ID NO: | MPSVQEVEKLLHVLDRNGDGKVSAEELKAFADDSKCPLDSNKIKAFIKEHDKNKDGKL |
| 22 | DLKELVSILSSMPEPHAVLYVTNELSHIVKDGFLPIWKLTGDESLNDLWLENGKYATDV |
| Fh8-noTEV- | YAYGDVSKWTIRQLRGHGFIFISTHKNVQLADIIKTVDVRIPREVARSHDMKAFENEIGR |
| VP4 | RRIRMRKGFGDALRNYAFKMAIEFHGSEAETLNDANPRLHKIYGMPEIPPLYMEYAEIG |
| | TRFDDEPTDEKLVSMLDYIVYSAEEVHYIGCGDLRTLMQFKKRSPGRFRRVLWHVYDP |
| | IAPECSDPNVIVHNIMVDSKKDILKHMNFLKRVERLFIWDVSSDRSQMNDHEWETTRFA |
| | EDRLGEEIAYEMGGAFSSALIKHRIPNSKDEYHCISTYLFPQPGADADMYELRNFMRLR |
| | GYSHVDRHMHPDASVTKVVSRDVRKMVELYHGRDRGRFLKKRLFEHLHIVRKNGLLH |
| | ESDEPRADLFYLTNRCNMGLEPSIYEVMKKSVIATAWVGRAPLYDYDDFALPRSTVML |
| | NGSYRDIRILDGNGAILFLMWRYPDIVKKDLTYDPAWAMNFAVSLKEPIPDPPVPDISLC |
| | RFIGLRVESSVLRVRNPTLHETADELKRMGLDLSGHLYVTLMSGAYVTDLFWWFKMIL |
| | DWSAQNREQKLRDLKRSAAEVIEWKEQMAERPWHVRNDLIAALREYKRKMGMREGA |
| | SIDSWLELLRHL |
| SEQ ID NO: | MKQSTIALALLPLLFTPVTKAMKHHHHHHPMSDYDIPTTENLYFQGAMPEPHAVLYVTN |
| 43 | ELSHIVKDGFLPIWKLTGDESLNDLWLENGKYATDVYAYGDVSKWTIRQLRGHGFIFIS |
| PhoAE- | THKNVQLADIIKTVDVRIPREVARSHDMKAFENEIGRRRIRMRKGFGDALRNYAFKMAI |
| His6-VP4 | EFHGSEAETLNDANPRLHKIYGMPEIPPLYMEYAEIGTRFDDEPTDEKLVSMLDYIVYSA |
| | EEVHYIGCGDLRTLMQFKKRSPGRFRRVLWHVYDPIAPECSDPNVIVHNIMVDSKKDIL |
| | KHMNFLKRVERLFIWDVSSDRSQMNDHEWETTRFAEDRLGEEIAYEMGGAFSSALIKH |
| | RIPNSKDEYHCISTYLFPQPGADADMYELRNFMRLRGYSHVDRHMHPDASVTKVVSR |
| | DVRKMVELYHGRDRGRFLKKRLFEHLHIVRKNGLLHESDEPRADLFYLTNRCNMGLEP |
| | SIYEVMKKSVIATAWVGRAPLYDYDDFALPRSTVMLNGSYRDIRILDGNGAILFLMWRY |
| | PDIVKKDLTYDPAWAMNFAVSLKEPIPDPPVPDISLCRFIGLRVESSVLRVRNPTLHETA |
| | DELKRMGLDLSGHLYVTLMSGAYVTDLFWWFKMILDWSAQNREQKLRDLKRSAAEVI |
| | EWKEQMAERPWHVRNDLIAALREYKRKMGMREGASIDSWLELLRHL |

TABLE 2
Nucleotide sequences

| SEQ ID NO/ | |
|---|---|
| Description | SEQUENCE |
| SEQ ID NO: | ATGAAACATCACCATCACCATCACATGCCGTCCGTTCAAGAAGTCGAAAAACTTCT |
| 23 | GCATGTTCTGGATCGTAACGGCGATGGAAAAGTATCCGCCGAGGAATTGAAGGCA |
| His6-Fh8- | TTTGCTGACGATTCGAAATGTCCGTTGGATAGTAATAAGATCAAAGCATTCATCAAA |
| V39 | GAGCATGACAAGAACAAGGACGGGAAATTGGATTTGAAGGAGTTGGTGTCTATTCT |
| VP39 | TAGCTCTCCCATGAGCGATTACGACATCCCCACTACTGAGAATCTTTATTTTCAGGG |
| sequence is | CGCCATGGACGTTGTAAGCTTAGATAAACCGTTCATGTATTTTGAGGAAATCGATAA |
| codon | CGAACTGGATTATGAACCGGAATCGGCGAATGAGGTGGCTAAAAAATTGCCATACC |
| optimized | AGGGTCAACTTAAATTATTATTGGGGGAACTGTTTTTTTTAAGTAAGCTTCAGCGTC |
| according to | ATGGGATTCTTGACGGTGCAACGGTGGTATACATTGGGTCGGCCCCTGGCACTCA |
| method A | TATCCGCTACTTACGCGACCACTTCTACAACTTAGGCGTCATCATCAAATGGATGTT |
| | GATCGATGGGCGCCATCACGACCCCATTCTTAATGGCTTGCGCGATGTAACACTTG |
| | TTACACGTTTCGTCGATGAAGAGTACTTACGTAGCATTAAAAAACAACTTCACCCAT |
| | CCAAAATCATTCTGATCTCTGACGTACGTAGCAAACGTGGGGGTAATGAACCAAGT |
| | ACAGCTGACCTTCTGTCTAACTATGCTTTACAAAACGTGATGATTTCCATCTTGAAT |
| | CCCGTTGCTTCATCATTGAAGTGGCGTTGCCCCTTCCCGGACCAGTGGATCAAAGA |
| | TTTCTACATCCCTCACGGCAACAAGATGTTGCAACCGTTCGCGCCATCCTATTCGG |
| | CCGAAATGCGCTTGTTGTCCATCTACACAGGCGAGAACATGCGTTTAACTCGTGTA |
| | ACAAAGTCGGATGCTGTAAACTACGAAAAAAAGATGTACTATCTTAATAAGATTGTT |
| | CGTAACAAAGTGGTTGTGAATTTCGATTACCCTAACCAGGAGTACGATTATTTTCAT |
| | ATGTATTTTATGTTGCGTACCGTTTATTGTAATAAGACTTTTCCGACAACTAAGGCAA |
| | AGGTGCTGTTTCTGCAGCAATCGATTTTCCGCTTCTTAAACATCCCCACCACAAGTA |
| | CAGAAAAAGTCTCACACGAGCCCATCCAACGCAAGATTTCTTCTAAAAATAGCATGA |
| | GCAAAAATCGCAATTCAAAACGCTCGGTTCGTAGTAACAAATAG |
| SEQ ID NO: | ATGAAACATCACCATCACCATCACATGCCGTCCGTTCAAGAAGTCGAAAAACTTCT |
| 24 | GCATGTTCTGGATCGTAACGGCGATGGAAAAGTATCCGCCGAGGAATTGAAGGCA |
| His6-Fh8- | TTTGCTGACGATTCGAAATGTCCGTTGGATAGTAATAAGATCAAAGCATTCATCAAA |
| D1-PT7-D12 | GAGCATGACAAGAACAAGGACGGGAAATTGGATTTGAAGGAGTTGGTGTCTATTCT |
| PT7 = T7 | TAGCTCTCCCATGAGCGATTACGACATCCCCACTACTGAGAATCTTTATTTTCAGGG |
| promoter | CGCCATGGACGCTAATGTCGTCTCGTCAAGTACGATCGCTACCTACATTGACGCCT |
| D1 and D12 | TGGCGAAAAATGCTAGCGAGCTTGAACAGCGCAGCACTGCTTATGAGATCAATAAT |
| sequences | GAGTTAGAGCTTGTATTTATCAAACCACCGTTGATTACTCTGACGAACGTAGTTAAC |
| are codon | ATTTCAACCATTCAAGAGTCCTTTATTCGTTTTACCGTCACGAACAAAGAGGGCGTT |
| optimized | AAAATCCGCACCAAGATCCCTCTTAGTAAAGTGCATGGATTAGATGTCAAAAATGTT |
| according to | CAGCTGGTAGACGCCATTGATAACATCGTATGGGAGAAGAAATCACTGGTTACTGA |
| method A | AAACCGCCTGCACAAAGAATGTCTGTTGCGTCTGTCTACCGAGGAACGTCATATTT |
| | TCCTTGACTACAAGAAATACGGTTCGAGTATTCGCTTAGAACTTGTGAACCTGATCC |
| | AGGCCAAGACTAAAAATTTTACAATTGATTTTAAATTAAAATACTTCTTGGGAAGTGG |
| | GGCACAATCCAAGTCGTCCTTGTTACATGCTATCAACCATCCCAAATCCCGTCCTAA |
| | CACAAGCTTGGAGATTGAATTCACACCGCGTGATAACGAAACTGTTCCTTATGACG |
| | AGCTGATTAAAGAATTGACGACGCTTTCTCGCCATATCTTCATGGCCTCTCCCGAAA |
| | ACGTCATCCTTAGCCCTCCAATCAATGCACCAATTAAGACATTTATGTTACCGAAGC |
| | AAGACATTGTAGGCTTGGATCTTGAAAATCTTTATGCTGTTACCAAAACAGATGGTA |
| | TTCCAATTACAATTCGTGTTACATCTAACGGCCTTTACTGCTATTTCACCCACTTGG |
| | GATATATCATCCGTTATCCGGTTAAGCGTATTATCGACTCTGAGGTAGTAGTTTTTG |
| | GCGAAGCAGTAAAGGACAAGAATTGGACAGTATACCTGATTAAATTAATTGAACCG |
| | GTAAATGCCATTAATGATCGCTTAGAGGAAAGCAAGTATGTCGAATCTAAACTTGTT |
| | GATATCTGTGATCGCATCGTATTTAAAAGTAAAAAATACGAAGGGCCTTTTACTACA |
| | ACAAGTGAAGTTGTTGACATGTTATCTACCTATTTACCAAAACAACCTGAAGGTGTT |
| | ATTTTGTTCTATTCCAAGGGCCCCAAATCGAACATTGACTTTAAGATCAAGAAGGAA |
| | AACACTATTGATCAGACCGCCAACGTAGTATTCCGCTATATGTCCAGTGAACCGATT |
| | ATTTTCGGCGAGAGTAGCATTTTCGTTGAATATAAAAAATTTTCCAACGACAAGGGC |
| | TTCCCGAAAGAGTATGGGTCAGGCAAGATTGTATTGTATAATGGTGTCAATTATOTT |
| | AATAACATCTATTGCCTTGAGTATATCAATACGCACAACGAGGTGGGCATTAAGTCC |
| | GTAGTAGTGCCCATCAAGTTCATCGCTGAGTTCTTGGTGAACGGGGAGATCTTAAA |
| | ACCTCGTATCGACAAGACTATGAAATATATCAACTCCGAAGACTATTATGGGAATCA |
| | GCACAACATCATTGTAGAGCACCTGCGCGACCAGTCCATTAAGATTGGAGATATTT |
| | TTAACGAAGATAAGCTGTCCGACGTCGGGCACCAGTACGCGAACAACGATAAGTTC |
| | CGTTTGAATCCTGAAGTATCATATTTCACAAACAAACGCACACGTGGTCCACTGGG |
| | GATCTTGTCGAACTATGTCAAAACGCTGTTAATCTCTATGTATTGTTCAAAGACTTTC |
| | CTTGATGATAGCAACAAGCGTAAGGTACTGGCTATCGATTTTGGAAACGGCGCCGA |
| | CTTGGAGAAATACTTTTACGGCGAAATCGCTCTGTTAGTTGCTACAGATCCAGACG |
| | CAGATGCGATCGCTCGCGGTAACGAGCGCTATAATAAACTTAACTCCGGCATTAAA |
| | ACTAAGTATTACAAGTTCGACTACATCCAAGAGACTATTCGTAGTGATACGTTCGTG |
| | AGTTCGGTGCGCGAGGTATTCTATTTCGGTAAATTTAATATCATCGACTGGCAGTTT |
| | GCCATCCATTATAGTTTCCATCCACGCCACTATGCCACAGTTATGAATAACTTAAGT |
| | GAGCTTACAGCAAGCGGTGGTAAAGTGCTGATTACCACAATGGACGGCGATAAGTT |
| | GTCTAAGTTGACGGACAAAAAAACATTCATTATCCATAAGAATCTGCCTTCAAGTGA |
| | AAATTATATGTCAGTAGAGAAGATCGCCGACGACCGCATCGTAGTTTACAATCCGA |
| | GTACCATGAGTACTCCCATGACGGAGTACATCATCAAAAAGAATGACATCGTCCGT |
| | GTTTTCAATGAGTACGGCTTCGTACTTGTAGATAACGTCGATTTTGCAACGATTATT |
| | GAACGCTCAAAGAAGTTTATTAATGGGGCGTCAACAATGGAGGATCGCCCTTCCAC |
| | TCGCAATTTCTTTGAATTAAATCGTGGGGCCATCAAGTGCGAGGGCCTGGATGTAG |
| | AAGATTTATTGTCATATTACGTCGTGTACGTTTTCTCCAAGCGTTAAGCTAGCAGGA |
| | TCCGAATTCGAGCTCGGCGCGCCTGCAGGTCGACAAGCTTGCGGCCGCATAATGC |
| | TTAAGTCGAACAGAAAGTAATCGTATTGTACACGGCCGCATAATCGAAATTAATACG |
| | ACTCACTATAGGGGAATTGTGAGCGGATAACAATTCCCCATCTTAGTATATTAGTTA |
| | AGTATAAGAAGGAGATATACATATGGACGAGATTGTAAAGAACATTCGTGAAGGAA |
| | CGCACGTGTTGCTTCCGTTCTACGAGACCTTACCAGAGTTGAACTTATCGCTTGGG |
| | AAATCCCCGTTGCCTTCGCTTGAGTATGGTGCAAACTATTTCCTGCAAATTAGTCGC |
| | GTAAATGATCTTAACCGTATGCCGACGGACATGCTGAAATTATTTACCCACGATATT |
| | ATGTTACCCGAATCAGATCTGGACAAAGTATATGAGATTCTTAAAATCAACTCTGTG |
| | AAATACTACGGCCGTAGCACGAAAGCCGACGCGGTCGTCGCAGACCTTTCTGCGC |
| | GCAACAAGCTTTTCAAGCGCGAGCGCGACGCGATTAAATCTAACAACCATTTGACC |
| | GAAAACAACCTTTACATCAGCGACTATAAGATGCTGACCTTTGACGTATTCCGCCC |
| | GTTGTTCGACTTCGTGAATGAAAAATATTGCATTATTAAATTACCAACGTTGTTTGGC |
| | CGTGGCGTAATCGACACCATGCGTATTTACTGTTCCTTGTTTAAAAATGTTCGCTTG |
| | CTTAAATGCGTTTCTGATAGCTGGTTGAAGGATTCTGCGATCATGGTCGCTTCCGA |
| | TGTATGTAAAAAAAACTTGGACTTGTTCATGTCGCATGTTAAATCGGTTACTAAATCT |
| | AGCTCATGGAAGGATGTCAATTCCGTTCAGTTCTCAATTCTTAACAACCCCGTAGAT |
| | ACGGAGTTCATCAACAAATTTTTAGAGTTCTCCAACCGCGTGTATGAGGCACTGTA |
| | CTACGTTCATTCTTTATTATACTCTAGCATGACCAGTGATTCAAAAAGCATTGAAAAC |
| | AAACATCAGCGCCGCCTGGTTAAGCTTCTGTTATAA |
| SEQ ID NO: | TGGCGAATGGGACGCGCCCTGTAGCGGCGCATTAAGCGCGGCGGGTGTGGTGGT |
| 25 | TACGCGCAGCGTGACCGCTACACTTGCCAGCGCCCTAGCGCCCGCTCCTTTCGCT |
| pET-28a | TTCTTCCCTTCCTTTCTCGCCACGTTCGCCGGCTTTCCCCGTCAAGCTCTAAATCG |
| His6-GST- | GGGGCTCCCTTTAGGGTTCCGATTTAGTGCTTTACGGCACCTCGACCCCAAAAAAC |
| V39 C26 | TTGATTAGGGTGATGGTTCACGTAGTGGGCCATCGCCCTGATAGACGGTTTTTCGC |
| VP39-C26 | CCTTTGACGTTGGAGTCCACGTTCTTTAATAGTGGACTCTTGTTCCAAACTGGAACA |
| sequence is | ACACTCAACCCTATCTCGGTCTATTCTTTTGATTTATAAGGGATTTTGCCGATTTCG |
| codon | GCCTATTGGTTAAAAAATGAGCTGATTTAACAAAAATTTAACGCGAATTTTAACAAAA |
| optimized | TATTAACGTTTACAATTTCAGGTGGCACTTTTCGGGGAAATGTGCGCGGAACCCCT |
| according to | ATTTGTTTATTTTTCTAAATACATTCAAATATGTATCCGCTCATGAATTAATTCTTAGA |
| method B | AAAACTCATCGAGCATCAAATGAAACTGCAATTTATTCATATCAGGATTATCAATACC |
| | ATATTTTTGAAAAAGCCGTTTCTGTAATGAAGGAGAAAACTCACCGAGGCAGTTCCA |
| | TAGGATGGCAAGATCCTGGTATCGGTCTGCGATTCCGACTCGTCCAACATCAATAC |
| | AACCTATTAATTTCCCCTCGTCAAAAATAAGGTTATCAAGTGAGAAATCACCATGAG |
| | TGACGACTGAATCCGGTGAGAATGGCAAAAGTTTATGCATTTCTTTCCAGACTTGTT |
| | CAACAGGCCAGCCATTACGCTCGTCATCAAAATCACTCGCATCAACCAAACCGTTA |
| | TTCATTCGTGATTGCGCCTGAGCGAGACGAAATACGCGATCGCTGTTAAAAGGACA |
| | ATTACAAACAGGAATCGAATGCAACCGGCGCAGGAACACTGCCAGCGCATCAACA |
| | ATATTTTCACCTGAATCAGGATATTCTTCTAATACCTGGAATGCTGTTTTCCCGGGG |
| | ATCGCAGTGGTGAGTAACCATGCATCATCAGGAGTACGGATAAAATGCTTGATGGT |
| | CGGAAGAGGCATAAATTCCGTCAGCCAGTTTAGTCTGACCATCTCATCTGTAACAT |
| | CATTGGCAACGCTACCTTTGCCATGTTTCAGAAACAACTCTGGCGCATCGGGCTTC |
| | CCATACAATCGATAGATTGTCGCACCTGATTGCCCGACATTATCGCGAGCCCATTT |
| | ATACCCATATAAATCAGCATCCATGTTGGAATTTAATCGCGGCCTAGAGCAAGACG |
| | TTTCCCGTTGAATATGGCTCATAACACCCCTTGTATTACTGTTTATGTAAGCAGACA |
| | GTTTTATTGTTCATGACCAAAATCCCTTAACGTGAGTTTTCGTTCCACTGAGCGTCA |
| | GACCCCGTAGAAAAGATCAAAGGATCTTCTTGAGATCCTTTTTTTCTGCGCGTAATC |
| | TGCTGCTTGCAAACAAAAAAACCACCGCTACCAGCGGTGGTTTGTTTGCCGGATCA |
| | AGAGCTACCAACTCTTTTTCCGAAGGTAACTGGCTTCAGCAGAGCGCAGATACCAA |
| | ATACTGTCCTTCTAGTGTAGCCGTAGTTAGGCCACCACTTCAAGAACTCTGTAGCA |
| | CCGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAGC |
| | GGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGCAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCTT |
| | TTGCTCACATGTTCTTTCCTGCGTTATCCCCTGATTCTGTGGATAACCGTATTACCG |
| | CCTTTGAGTGAGCTGATACCGCTCGCCGCAGCCGAACGACCGAGCGCAGCGAGTC |
| | AGTGAGCGAGGAAGCGGAAGAGCGCCTGATGCGGTATTTTCTCCTTACGCATCTG |
| | TGCGGTATTTCACACCGCATATATGGTGCACTCTCAGTACAATCTGCTCTGATGCC |
| | GCATAGTTAAGCCAGTATACACTCCGCTATCGCTACGTGACTGGGTCATGGCTGCG |
| | CCCCGACACCCGCCAACACCCGCTGACGCGCCCTGACGGGCTTGTCTGCTCCCG |
| | GCATCCGCTTACAGACAAGCTGTGACCGTCTCCGGGAGCTGCATGTGTCAGAGGT |
| | TTTCACCGTCATCACCGAAACGCGCGAGGCAGCTGCGGTAAAGCTCATCAGCGTG |
| | GTCGTGAAGCGATTCACAGATGTCTGCCTGTTCATCCGCGTCCAGCTCGTTGAGTT |
| | TCTCCAGAAGCGTTAATGTCTGGCTTCTGATAAAGCGGGCCATGTTAAGGGCGGTT |
| | TTTTCCTGTTTGGTCACTGATGCCTCCGTGTAAGGGGGATTTCTGTTCATGGGGGT |
| | AATGATACCGATGAAACGAGAGAGGATGCTCACGATACGGGTTACTGATGATGAAC |
| | ATGCCCGGTTACTGGAACGTTGTGAGGGTAAACAACTGGCGGTATGGATGCGGCG |
| | GGACCAGAGAAAAATCACTCAGGGTCAATGCCAGCGCTTCGTTAATACAGATGTAG |
| | GTGTTCCACAGGGTAGCCAGCAGCATCCTGCGATGCAGATCCGGAACATAATGGT |
| | GCAGGGCGCTGACTTCCGCGTTTCCAGACTTTACGAAACACGGAAACCGAAGACC |
| | ATTCATGTTGTTGCTCAGGTCGCAGACGTTTTGCAGCAGCAGTCGCTTCACGTTCG |
| | CTCGCGTATCGGTGATTCATTCTGCTAACCAGTAAGGCAACCCCGCCAGCCTAGCC |
| | GGGTCCTCAACGACAGGAGCACGATCATGCGCACCCGTGGGGCCGCCATGCCGG |
| | CGATAATGGCCTGCTTCTCGCCGAAACGTTTGGTGGCGGGACCAGTGACGAAGGC |
| | TTGAGCGAGGGCGTGCAAGATTCCGAATACCGCAAGCGACAGGCCGATCATCGTC |
| | GCGCTCCAGCGAAAGCGGTCCTCGCCGAAAATGACCCAGAGCGCTGCCGGCACC |
| | TGTCCTACGAGTTGCATGATAAAGAAGACAGTCATAAGTGCGGCGACGATAGTCAT |
| | GCCCCGCGCCCACCGGAAGGAGCTGACTGGGTTGAAGGCTCTCAAGGGCATCGG |
| | TCGAGATCCCGGTGCCTAATGAGTGAGCTAACTTACATTAATTGCGTTGCGCTCAC |
| | TGCCCGCTTTCCAGTCGGGAAACCTGTCGTGCCAGCTGCATTAATGAATCGGCCAA |
| | CGCGCGGGGAGAGGCGGTTTGCGTATTGGGCGCCAGGGTGGTTTTTCTTTTCACC |
| | AGTGAGACGGGCAACAGCTGATTGCCCTTCACCGCCTGGCCCTGAGAGAGTTGCA |
| | GCAAGCGGTCCACGCTGGTTTGCCCCAGCAGGCGAAAATCCTGTTTGATGGTGGT |
| | TAACGGGGGGATATAACATGAGCTGTCTTCGGTATCGTCGTATCCCACTACCGAGA |
| | TATCCGCACCAACGCGCAGCCCGGACTCGGTAATGGCGCGCATTGCGCCCAGCG |
| | CCATCTGATCGTTGGCAACCAGCATCGCAGTGGGAACGATGCCCTCATTCAGCATT |
| | TGCATGGTTTGTTGAAAACCGGACATGGCACTCCAGTCGCCTTCCCGTTCCGCTAT |
| | CGGCTGAATTTGATTGCGAGTGAGATATTTATGCCAGCCAGCCAGACGCAGACGC |
| | GCCGAGACAGAACTTAATGGGCCCGCTAACAGCGCGATTTGCTGGTGACCCAATG |
| | CGACCAGATGCTCCACGCCCAGTCGCGTACCGTCTTCATGGGAGAAAATAATACTG |
| | TTGATGGGTGTCTGGTCAGAGACATCAAGAAATAACGCCGGAACATTAGTGCAGGC |
| | AGCTTCCACAGCAATGGCATCCTGGTCATCCAGCGGATAGTTAATGATCAGCCCAC |
| | TGACGCGTTGCGCGAGAAGATTGTGCACCGCCGCTTTACAGGCTTCGACGCCGCT |
| | TCGTTCTACCATCGACACCACCACGCTGGCACCCAGTTGATCGGCGCGAGATTTAA |
| | TCGCCGCGACAATTTGCGACGGCGCGTGCAGGGCCAGACTGGAGGTGGCAACGC |
| | CAATCAGCAACGACTGTTTGCCCGCCAGTTGTTGTGCCACGCGGTTGGGAATGTAA |
| | TTCAGCTCCGCCATCGCCGCTTCCACTTTTTCCCGCGTTTTCGCAGAAACGTGGCT |
| | GGCCTGGTTCACCACGCGGGAAACGGTCTGATAAGAGACACCGGCATACTCTGCG |
| | ACATCGTATAACGTTACTGGTTTCACATTCACCACCCTGAATTGACTCTCTTCCGGG |
| | CGCTATCATGCCATACCGCGAAAGGTTTTGCGCCATTCGATGGTGTCCGGGATCTC |
| | GACGCTCTCCCTTATGCGACTCCTGCATTAGGAAGCAGCCCAGTAGTAGGTTGAG |
| | GCCGTTGAGCACCGCCGCCGCAAGGAATGGTGCATGCAAGGAGATGGCGCCCAA |
| | CAGTCCCCCGGCCACGGGGCCTGCCACCATACCCACGCCGAAACAAGCGCTCAT |
| | GAGCCCGAAGTGGCGAGCCCGATCTTCCCCATCGGTGATGTCGGCGATATAGGCG |
| | CCAGCAACCGCACCTGTGGCGCCGGTGATGCCGGCCACGATGCGTCCGGCGTAG |
| | AGGATCGAGATCTGAAATTAATACGACTCACTATAGGGGAATTGTGAGCGGATAAC |
| | AATTCCCCTCTAGAAATAATTTTGTTTAACTTTAAGAAGGAGATATACCATGAAACAT |
| | CACCATCACCATCACATGTCCCCTATACTAGGTTATTGGAAAATTAAGGGCCTTGTG |
| | CAACCCACTCGACTTCTTTTGGAATATCTTGAAGAAAAATATGAAGAGCATTTGTAT |
| | GAGCGCGATGAAGGTGATAAATGGCGAAACAAAAAGTTTGAATTGGGTTTGGAGTT |
| | TCCCAATCTTCCTTATTATATTGATGGTGATGTTAAATTAACACAGTCTATGGCCATC |
| | ATACGTTATATAGCTGACAAGCACAACATGTTGGGTGGTTGTCCAAAAGAGCGTGC |
| | AGAGATTTCAATGCTTGAAGGAGCGGTTTTGGATATTAGATACGGTGTTTCGAGAAT |
| | TGCATATAGTAAAGACTTTGAAACTCTCAAAGTTGATTTTCTTAGCAAGCTACCTGA |
| | AATGCTGAAAATGTTCGAAGATCGTTTATGTCATAAAACATATTTAAATGGTGATCAT |
| | GTAACCCATCCTGACTTCATGTTGTATGACGCTCTTGATGTTGTTTTATACATGGAC |
| | CCAATGTGCCTGGATGCGTTCCCAAAATTAGTTTGTTTTAAAAAACGTATTGAAGCT |
| | ATCCCACAAATTGATAAGTACTTGAAATCCAGCAAGTATATAGCATGGCCTTTGCAG |
| | GGCTGGCAAGCCACGTTTGGTGGTGGCGACCATCCTCCAAAACCCATGAGCGATT |
| | ACGACATCCCCACTACTGAGAATCTTTATTTTCAGGGCGCCATGGATGTTGTGTCG |
| | TTAGATAAACCGTTTATGTATTTTGAGGAAATTGATAATGAGTTAGATTACGAACCA |
| | GAAAGTGCAAATGAGGTCGCAAAAAAACTGCCGTATCAAGGACAGTTAAAACTATT |
| | ACTAGGAGAATTATTTTTTCTTAGTAAGTTACAGCGACACGGTATATTAGATGGTGC |
| | CACCGTAGTGTATATAGGATCTGCTCCCGGTACACATATACGTTATTTGAGAGATCA |
| | TTTCTATAATTTAGGAGTGATCATCAAATGGATGCTAATTGACGGCCGCCATCATGA |
| | TCCTATTTTAAATGGATTGCGTGATGTGACTCTAGTGACTCGGTTCGTTGATGAGGA |
| | ATATCTACGATCCATCAAAAAACAACTGCATCCTTCTAAGATTATTTTAATTTCTGAT |
| | GTGAGATCCAAACGAGGAGGAAATGAACCTAGTACGGCGGATTTACTAAGTAATTA |
| | CGCTCTACAAAATGTCATGATTAGTATTTTAAACCCCGTGGCGTCTAGTCTTAAATG |
| | GAGATGCCCGTTTCCAGATCAATGGATCAAGGACTTTTATATCCCACACGGTAATAA |
| | AATGTTACAACCTTTTGCTCCTTCATATTCAGCTGAAATGAGATTATTAAGTATTTAT |
| | ACCGGTGAGAACATGAGACTGACTCGAGTTACCAAATCAGACGCTGTAAATTATGA |
| | AAAAAAGATGTACTACCTTAATAAGATCGTCCGTAACAAAGTAGTTGTTAACTTTGAT |
| | TATCCTAATCAGGAATATGACTATTTTCACATGTACTTTATGCTGAGGACCGTGTAC |
| | TGCAATAAAACATTTCCTACTACTAAAGCAAAGGTACTATTTCTACAACAATCTATAT |
| | TTCGTTTCTTAAATATTCCAACAACATCAACTGAAAAAGTTAGTCATGAATAGCTCGA |
| | GCACCACCACCACCACCACTGAGATCCGGCTGCTAACAAAGCCCGAAAGGAAGCT |
| | GAGTTGGCTGCTGCCACCGCTGAGCAATAACTAGCATAACCCCTTGGGGCCTCTAA |
| | ACGGGTCTTGAGGGGTTTTTTGCTGAAAGGAGGAACTATATCCGGATTCCGGAT |
| SEQ ID NO: | TGGCGAATGGGACGCGCCCTGTAGCGGCGCATTAAGCGCGGCGGGTGTGGTGGT |
| 26 | TACGCGCAGCGTGACCGCTACACTTGCCAGCGCCCTAGCGCCCGCTCCTTTCGCT |
| pET-28a | TTCTTCCCTTCCTTTCTCGCCACGTTCGCCGGCTTTCCCCGTCAAGCTCTAAATCG |
| His6-GST- | GGGGCTCCCTTTAGGGTTCCGATTTAGTGCTTTACGGCACCTCGACCCCAAAAAAC |
| V39 | TTGATTAGGGTGATGGTTCACGTAGTGGGCCATCGCCCTGATAGACGGTTTTTCGC |
| VP39 | CCTTTGACGTTGGAGTCCACGTTCTTTAATAGTGGACTCTTGTTCCAAACTGGAACA |
| sequence is | ACACTCAACCCTATCTCGGTCTATTCTTTTGATTTATAAGGGATTTTGCCGATTTCG |
| codon | GCCTATTGGTTAAAAAATGAGCTGATTTAACAAAAATTTAACGCGAATTTTAACAAAA |
| optimized | TATTAACGTTTACAATTTCAGGTGGCACTTTTCGGGGAAATGTGCGCGGAACCCCT |
| according to | ATTTGTTTATTTTTCTAAATACATTCAAATATGTATCCGCTCATGAATTAATTCTTAGA |
| method A | AAAACTCATCGAGCATCAAATGAAACTGCAATTTATTCATATCAGGATTATCAATACC |
| | ATATTTTTGAAAAAGCCGTTTCTGTAATGAAGGAGAAAACTCACCGAGGCAGTTCCA |
| | TAGGATGGCAAGATCCTGGTATCGGTCTGCGATTCCGACTCGTCCAACATCAATAC |
| | AACCTATTAATTTCCCCTCGTCAAAAATAAGGTTATCAAGTGAGAAATCACCATGAG |
| | TGACGACTGAATCCGGTGAGAATGGCAAAAGTTTATGCATTTCTTTCCAGACTTGTT |
| | CAACAGGCCAGCCATTACGCTCGTCATCAAAATCACTCGCATCAACCAAACCGTTA |
| | TTCATTCGTGATTGCGCCTGAGCGAGACGAAATACGCGATCGCTGTTAAAAGGACA |
| | ATTACAAACAGGAATCGAATGCAACCGGCGCAGGAACACTGCCAGCGCATCAACA |
| | ATATTTTCACCTGAATCAGGATATTCTTCTAATACCTGGAATGCTGTTTTCCCGGGG |
| | ATCGCAGTGGTGAGTAACCATGCATCATCAGGAGTACGGATAAAATGCTTGATGGT |
| | CGGAAGAGGCATAAATTCCGTCAGCCAGTTTAGTCTGACCATCTCATCTGTAACAT |
| | CATTGGCAACGCTACCTTTGCCATGTTTCAGAAACAACTCTGGCGCATCGGGCTTC |
| | CCATACAATCGATAGATTGTCGCACCTGATTGCCCGACATTATCGCGAGCCCATTT |
| | ATACCCATATAAATCAGCATCCATGTTGGAATTTAATCGCGGCCTAGAGCAAGACG |
| | TTTCCCGTTGAATATGGCTCATAACACCCCTTGTATTACTGTTTATGTAAGCAGACA |
| | GTTTTATTGTTCATGACCAAAATCCCTTAACGTGAGTTTTCGTTCCACTGAGCGTCA |
| | GACCCCGTAGAAAAGATCAAAGGATCTTCTTGAGATCCTTTTTTTCTGCGCGTAATC |
| | TGCTGCTTGCAAACAAAAAAACCACCGCTACCAGCGGTGGTTTGTTTGCCGGATCA |
| | AGAGCTACCAACTCTTTTTCCGAAGGTAACTGGCTTCAGCAGAGCGCAGATACCAA |
| | ATACTGTCCTTCTAGTGTAGCCGTAGTTAGGCCACCACTTCAAGAACTCTGTAGCA |
| | CCGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAGC |
| | GGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCG |
| | CACGAGGGAGCTTCCAGGGGGAAACGCCTGGTATCTTTATAGTCCTGTCGGGTTT |
| | CGCCACCTCTGACTTGAGCGTCGATTTTTGTGATGCTCGTCAGGGGGGCGGAGCC |
| | TATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTACC |
| | GCCTTTGAGTGAGCTGATACCGCTCGCCGCAGCCGAACGACCGAGCGCAGCGAG |
| | TCAGTGAGCGAGGAAGCGGAAGAGCGCCTGATGCGGTATTTTCTCCTTACGCATCT |
| | GTGCGGTATTTCACACCGCATATATGGTGCACTCTCAGTACAATCTGCTCTGATGC |
| | CGCATAGTTAAGCCAGTATACACTCCGCTATCGCTACGTGACTGGGTCATGGCTGC |
| | GCCCCGACACCCGCCAACACCCGCTGACGCGCCCTGACGGGCTTGTCTGCTCCC |
| | GGCATCCGCTTACAGACAAGCTGTGACCGTCTCCGGGAGCTGCATGTGTCAGAGG |
| | TTTTCACCGTCATCACCGAAACGCGCGAGGCAGCTGCGGTAAAGCTCATCAGCGT |
| | GGTCGTGAAGCGATTCACAGATGTCTGCCTGTTCATCCGCGTCCAGCTCGTTGAGT |
| | TTCTCCAGAAGCGTTAATGTCTGGCTTCTGATAAAGCGGGCCATGTTAAGGGCGGT |
| | TTTTTCCTGTTTGGTCACTGATGCCTCCGTGTAAGGGGGATTTCTGTTCATGGGGG |
| | TAATGATACCGATGAAACGAGAGAGGATGCTCACGATACGGGTTACTGATGATGAA |
| | CATGCCCGGTTACTGGAACGTTGTGAGGGTAAACAACTGGCGGTATGGATGCGGC |
| | GGGACCAGAGAAAAATCACTCAGGGTCAATGCCAGCGCTTCGTTAATACAGATGTA |
| | GGTGTTCCACAGGGTAGCCAGCAGCATCCTGCGATGCAGATCCGGAACATAATGG |
| | TGCAGGGCGCTGACTTCCGCGTTTCCAGACTTTACGAAACACGGAAACCGAAGAC |
| | CATTCATGTTGTTGCTCAGGTCGCAGACGTTTTGCAGCAGCAGTCGCTTCACGTTC |
| | GCTCGCGTATCGGTGATTCATTCTGCTAACCAGTAAGGCAACCCCGCCAGCCTAGC |
| | CGGGTCCTCAACGACAGGAGCACGATCATGCGCACCCGTGGGGCCGCCATGCCG |
| | GCGATAATGGCCTGCTTCTCGCCGAAACGTTTGGTGGCGGGACCAGTGACGAAGG |
| | CTTGAGCGAGGGCGTGCAAGATTCCGAATACCGCAAGCGACAGGCCGATCATCGT |
| | CGCGCTCCAGCGAAAGCGGTCCTCGCCGAAAATGACCCAGAGCGCTGCCGGCAC |
| | CTGTCCTACGAGTTGCATGATAAAGAAGACAGTCATAAGTGCGGCGACGATAGTCA |
| | TGCCCCGCGCCCACCGGAAGGAGCTGACTGGGTTGAAGGCTCTCAAGGGCATCG |
| | GTCGAGATCCCGGTGCCTAATGAGTGAGCTAACTTACATTAATTGCGTTGCGCTCA |
| | CTGCCCGCTTTCCAGTCGGGAAACCTGTCGTGCCAGCTGCATTAATGAATCGGCCA |
| | ACGCGCGGGGAGAGGCGGTTTGCGTATTGGGCGCCAGGGTGGTTTTTCTTTTCAC |
| | CAGTGAGACGGGCAACAGCTGATTGCCCTTCACCGCCTGGCCCTGAGAGAGTTGC |
| | AGCAAGCGGTCCACGCTGGTTTGCCCCAGCAGGCGAAAATCCTGTTTGATGGTGG |
| | TTAACGGCGGGATATAACATGAGCTGTCTTCGGTATCGTCGTATCCCACTACCGAG |
| | ATATCCGCACCAACGCGCAGCCCGGACTCGGTAATGGCGCGCATTGCGCCCAGC |
| | GCCATCTGATCGTTGGCAACCAGCATCGCAGTGGGAACGATGCCCTCATTCAGCAT |
| | TTGCATGGTTTGTTGAAAACCGGACATGGCACTCCAGTCGCCTTCCCGTTCCGCTA |
| | TCGGCTGAATTTGATTGCGAGTGAGATATTTATGCCAGCCAGCCAGACGCAGACGC |
| | GCCGAGACAGAACTTAATGGGCCCGCTAACAGCGCGATTTGCTGGTGACCCAATG |
| | CGACCAGATGCTCCACGCCCAGTCGCGTACCGTCTTCATGGGAGAAAATAATACTG |
| | TTGATGGGTGTCTGGTCAGAGACATCAAGAAATAACGCCGGAACATTAGTGCAGGC |
| | AGCTTCCACAGCAATGGCATCCTGGTCATCCAGCGGATAGTTAATGATCAGCCCAC |
| | TGACGCGTTGCGCGAGAAGATTGTGCACCGCCGCTTTACAGGCTTCGACGCCGCT |
| | TCGTTCTACCATCGACACCACCACGCTGGCACCCAGTTGATCGGCGCGAGATTTAA |
| | TCGCCGCGACAATTTGCGACGGCGCGTGCAGGGCCAGACTGGAGGTGGCAACGC |
| | CAATCAGCAACGACTGTTTGCCCGCCAGTTGTTGTGCCACGCGGTTGGGAATGTAA |
| | TTCAGCTCCGCCATCGCCGCTTCCACTTTTTCCCGCGTTTTCGCAGAAACGTGGCT |
| | GGCCTGGTTCACCACGCGGGAAACGGTCTGATAAGAGACACCGGCATACTCTGCG |
| | ACATCGTATAACGTTACTGGTTTCACATTCACCACCCTGAATTGACTCTCTTCCGGG |
| | CGCTATCATGCCATACCGCGAAAGGTTTTGCGCCATTCGATGGTGTCCGGGATCTC |
| | GACGCTCTCCCTTATGCGACTCCTGCATTAGGAAGCAGCCCAGTAGTAGGTTGAG |
| | GCCGTTGAGCACCGCCGCCGCAAGGAATGGTGCATGCAAGGAGATGGCGCCCAA |
| | CAGTCCCCCGGCCACGGGGCCTGCCACCATACCCACGCCGAAACAAGCGCTCAT |
| | GAGCCCGAAGTGGCGAGCCCGATCTTCCCCATCGGTGATGTCGGCGATATAGGCG |
| | CCAGCAACCGCACCTGTGGCGCCGGTGATGCCGGCCACGATGCGTCCGGCGTAG |
| | AGGATCGAGATCTGAAATTAATACGACTCACTATAGGGGAATTGTGAGCGGATAAC |
| | AATTCCCCTCTAGAAATAATTTTGTTTAACTTTAAGAAGGAGATATACCATGAAACAT |
| | CACCATCACCATCACATGTCCCCTATACTAGGTTATTGGAAAATTAAGGGCCTTGTG |
| | CAACCCACTCGACTTCTTTTGGAATATCTTGAAGAAAAATATGAAGAGCATTTGTAT |
| | GAGCGCGATGAAGGTGATAAATGGCGAAACAAAAAGTTTGAATTGGGTTTGGAGTT |
| | TCCCAATCTTCCTTATTATATTGATGGTGATGTTAAATTAACACAGTCTATGGCCATC |
| | ATACGTTATATAGCTGACAAGCACAACATGTTGGGTGGTTGTCCAAAAGAGCGTGC |
| | AGAGATTTCAATGCTTGAAGGAGCGGTTTTGGATATTAGATACGGTGTTTCGAGAAT |
| | TGCATATAGTAAAGACTTTGAAACTCTCAAAGTTGATTTTCTTAGCAAGCTACCTGA |
| | AATGCTGAAAATGTTCGAAGATCGTTTATGTCATAAAACATATTTAAATGGTGATCAT |
| | GTAACCCATCCTGACTTCATGTTGTATGACGCTCTTGATGTTGTTTTATACATGGAC |
| | CCAATGTGCCTGGATGCGTTCCCAAAATTAGTTTGTTTTAAAAAACGTATTGAAGCT |
| | ATCCCACAAATTGATAAGTACTTGAAATCCAGCAAGTATATAGCATGGCCTTTGCAG |
| | GGCTGGCAAGCCACGTTTGGTGGTGGCGACCATCCTCCAAAACCCATGAGCGATT |
| | ACGACATCCCCACTACTGAGAATCTTTATTTTCAGGGCGCCATGGACGTTGTAAGC |
| | TTAGATAAACCGTTCATGTATTTTGAGGAAATCGATAACGAACTGGATTATGAACCG |
| | GAATCGGCGAATGAGGTGGCTAAAAAATTGCCATACCAGGGTCAACTTAAATTATT |
| | ATTGGGGGAACTGTTTTTTTTAAGTAAGCTTCAGCGTCATGGGATTCTTGACGGTG |
| | CAACGGTGGTATACATTGGGTCGGCCCCTGGCACTCATATCCGCTACTTACGCGAC |
| | CACTTCTACAACTTAGGCGTCATCATCAAATGGATGTTGATCGATGGGCGCCATCA |
| | CGACCCCATTCTTAATGGCTTGCGCGATGTAACACTTGTTACACGTTTCGTCGATG |
| | AAGAGTACTTACGTAGCATTAAAAAACAACTTCACCCATCCAAAATCATTCTGATCT |
| | CTGACGTACGTAGCAAACGTGGGGGTAATGAACCAAGTACAGCTGACCTTCTGTCT |
| | AACTATGCTTTACAAAACGTGATGATTTCCATCTTGAATCCCGTTGCTTCATCATTGA |
| | AGTGGCGTTGCCCCTTCCCGGACCAGTGGATCAAAGATTTCTACATCCCTCACGGC |
| | AACAAGATGTTGCAACCGTTCGCGCCATCCTATTCGGCCGAAATGCGCTTGTTGTC |
| | CATCTACACAGGCGAGAACATGCGTTTAACTCGTGTAACAAAGTCGGATGCTGTAA |
| | ACTACGAAAAAAAGATGTACTATCTTAATAAGATTGTTCGTAACAAAGTGGTTGTGA |
| | ATTTCGATTACCCTAACCAGGAGTACGATTATTTTCATATGTATTTTATGTTGCGTAC |
| | CGTTTATTGTAATAAGACTTTTCCGACAACTAAGGCAAAGGTGCTGTTTCTGCAGCA |
| | ATCGATTTTCCGCTTCTTAAACATCCCCACCACAAGTACAGAAAAAGTCTCACACGA |
| | GCCCATCCAACGCAAGATTTCTTCTAAAAATAGCATGAGCAAAAATCGCAATTCAAA |
| | ACGCTCGGTTCGTAGTAACAAATAGCTCGAGCACCACCACCACCACCACTGAGATC |
| | CGGCTGCTAACAAAGCCCGAAAGGAAGCTGAGTTGGCTGCTGCCACCGCTGAGCA |
| | ATAACTAGCATAACCCCTTGGGGCCTCTAAACGGGTCTTGAGGGGTTTTTTGCTGA |
| | AAGGAGGAACTATATCCGGATTCCGGAT |
| SEQ ID NO: | ATGGACGCTAATGTCGTCTCGTCAAGTACGATCGCTACCTACATTGACGCCTTGGC |
| 27 | TAGAGCTTGTATTTATCAAACCACCGTTGATTACTCTGACGAACGTAGTTAACATTT |
| D1 | GAAAAATGCTAGCGAGCTTGAACAGCGCAGCACTGCTTATGAGATCAATAATGAGT |
| sequence | CAACCATTCAAGAGTCCTTTATTCGTTTTACCGTCACGAACAAAGAGGGCGTTAAAA |
| codon | TGGTAGACGCCATTGATAACATCGTATGGGAGAAGAAATCACTGGTTACTGAAAAC |
| optimized | TCCGCACCAAGATCCCTCTTAGTAAAGTGCATGGATTAGATGTCAAAAATGTTCAGC |
| according to | CGCCTGCACAAAGAATGTCTGTTGCGTCTGTCTACCGAGGAACGTCATATTTTCCT |
| method A | TGACTACAAGAAATACGGTTCGAGTATTCGCTTAGAACTTGTGAACCTGATCCAGG |
| | CCAAGACTAAAAATTTTACAATTGATTTTAAATTAAAATACTTCTTGGGAAGTGGGGC |
| | ACAATCCAAGTCGTCCTTGTTACATGCTATCAACCATOCCAAATCCCGTCCTAACAC |
| | AAGCTTGGAGATTGAATTCACACCGCGTGATAACGAAACTGTTCCTTATGACGAGC |
| | TGATTAAAGAATTGACGACGCTTTCTCGCCATATCTTCATGGCCTCTCCCGAAAACG |
| | TCATCCTTAGCCCTCCAATCAATGCACCAATTAAGACATTTATGTTACCGAAGCAAG |
| | ACATTGTAGGCTTGGATCTTGAAAATCTTTATGCTGTTACCAAAACAGATGGTATTC |
| | CAATTACAATTCGTGTTACATCTAACGGCCTTTACTGCTATTTCACCCACTTGGGAT |
| | ATATCATCCGTTATCCGGTTAAGCGTATTATCGACTCTGAGGTAGTAGTTTTTGGCG |
| | AAGCAGTAAAGGACAAGAATTGGACAGTATACCTGATTAAATTAATTGAACCGGTAA |
| | ATGCCATTAATGATCGCTTAGAGGAAAGCAAGTATGTCGAATCTAAACTTGTTGATA |
| | TCTGTGATCGCATCGTATTTAAAAGTAAAAAATACGAAGGGCCTTTTACTACAACAA |
| | GTGAAGTTGTTGACATGTTATCTACCTATTTACCAAAACAACCTGAAGGTGTTATTTT |
| | GTTCTATTCCAAGGGCCCCAAATCGAACATTGACTTTAAGATCAAGAAGGAAAACA |
| | CTATTGATCAGACCGCCAACGTAGTATTCCGCTATATGTCCAGTGAACCGATTATTT |
| | TCGGCGAGAGTAGCATTTTCGTTGAATATAAAAAATTTTCCAACGACAAGGGCTTCC |
| | CGAAAGAGTATGGGTCAGGCAAGATTGTATTGTATAATGGTGTCAATTATOTTAATA |
| | ACATCTATTGCCTTGAGTATATCAATACGCACAACGAGGTGGGCATTAAGTCCGTA |
| | GTAGTGCCCATCAAGTTCATCGCTGAGTTCTTGGTGAACGGGGAGATCTTAAAACC |
| | TCGTATCGACAAGACTATGAAATATATCAACTCCGAAGACTATTATGGGAATCAGCA |
| | CAACATCATTGTAGAGCACCTGCGCGACCAGTCCATTAAGATTGGAGATATTTTTAA |
| | CGAAGATAAGCTGTCCGACGTCGGGCACCAGTACGCGAACAACGATAAGTTCCGT |
| | TTGAATCCTGAAGTATCATATTTCACAAACAAACGCACACGTGGTCCACTGGGGAT |
| | CTTGTCGAACTATGTCAAAACGCTGTTAATCTCTATGTATTGTTCAAAGACTTTCCTT |
| | GATGATAGCAACAAGCGTAAGGTACTGGCTATCGATTTTGGAAACGGCGCCGACTT |
| | GGAGAAATACTTTTACGGCGAAATCGCTCTGTTAGTTGCTACAGATCCAGACGCAG |
| | ATGCGATCGCTCGCGGTAACGAGCGCTATAATAAACTTAACTCCGGCATTAAAACT |
| | AAGTATTACAAGTTCGACTACATCCAAGAGACTATTCGTAGTGATACGTTCGTGAGT |
| | TCGGTGCGCGAGGTATTCTATTTCGGTAAATTTAATATCATCGACTGGCAGTTTGCC |
| | ATCCATTATAGTTTCCATCCACGCCACTATGCCACAGTTATGAATAACTTAAGTGAG |
| | CTTACAGCAAGCGGTGGTAAAGTGCTGATTACCACAATGGACGGCGATAAGTTGTC |
| | TAAGTTGACGGACAAAAAAACATTCATTATCCATAAGAATCTGCCTTCAAGTGAAAA |
| | TTATATGTCAGTAGAGAAGATCGCCGACGACCGCATCGTAGTTTACAATCCGAGTA |
| | CCATGAGTACTCCCATGACGGAGTACATCATCAAAAAGAATGACATCGTCCGTGTT |
| | TTCAATGAGTACGGCTTCGTACTTGTAGATAACGTCGATTTTGCAACGATTATTGAA |
| | CGCTCAAAGAAGTTTATTAATGGGGCGTCAACAATGGAGGATCGCCCTTCCACTCG |
| | CAATTTCTTTGAATTAAATCGTGGGGCCATCAAGTGCGAGGGCCTGGATGTAGAAG |
| | ATTTATTGTCATATTACGTCGTGTACGTTTTCTCCAAGCGTTAA |
| SEQ ID NO: | ATGGACGAGATTGTAAAGAACATTCGTGAAGGAACGCACGTGTTGCTTCCGTTCTA |
| 28 | CGAGACCTTACCAGAGTTGAACTTATCGCTTGGGAAATCCCCGTTGCCTTCGCTTG |
| D12 | AGTATGGTGCAAACTATTTCCTGCAAATTAGTCGCGTAAATGATCTTAACCGTATGC |
| sequence | CGACGGACATGCTGAAATTATTTACCCACGATATTATGTTACCCGAATCAGATCTGG |
| codon | ACAAAGTATATGAGATTCTTAAAATCAACTCTGTGAAATACTACGGCCGTAGCACGA |
| optimized | AAGCCGACGCGGTCGTCGCAGACCTTTCTGCGCGCAACAAGCTTTTCAAGCGCGA |
| according to | GCGCGACGCGATTAAATCTAACAACCATTTGACCGAAAACAACCTTTACATCAGCG |
| method A | ACTATAAGATGCTGACCTTTGACGTATTCCGCCCGTTGTTCGACTTCGTGAATGAAA |
| | AATATTGCATTATTAAATTACCAACGTTGTTTGGCCGTGGCGTAATCGACACCATGC |
| | GTATTTACTGTTCCTTGTTTAAAAATGTTCGCTTGCTTAAATGCGTTTCTGATAGCTG |
| | GTTGAAGGATTCTGCGATCATGGTCGCTTCCGATGTATGTAAAAAAAACTTGGACTT |
| | GTTCATGTCGCATGTTAAATCGGTTACTAAATCTAGCTCATGGAAGGATGTCAATTC |
| | CGTTCAGTTCTCAATTCTTAACAACCCCGTAGATACGGAGTTCATCAACAAATTTTT |
| | AGAGTTCTCCAACCGCGTGTATGAGGCACTGTACTACGTTCATTCTTTATTATACTC |
| | TAGCATGACCAGTGATTCAAAAAGCATTGAAAACAAACATCAGCGCCGCCTGGTTA |
| | AGCTTCTGTTATAA |
| SEQ ID NO: | ATGCCGTCCGTTCAAGAAGTCGAAAAACTTCTGCATGTTCTGGATCGTAACGGCGA |
| 29, | TGGAAAAGTATCCGCCGAGGAATTGAAGGCATTTGCTGACGATTCGAAATGTCCGT |
| Fh8 | TGGATAGTAATAAGATCAAAGCATTCATCAAAGAGCATGACAAGAACAAGGACGGG |
| | AAATTGGATTTGAAGGAGTTGGTGTCTATTCTTAGCTCT |
| SEQ ID NO: | ATGGACGTTGTAAGCTTAGATAAACCGTTCATGTATTTTGAGGAAATCGATAACGAA |
| 30, | CTGGATTATGAACCGGAATCGGCGAATGAGGTGGCTAAAAAATTGCCATACCAGGG |
| VP39 | TCAACTTAAATTATTATTGGGGGAACTGTTTTTTTTAAGTAAGCTTCAGCGTCATGG |
| sequence | GATTCTTGACGGTGCAACGGTGGTATACATTGGGTCGGCCCCTGGCACTCATATCC |
| codon | GCTACTTACGCGACCACTTCTACAACTTAGGCGTCATCATCAAATGGATGTTGATC |
| optimized | GATGGGCGCCATCACGACCCCATTCTTAATGGCTTGCGCGATGTAACACTTGTTAC |
| according to | ACGTTTCGTCGATGAAGAGTACTTACGTAGCATTAAAAAACAACTTCACCCATCCAA |
| method A | AATCATTCTGATCTCTGACGTACGTAGCAAACGTGGGGGTAATGAACCAAGTACAG |
| | CTGACCTTCTGTCTAACTATGCTTTACAAAACGTGATGATTTCCATCTTGAATCCCG |
| | TTGCTTCATCATTGAAGTGGCGTTGCCCCTTCCCGGACCAGTGGATCAAAGATTTC |
| | TACATCCCTCACGGCAACAAGATGTTGCAACCGTTCGCGCCATCCTATTCGGCCGA |
| | AATGCGCTTGTTGTCCATCTACACAGGCGAGAACATGCGTTTAACTCGTGTAACAA |
| | AGTCGGATGCTGTAAACTACGAAAAAAAGATGTACTATCTTAATAAGATTGTTCGTA |
| | ACAAAGTGGTTGTGAATTTCGATTACCCTAACCAGGAGTACGATTATTTTCATATGT |
| | ATTTTATGTTGCGTACCGTTTATTGTAATAAGACTTTTCCGACAACTAAGGCAAAGG |
| | TGCTGTTTCTGCAGCAATCGATTTTCCGCTTCTTAAACATCCCCACCACAAGTACAG |
| | AAAAAGTCTCACACGAGCCCATCCAACGCAAGATTTCTTCTAAAAATAGCATGAGCA |
| | AAAATCGCAATTCAAAACGCTCGGTTCGTAGTAACAAATAG |
| SEQ ID NO: | ATGCCTGAACCCCACGCCGTCTTATACGTAACTAACGAGTTATCTCATATTGTAAAA |
| 31 | AACGGGTTTCTTCCCATTTGGAAACTTACCGGCGATGAGTCTTTGAATGACCTGTG |
| VP4-His6 | GCTTGAAAATGGAAAATACGCCACCGACGTATACGCATACGGAGATGTATCTAAAT |
| sequence | GGACGATCCGCCAGTTGCGCGGACACGGGTTCATTTTCATTAGCACGCATAAAAAC |
| codon | GTGCAGCTGGCAGACATTATTAAGACTGTGGACGTCCGTATCCCTCGCGAGGTAG |
| optimized | CACGCTCGCATGATATGAAAGCATTCGAAAATGAAATCTCACGTCGCCGTATCCGC |
| according to | ATGCGCAAAGGATTCGGTGACGCGCTGCGTAACTATGCGTTTAAAATGGCGATTGA |
| method B | GTTTCATGGCAGCGAAGCCGAAACGTTGAACGACGCCAACCCACGTCTTCACAAG |
| | ATTTACGGGATGCCCGAAATCCCTCCCTTGTACATGGAGTACGCCGAGATCGGTAC |
| | TCGTTTCGATGACGAACCCACAGATGAAAAGTTGGTTAGTATGCTTGACTACATTGT |
| | TTATTCCGCGGAGGAGGTTCATTACGTGGGCTGCGGAGACTTACGCACACTTATGC |
| | AGTTCAAGAAGCGCTCTCCTGGCCGCTTTCGCCGCGTATTGTGGCATGTGTACGAC |
| | CCCATCGCGCCCGAATGTAGCGATCCTAATGTAATTGTACATAACATCATGGTCGA |
| | TTCCAAAAAAGATATTCTTAAACATATGAACTTCTTAAAACGTGTGGAGCGCCTTTTC |
| | ATTTGGGATGTCAGCTCGGATCGCTCTCAGATGAACGATCACGAGTGGGAAACGA |
| | CGCGTTTCGCCGAGGACCGTTTAGGTGAGGAAATCGCGTACGAGATGGGGGGGG |
| | CTTTCTCATCGGCTTTGATTAAGCACCGCATCCCAAACAGCAAGGACGAATATCAC |
| | TGTATTTCGACCTACCTTTTCCCCCAACCGGGAGCAGACGCAGACATGTACGAGCT |
| | GCGCAACTTCATGCGCTTACGTGGGTATTCACATGTCGACCGTCATATGCACCCAG |
| | ACGCTTCAGTGACTAAAGTAGTGAGTCGTGACGTTCGCAAGATGGTGGAATTGTAC |
| | CACGGCCGTGACCGTGGTCGTTTCTTAAAGAAACGCTTATTTGAACATCTGCACAT |
| | CGTCCGTAAGAATGGGCTGCTTCACGAATCGGACGAGCCACGTGCGGACTTATTC |
| | TATTTGACAAACCGCTGCAACATGGGTTTGGAGCCCAGTATCTATGAAGTGATGAA |
| | GAAATCCGTCATTGCCACCGCATGGGTGGGTCGTGCGCCTCTTTATGACTATGACG |
| | ATTTTGCATTGCCGCGTTCAACCGTTATGTTAAACGGTAGTTACCGCGATATCCGTA |
| | TTTTGGACGGGAACGGCGCGATCTTATTTCTTATGTGGCGCTATCCCGATATTGTC |
| | AAGAAGGACCTGACATATGACCCCGCGTGGGCCATGAATTTTGCGGTCAGCTTAAA |
| | AGAGCCTATCCCGGATCCACCTGTTCCAGACATCAGTTTATGCCGCTTTATCGGGT |
| | TACGCGTCGAATCGAGCGTTCTTCGCGTTCGTAATCCAACGTTGCATGAGACGGCT |
| | GACGAGTTGAAACGTATGGGCTTAGACCTTTCTGGCCATCTGTACGTCACCTTGAT |
| | GAGCGGGGCATATGTGACCGACCTTTTTTGGTGGTTTAAGATGATTTTAGATTGGT |
| | CTGCGCAAAACAAGGAGCAGAAGCTGCGTGACCTTAAGCGCTCAGCGGCGGAGGT |
| | AATCGAGTGGAAAGAACAGATGGCCGAACGTCCGTGGCACGTCCGTAACGACCTT |
| | ATCCGCGCTCTGCGCGAATACAAACGCAAGATGGGCATGCGTGAAGGTGCATCGA |
| | TCGACTCGTGGCTGGAATTGCTTCGCCACCTTATGAAACACCACCATCACCATCAC |
| SEQ ID NO: | ATGCCTGAACCCCACGCCGTCTTATACGTAACTAACGAGTTATCTCATATTGTAAAA |
| 32 | AACGGGTTTCTTCCCATTTGGAAACTTACCGGCGATGAGTCTTTGAATGACCTGTG |
| VP4-noTEV- | GCTTGAAAATGGAAAATACGCCACCGACGTATACGCATACGGAGATGTATCTAAAT |
| Fh8-His6 | GGACGATCCGCCAGTTGCGCGGACACGGGTTCATTTTCATTAGCACGCATAAAAAC |
| sequence | GTGCAGCTGGCAGACATTATTAAGACTGTGGACGTCCGTATCCCTCGCGAGGTAG |
| codon | CACGCTCGCATGATATGAAAGCATTCGAAAATGAAATCTCACGTCGCCGTATCCGC |
| optimized | ATGCGCAAAGGATTCGGTGACGCGCTGCGTAACTATGCGTTTAAAATGGCGATTGA |
| according to | GTTTCATGGCAGCGAAGCCGAAACGTTGAACGACGCCAACCCACGTCTTCACAAG |
| method B | ATTTACGGGATGCCCGAAATCCCTCCCTTGTACATGGAGTACGCCGAGATCGGTAC |
| | TCGTTTCGATGACGAACCCACAGATGAAAAGTTGGTTAGTATGCTTGACTACATTGT |
| | TTATTCCGCGGAGGAGGTTCATTACGTGGGCTGCGGAGACTTACGCACACTTATGC |
| | AGTTCAAGAAGCGCTCTCCTGGCCGCTTTCGCCGCGTATTGTGGCATGTGTACGAC |
| | CCCATCGCGCCCGAATGTAGCGATCCTAATGTAATTGTACATAACATCATGGTCGA |
| | TTCCAAAAAAGATATTCTTAAACATATGAACTTCTTAAAACGTGTGGAGCGCCTTTTC |
| | ATTTGGGATGTCAGCTCGGATCGCTCTCAGATGAACGATCACGAGTGGGAAACGA |
| | CGCGTTTCGCCGAGGACCGTTTAGGTGAGGAAATCGCGTACGAGATGGGGGGGG |
| | CTTTCTCATCGGCTTTGATTAAGCACCGCATCCCAAACAGCAAGGACGAATATCAC |
| | TGTATTTCGACCTACCTTTTCCCCCAACCGGGAGCAGACGCAGACATGTACGAGCT |
| | GCGCAACTTCATGCGCTTACGTGGGTATTCACATGTCGACCGTCATATGCACCCAG |
| | ACGCTTCAGTGACTAAAGTAGTGAGTCGTGACGTTCGCAAGATGGTGGAATTGTAC |
| | CACGGCCGTGACCGTGGTCGTTTCTTAAAGAAACGCTTATTTGAACATCTGCACAT |
| | CGTCCGTAAGAATGGGCTGCTTCACGAATCGGACGAGCCACGTGCGGACTTATTC |
| | TATTTGACAAACCGCTGCAACATGGGTTTGGAGCCCAGTATCTATGAAGTGATGAA |
| | GAAATCCGTCATTGCCACCGCATGGGTGGGTCGTGCGCCTCTTTATGACTATGACG |
| | ATTTTGCATTGCCGCGTTCAACCGTTATGTTAAACGGTAGTTACCGCGATATCCGTA |
| | TTTTGGACGGGAACGGCGCGATCTTATTTCTTATGTGGCGCTATCCCGATATTGTC |
| | AAGAAGGACCTGACATATGACCCCGCGTGGGCCATGAATTTTGCGGTCAGCTTAAA |
| | AGAGCCTATCCCGGATCCACCTGTTCCAGACATCAGTTTATGCCGCTTTATCGGGT |
| | TACGCGTCGAATCGAGCGTTCTTCGCGTTCGTAATCCAACGTTGCATGAGACGGCT |
| | GACGAGTTGAAACGTATGGGCTTAGACCTTTCTGGCCATCTGTACGTCACCTTGAT |
| | GAGCGGGGCATATGTGACCGACCTTTTTTGGTGGTTTAAGATGATTTTAGATTGGT |
| | CTGCGCAAAACAAGGAGCAGAAGCTGCGTGACCTTAAGCGCTCAGCGGCGGAGGT |
| | AATCGAGTGGAAAGAACAGATGGCCGAACGTCCGTGGCACGTCCGTAACGACCTT |
| | ATCCGCGCTCTGCGCGAATACAAACGCAAGATGGGCATGCGTGAAGGTGCATCGA |
| | TCGACTCGTGGCTGGAATTGCTTCGCCACCTTATGAGCGATTACGACATCGACACT |
| | ACTATGCCCTCCGTCCAGGAGGTCGAAAAATTATTACATGTGTTAGACCGTAACGG |
| | AGATGGAAAAGTTAGCGCCGAGGAGCTGAAGGCATTCGCGGACGACTCCAAGTGC |
| | CCTCTTGACTCGAACAAGATTAAAGCATTTATTAAAGAACACGATAAGAATAAAGAC |
| | GGGAAGTTGGACTTGAAGGAATTGGTATCGATCTTATCGAGTATGAAACACCACCA |
| | TCACCATCAC |
| SEQ ID NO: | ATGCCTGAACCCCACGCCGTCTTATACGTAACTAACGAGTTATCTCATATTGTAAAA |
| 33 | AACGGGTTTCTTCCCATTTGGAAACTTACCGGCGATGAGTCTTTGAATGACCTGTG |
| VP4-TEV- | GCTTGAAAATGGAAAATACGCCACCGACGTATACGCATACGGAGATGTATCTAAAT |
| Fh8-His6 | GGACGATCCGCCAGTTGCGCGGACACGGGTTCATTTTCATTAGCACGCATAAAAAC |
| sequence | GTGCAGCTGGCAGACATTATTAAGACTGTGGACGTCCGTATCCCTCGCGAGGTAG |
| codon | CACGCTCGCATGATATGAAAGCATTCGAAAATGAAATCTCACGTCGCCGTATCCGC |
| optimized | ATGCGCAAAGGATTCGGTGACGCGCTGCGTAACTATGCGTTTAAAATGGCGATTGA |
| according to | GTTTCATGGCAGCGAAGCCGAAACGTTGAACGACGCCAACCCACGTCTTCACAAG |
| method B | ATTTACGGGATGCCCGAAATCCCTCCCTTGTACATGGAGTACGCCGAGATCGGTAC |
| | TCGTTTCGATGACGAACCCACAGATGAAAAGTTGGTTAGTATGCTTGACTACATTGT |
| | TTATTCCGCGGAGGAGGTTCATTACGTGGGCTGCGGAGACTTACGCACACTTATGC |
| | AGTTCAAGAAGCGCTCTCCTGGCCGCTTTCGCCGCGTATTGTGGCATGTGTACGAC |
| | CCCATCGCGCCCGAATGTAGCGATCCTAATGTAATTGTACATAACATCATGGTCGA |
| | TTCCAAAAAAGATATTCTTAAACATATGAACTTCTTAAAACGTGTGGAGCGCCTTTTC |
| | ATTTGGGATGTCAGCTCGGATCGCTCTCAGATGAACGATCACGAGTGGGAAACGA |
| | CGCGTTTCGCCGAGGACCGTTTAGGTGAGGAAATCGCGTACGAGATGGGGGGGG |
| | CTTTCTCATCGGCTTTGATTAAGCACCGCATCCCAAACAGCAAGGACGAATATCAC |
| | TGTATTTCGACCTACCTTTTCCCCCAACCGGGAGCAGACGCAGACATGTACGAGCT |
| | GCGCAACTTCATGCGCTTACGTGGGTATTCACATGTCGACCGTCATATGCACCCAG |
| | ACGCTTCAGTGACTAAAGTAGTGAGTCGTGACGTTCGCAAGATGGTGGAATTGTAC |
| | CACGGCCGTGACCGTGGTCGTTTCTTAAAGAAACGCTTATTTGAACATCTGCACAT |
| | CGTCCGTAAGAATGGGCTGCTTCACGAATCGGACGAGCCACGTGCGGACTTATTC |
| | TATTTGACAAACCGCTGCAACATGGGTTTGGAGCCCAGTATCTATGAAGTGATGAA |
| | GAAATCCGTCATTGCCACCGCATGGGGGGTCGTGCGCCTCTTTATGACTATGACG |
| | ATTTTGCATTGCCGCGTTCAACCGTTATGTTAAACGGTAGTTACCGCGATATCCGTA |
| | TTTTGGACGGGAACGGCGCGATCTTATTTCTTATGTGGCGCTATCCCGATATTGTC |
| | AAGAAGGACCTGACATATGACCCCGCGTGGGCCATGAATTTTGCGGTCAGCTTAAA |
| | AGAGCCTATCCCGGATCCACCTGTTCCAGACATCAGTTTATGCCGCTTTATCGGGT |
| | TACGCGTCGAATCGAGCGTTCTTCGCGTTCGTAATCCAACGTTGCATGAGACGGCT |
| | GACGAGTTGAAACGTATGGGCTTAGACCTTTCTGGCCATCTGTACGTCACCTTGAT |
| | GAGCGGGGCATATGTGACCGACCTTTTTTGGTGGTTTAAGATGATTTTAGATTGGT |
| | CTGCGCAAAACAAGGAGCAGAAGCTGCGTGACCTTAAGCGCTCAGCGGCGGAGGT |
| | AATCGAGTGGAAAGAACAGATGGCCGAACGTCCGTGGCACGTCCGTAACGACCTT |
| | ATCCGCGCTCTGCGCGAATACAAACGCAAGATGGGCATGCGTGAAGGTGCATCGA |
| | TCGACTCGTGGCTGGAATTGCTTCGCCACCTTCCCATGAGCGATTACGACATCCCC |
| | ACTACTGAGAATCTTTATTTTCAGGGCGCCATGCCCTCCGTCCAGGAGGTCGAAAA |
| | ATTATTACATGTGTTAGACCGTAACGGAGATGGAAAAGTTAGCGCCGAGGAGCTGA |
| | AGGCATTCGCGGACGACTCCAAGTGCCCTCTTGACTCGAACAAGATTAAAGCATTT |
| | ATTAAAGAACACGATAAGAATAAAGACGGGAAGTTGGACTTGAAGGAATTGGTATC |
| | GATCTTATCGAGTATGAAACACCACCATCACCATCAC |
| SEQ ID NO: | ATGAAACATCACCATCACCATCACATGAAAATCGAAGAAGGTAAACTGGTAATCTG |
| 34 | GATTAACGGCGATAAAGGCTATAACGGTCTCGCTGAAGTCGGTAAGAAATTCGAGA |
| His6-MBP- | AAGATACCGGAATTAAAGTCACCGTTGAGCATCCGGATAAACTGGAAGAGAAATTC |
| VP4 | CCACAGGTTGCGGCAACTGGCGATGGCCCTGACATTATCTTCTGGGCACACGACC |
| sequence | GCTTTGGTGGCTACGCTCAATCTGGCCTGTTGGCTGAAATCACCCCGGACAAAGC |
| codon | GTTCCAGGACAAGCTGTATCCGTTTACCTGGGATGCCGTACGTTACAACGGCAAGC |
| optimized | TGATTGCTTACCCGATCGCTGTTGAAGCGTTATCGCTGATTTATAACAAAGATCTGC |
| according to | TGCCGAACCCGCCAAAAACCTGGGAAGAGATCCCGGCGCTGGATAAAGAACTGAA |
| method A | AGCGAAAGGTAAGAGCGCGCTGATGTTCAACCTGCAAGAACCGTACTTCACCTGG |
| | CCGCTGATTGCTGCTGACGGGGGTTATGCGTTCAAGTATGAAAACGGCAAGTACG |
| | ACATTAAAGACGTGGGCGTGGATAACGCTGGCGCGAAAGCGGGTCTGACCTTCCT |
| | GGTTGACCTGATTAAAAACAAACACATGAATGCAGACACCGATTACTCCATCGCAG |
| | AAGCTGCCTTTAATAAAGGCGAAACAGCGATGACCATCAACGGCCCGTGGGCATG |
| | GTCCAACATCGACACCAGCAAAGTGAATTATGGTGTAACGGTACTGCCGACCTTCA |
| | AGGGTCAACCATCCAAACCGTTCGTTGGCGTGCTGAGCGCAGGTATTAACGCCGC |
| | CAGTCCGAACAAAGAGCTGGCAAAAGAGTTCCTCGAAAACTATCTGCTGACTGATG |
| | AAGGTCTGGAAGCGGTTAATAAAGACAAACCGCTGGGTGCCGTAGCGCTGAAGTC |
| | TTACGAGGAAGAGTTGGCGAAAGATCCACGTATTGCCGCCACTATGGAAAACGCC |
| | CAGAAAGGTGAAATCATGCCGAACATCCCGCAGATGTCCGCTTTCTGGTATGCCGT |
| | GCGTACTGCGGTGATCAACGCCGCCAGCGGTCGTCAGACTGTCGATGAAGCCCTG |
| | AAAGACGCGCAGACTCCCATGAGCGATTACGACATCCCCACTACTGAGAATCTTTA |
| | TTTTCAGGGCGCCATGCCGGAGCCTCATGCTGTGTTGTACGTTACAAATGAACTTT |
| | CACACATTGTTAAGGATGGCTTTTTACCAATCTGGAAACTTACCGGCGATGAGTCC |
| | CTTAACGATTTGTGGTTAGAGAACGGTAAATACGCCACGGACGTTTATGCCTACGG |
| | TGACGTATCGAAGTGGACCATTCGCCAGCTGCGCGGTCATGGGTTTATTTTCATCT |
| | CGACACACAAGAACGTCCAACTCGCGGATATCATTAAGACTGTGGACGTTCGGATC |
| | CCTCGTGAGGTGGCCCGCTCTCATGACATGAAGGCGTTTGAAAATGAAATTGGGC |
| | GTCGCCGTATTCGGATGCGTAAAGGCTTTGGTGATGCATTACGTAATTACGCCTTT |
| | AAGATGGCAATTGAATTTCATGGTAGCGAGGCTGAAACTCTGAACGACGCAAATCC |
| | TCGTCTTCACAAAATCTATGGGATGCCGGAAATCCCGCCACTTTACATGGAATACG |
| | CGGAAATCGGGACACGTTTCGATGATGAGCCGACTGACGAGAAACTTGTATCTATG |
| | CTTGACTACATTGTCTATAGCGCCGAAGAAGTTCACTACATCGGGTGCGGGGACTT |
| | ACGGACGCTTATGCAGTTTAAAAAACGTTCACCGGGTCGGTTTCGGCGTGTGTTGT |
| | GGCATGTATACGACCCGATTGCACCGGAGTGCTCAGATCCGAATGTTATCGTTCAT |
| | AATATCATGGTTGACTCCAAGAAAGACATTTTGAAGCATATGAATTTTTTAAAGCGC |
| | GTTGAGCGCTTATTCATCTGGGATGTCTCCAGTGACCGGAGTCAGATGAATGATCA |
| | CGAATGGGAAACAACGCGCTTCGCAGAGGATCGGCTGGGTGAGGAAATCGCCTAT |
| | GAGATGGGGGGTGCGTTCTCTTCGGCTTTAATTAAGCACCGCATCCCAAACTCGAA |
| | AGACGAGTATCATTGTATTAGCACTTACCTGTTTCCACAACCGGGGGCTGACGCGG |
| | ACATGTACGAATTGCGTAATTTTATGCGTCTTCGTGGGTACTCACACGTTGATCGCC |
| | ACATGCATCCGGATGCTTCGGTAACAAAGGTCGTGTCGCGGGATGTGCGTAAAAT |
| | GGTCGAGCTTTACCACGGTCGTGATCGCGGCCGGTTCCTCAAAAAACGCTTGTTTG |
| | AGCATTTACATATCGTGCGCAAGAATGGGCTCTTGCACGAATCGGACGAGCCACG |
| | GGCGGATTTATTCTACCTTACTAATCGGTGTAACATGGGTCTGGAGCCTTCGATTTA |
| | CGAAGTCATGAAAAAATCAGTCATCGCCACGGCATGGGTCGGCCGTGCCCCTCTG |
| | TATGACTATGATGACTTTGCTCTCCCGCGCAGCACAGTTATGTTAAATGGCAGCTA |
| | CCGCGATATCCGGATTCTGGATGGCAACGGCGCAATCCTTTTCCTCATGTGGCGTT |
| | ATCCTGATATTGTCAAGAAGGACTTAACATACGACCCGGCTTGGGCAATGAACTTC |
| | GCTGTATCACTTAAAGAACCAATTCCGGATCCGCCTGTGCCGGACATCAGTTTATG |
| | TCGGTTTATTGGCCTCCGCGTCGAATCATCCGTTTTGCGCGTACGTAATCCAACGC |
| | TTCACGAAACCGCCGATGAGTTAAAACGTATGGGTCTGGATCTGTCGGGCCATTTA |
| | TACGTGACTCTCATGAGTGGTGCTTACGTGACAGACCTCTTTTGGTGGTTCAAAAT |
| | GATCCTGGACTGGAGCGCTCAAAATCGGGAACAGAAGCTTCGGGACCTTAAACGC |
| | TCGGCCGCTGAGGTTATCGAATGGAAGGAGCAGATGGCAGAGCGGCCATGGCAT |
| | GTACGCAACGACCTGATTGCTGCACTTCGCGAGTACAAGCGTAAAATGGGTATGCG |
| | GGAGGGGGCGTCTATCGACTCGTGGCTGGAGTTACTGCGCCATTTA |
| SEQ ID NO: | ATGAAACATCACCATCACCATCACGGCAGCCTGCAAGAAGAGAAACCGAAAGAGG |
| 35 | GCGTTAAGACCGAGAATGACCACATTAACCTGAAGGTCGCTGGTCAAGATGGCAG |
| His6-SUMO- | CGTGGTGCAGTTTAAGATCAAGCGTCACACGCCGTTGAGCAAGCTGATGAAGGCTT |
| VP4 | ACTGCGAGCGTCAGGGTCTGAGCATGCGTCAGATCCGCTTTCGTTTCGATGGCCA |
| sequence | GCCGATCAATGAGACTGACACCCCAGCGCAACTGGAGATGGAAGATGAAGATACC |
| codon | ATCGACGTCTTTCAGCAACAGACCGGTGGTCCCATGAGCGATTACGACATCCCCAC |
| optimized | TACTGAGAATCTTTATTTTCAGGGCGCCATGCCGGAGCCTCATGCTGTGTTGTACG |
| according to | TTACAAATGAACTTTCACACATTGTTAAGGATGGCTTTTTACCAATCTGGAAACTTAC |
| method A | CGGCGATGAGTCCCTTAACGATTTGTGGTTAGAGAACGGTAAATACGCCACGGAC |
| | GTTTATGCCTACGGTGACGTATCGAAGTGGACCATTCGCCAGCTGCGCGGTCATG |
| | GGTTTATTTTCATCTCGACACACAAGAACGTCCAACTCGCGGATATCATTAAGACTG |
| | TGGACGTTCGGATCCCTCGTGAGGTGGCCCGCTCTCATGACATGAAGGCGTTTGA |
| | AAATGAAATTGGGCGTCGCCGTATTCGGATGCGTAAAGGCTTTGGTGATGCATTAC |
| | GTAATTACGCCTTTAAGATGGCAATTGAATTTCATGGTAGCGAGGCTGAAACTCTGA |
| | ACGACGCAAATCCTCGTCTTCACAAAATCTATGGGATGCCGGAAATCCCGCCACTT |
| | TACATGGAATACGCGGAAATCGGGACACGTTTCGATGATGAGCCGACTGACGAGA |
| | AACTTGTATCTATGCTTGACTACATTGTCTATAGCGCCGAAGAAGTTCACTACATCG |
| | GGTGCGGGGACTTACGGACGCTTATGCAGTTTAAAAAACGTTCACCGGGTCGGTTT |
| | CGGCGTGTGTTGTGGCATGTATACGACCCGATTGCACCGGAGTGCTCAGATCCGA |
| | ATGTTATCGTTCATAATATCATGGTTGACTCCAAGAAAGACATTTTGAAGCATATGA |
| | ATTTTTTAAAGCGCGTTGAGCGCTTATTCATCTGGGATGTCTCCAGTGACCGGAGT |
| | CAGATGAATGATCACGAATGGGAAACAACGCGCTTCGCAGAGGATCGGCTGGGTG |
| | AGGAAATCGCCTATGAGATGGGGGGTGCGTTCTCTTCGGCTTTAATTAAGCACCGC |
| | ATCCCAAACTCGAAAGACGAGTATCATTGTATTAGCACTTACCTGTTTCCACAACCG |
| | GGGGCTGACGCGGACATGTACGAATTGCGTAATTTTATGCGTCTTCGTGGGTACTC |
| | ACACGTTGATCGCCACATGCATCCGGATGCTTCGGTAACAAAGGTCGTGTCGCGG |
| | GATGTGCGTAAAATGGTCGAGCTTTACCACGGTCGTGATCGCGGCCGGTTCCTCA |
| | AAAAACGCTTGTTTGAGCATTTACATATCGTGCGCAAGAATGGGCTCTTGCACGAA |
| | TCGGACGAGCCACGGGGGGATTTATTCTACCTTACTAATCGGTGTAACATGGGTCT |
| | GGAGCCTTCGATTTACGAAGTCATGAAAAAATCAGTCATCGCCACGGCATGGGTCG |
| | GCCGTGCCCCTCTGTATGACTATGATGACTTTGCTCTCCCGCGCAGCACAGTTATG |
| | TTAAATGGCAGCTACCGCGATATCCGGATTCTGGATGGCAACGGCGCAATCCTTTT |
| | CCTCATGTGGCGTTATCCTGATATTGTCAAGAAGGACTTAACATACGACCCGGCTT |
| | GGGCAATGAACTTCGCTGTATCACTTAAAGAACCAATTCCGGATCCGCCTGTGCCG |
| | GACATCAGTTTATGTCGGTTTATTGGCCTCCGCGTCGAATCATCCGTTTTGCGCGT |
| | ACGTAATCCAACGCTTCACGAAACCGCCGATGAGTTAAAACGTATGGGTCTGGATC |
| | TGTCGGGCCATTTATACGTGACTCTCATGAGTGGTGCTTACGTGACAGACCTCTTT |
| | TGGTGGTTCAAAATGATCCTGGACTGGAGCGCTCAAAATCGGGAACAGAAGCTTCG |
| | GGACCTTAAACGCTCGGCCGCTGAGGTTATCGAATGGAAGGAGCAGATGGCAGAG |
| | CGGCCATGGCATGTACGCAACGACCTGATTGCTGCACTTCGCGAGTACAAGCGTA |
| | AAATGGGTATGCGGGAGGGGGCGTCTATCGACTCGTGGCTGGAGTTACTGCGCCA |
| | TTTA |
| SEQ ID NO: | ATGCCTGAACCCCACGCCGTCTTATACGTAACTAACGAGTTATCTCATATTGTAAAA |
| 36 | AACGGGTTTCTTCCCATTTGGAAACTTACCGGCGATGAGTCTTTGAATGACCTGTG |
| VP4-noTEV- | GCTTGAAAATGGAAAATACGCCACCGACGTATACGCATACGGAGATGTATCTAAAT |
| Fh8-His6 | GGACGATCCGCCAGTTGCGCGGACACGGGTTCATTTTCATTAGCACGCATAAAAAC |
| optimized | GTGCAGCTGGCAGACATTATTAAGACTGTGGACGTCCGTATCCCTCGCGAGGTAG |
| according to | CACGCTCGCATGATATGAAAGCATTCGAAAATGAAATCTCACGTCGCCGTATCCGC |
| method B | ATGCGCAAAGGATTCGGTGACGCGCTGCGTAACTATGCGTTTAAAATGGCGATTGA |
| | GTTTCATGGCAGCGAAGCCGAAACGTTGAACGACGCCAACCCACGTCTTCACAAG |
| | ATTTACGGGATGCCCGAAATCCCTCCCTTGTACATGGAGTACGCCGAGATCGGTAC |
| | TCGTTTCGATGACGAACCCACAGATGAAAAGTTGGTTAGTATGCTTGACTACATTGT |
| | TTATTCCGCGGAGGAGGTTCATTACGTGGGCTGCGGAGACTTACGCACACTTATGC |
| | AGTTCAAGAAGCGCTCTCCTGGCCGCTTTCGCCGCGTATTGTGGCATGTGTACGAC |
| | CCCATCGCGCCCGAATGTAGCGATCCTAATGTAATTGTACATAACATCATGGTCGA |
| | TTCCAAAAAAGATATTCTTAAACATATGAACTTCTTAAAACGTGTGGAGCGCCTTTTC |
| | ATTTGGGATGTCAGCTCGGATCGCTCTCAGATGAACGATCACGAGTGGGAAACGA |
| | CGCGTTTCGCCGAGGACCGTTTAGGTGAGGAAATCGCGTACGAGATGGGCGGGG |
| | CTTTCTCATCGGCTTTGATTAAGCACCGCATCCCAAACAGCAAGGACGAATATCAC |
| | TGTATTTCGACCTACCTTTTCCCCCAACCGGGAGCAGACGCAGACATGTACGAGCT |
| | GCGCAACTTCATGCGCTTACGTGGGTATTCACATGTCGACCGTCATATGCACCCAG |
| | ACGCTTCAGTGACTAAAGTAGTGAGTCGTGACGTTCGCAAGATGGTGGAATTGTAC |
| | CACGGCCGTGACCGTGGTCGTTTCTTAAAGAAACGCTTATTTGAACATCTGCACAT |
| | CGTCCGTAAGAATGGGCTGCTTCACGAATCGGACGAGCCACGTGCGGACTTATTC |
| | TATTTGACAAACCGCTGCAACATGGGTTTGGAGCCCAGTATCTATGAAGTGATGAA |
| | GAAATCCGTCATTGCCACCGCATGGGTGGGTCGTGCGCCTCTTTATGACTATGACG |
| | ATTTTGCATTGCCGCGTTCAACCGTTATGTTAAACGGTAGTTACCGCGATATCCGTA |
| | TTTTGGACGGGAACGGCGCGATCTTATTTCTTATGTGGCGCTATCCCGATATTGTC |
| | AAGAAGGACCTGACATATGACCCCGCGTGGGCCATGAATTTTGCGGTCAGCTTAAA |
| | AGAGCCTATCCCGGATCCACCTGTTCCAGACATCAGTTTATGCCGCTTTATCGGGT |
| | TACGCGTCGAATCGAGCGTTCTTCGCGTTCGTAATCCAACGTTGCATGAGACGGCT |
| | GACGAGTTGAAACGTATGGGCTTAGACCTTTCTGGCCATCTGTACGTCACCTTGAT |
| | GAGCGGGGCATATGTGACCGACCTTTTTTGGTGGTTTAAGATGATTTTAGATTGGT |
| | CTGCGCAAAACAAGGAGCAGAAGCTGCGTGACCTTAAGCGCTCAGCGGCGGAGGT |
| | AATCGAGTGGAAAGAACAGATGGCCGAACGTCCGTGGCACGTCCGTAACGACCTT |
| | ATCCGCGCTCTGCGCGAATACAAACGCAAGATGGGCATGCGTGAAGGTGCATCGA |
| | TCGACTCGTGGCTGGAATTGCTTCGCCACCTTATGAGCGATTACGACATCGACACT |
| | ACTATGCCCTCCGTCCAGGAGGTCGAAAAATTATTACATGTGTTAGACCGTAACGG |
| | AGATGGAAAAGTTAGCGCCGAGGAGCTGAAGGCATTCGCGGACGACTCCAAGTGC |
| | CCTCTTGACTCGAACAAGATTAAAGCATTTATTAAAGAACACGATAAGAATAAAGAC |
| | GGGAAGTTGGACTTGAAGGAATTGGTATCGATCTTATCGAGTATGAAACACCACCA |
| | TCACCATCAC |
| SEQ ID NO: | ATGAAACATCACCATCACCATCACATGCCGTCCGTTCAAGAAGTCGAAAAACTTCT |
| 37 | GCATGTTCTGGATCGTAACGGCGATGGAAAAGTATCCGCCGAGGAATTGAAGGCA |
| His6-Fh8- | TTTGCTGACGATTCGAAATGTCCGTTGGATAGTAATAAGATCAAAGCATTCATCAAA |
| noTEV-VP4 | GAGCATGACAAGAACAAGGACGGGAAATTGGATTTGAAGGAGTTGGTGTCTATTCT |
| sequence | TAGCTCTATGCCGGAGCCTCATGCTGTGTTGTACGTTACAAATGAACTTTCACACAT |
| codon | TGTTAAGGATGGCTTTTTACCAATCTGGAAACTTACCGGCGATGAGTCCCTTAACG |
| optimized | ATTTGTGGTTAGAGAACGGTAAATACGCCACGGACGTTTATGCCTACGGTGACGTA |
| according to | TCGAAGTGGACCATTCGCCAGCTGCGCGGTCATGGGTTTATTTTCATCTCGACACA |
| method B | CAAGAACGTCCAACTCGCGGATATCATTAAGACTGTGGACGTTCGGATCCCTCGTG |
| | AGGTGGCCCGCTCTCATGACATGAAGGCGTTTGAAAATGAAATTGGGCGTCGCCG |
| | TATTCGGATGCGTAAAGGCTTTGGTGATGCATTACGTAATTACGCCTTTAAGATGGC |
| | AATTGAATTTCATGGTAGCGAGGCTGAAACTCTGAACGACGCAAATCCTCGTCTTC |
| | ACAAAATCTATGGGATGCCGGAAATCCCGCCACTTTACATGGAATACGCGGAAATC |
| | GGGACACGTTTCGATGATGAGCCGACTGACGAGAAACTTGTATCTATGCTTGACTA |
| | CATTGTCTATAGCGCCGAAGAAGTTCACTACATCGGGTGCGGGGACTTACGGACG |
| | CTTATGCAGTTTAAAAAACGTTCACCGGGTCGGTTTCGGCGTGTGTTGTGGCATGT |
| | ATACGACCCGATTGCACCGGAGTGCTCAGATCCGAATGTTATCGTTCATAATATCAT |
| | GGTTGACTCCAAGAAAGACATTTTGAAGCATATGAATTTTTTAAAGCGCGTTGAGCG |
| | CTTATTCATCTGGGATGTCTCCAGTGACCGGAGTCAGATGAATGATCACGAATGGG |
| | AAACAACGCGCTTCGCAGAGGATCGGCTGGGTGAGGAAATCGCCTATGAGATGGG |
| | GGGTGCGTTCTCTTCGGCTTTAATTAAGCACCGCATCCCAAACTCGAAAGACGAGT |
| | ATCATTGTATTAGCACTTACCTGTTTCCACAACCGGGGGCTGACGCGGACATGTAC |
| | GAATTGCGTAATTTTATGCGTCTTCGTGGGTACTCACACGTTGATCGCCACATGCAT |
| | CCGGATGCTTCGGTAACAAAGGTCGTGTCGCGGGATGTGCGTAAAATGGTCGAGC |
| | TTTACCACGGTCGTGATCGCGGCCGGTTCCTCAAAAAACGCTTGTTTGAGCATTTA |
| | CATATCGTGCGCAAGAATGGGCTCTTGCACGAATCGGACGAGCCACGGGGGGATT |
| | TATTCTACCTTACTAATCGGTGTAACATGGGTCTGGAGCCTTCGATTTACGAAGTCA |
| | TGAAAAAATCAGTCATCGCCACGGCATGGGTCGGCCGTGCCCCTCTGTATGACTAT |
| | GATGACTTTGCTCTCCCGCGCAGCACAGTTATGTTAAATGGCAGCTACCGCGATAT |
| | CCGGATTCTGGATGGCAACGGCGCAATCCTTTTCCTCATGTGGCGTTATCCTGATA |
| | TTGTCAAGAAGGACTTAACATACGACCCGGCTTGGGCAATGAACTTCGCTGTATCA |
| | CTTAAAGAACCAATTCCGGATCCGCCTGTGCCGGACATCAGTTTATGTCGGTTTATT |
| | GGCCTCCGCGTCGAATCATCCGTTTTGCGCGTACGTAATCCAACGCTTCACGAAAC |
| | CGCCGATGAGTTAAAACGTATGGGTCTGGATCTGTCGGGCCATTTATACGTGACTC |
| | TCATGAGTGGTGCTTACGTGACAGACCTCTTTTGGTGGTTCAAAATGATCCTGGAC |
| | TGGAGCGCTCAAAATCGGGAACAGAAGCTTCGGGACCTTAAACGCTCGGCCGCTG |
| | AGGTTATCGAATGGAAGGAGCAGATGGCAGAGCGGCCATGGCATGTACGCAACGA |
| | CCTGATTGCTGCACTTCGCGAGTACAAGCGTAAAATGGGTATGCGGGAGGGGGCG |
| | TCTATCGACTCGTGGCTGGAGTTACTGCGCCATTTA |
| SEQ ID NO: | ATGAAGCAAAGCACTATTGCACTGGCACTCTTACCGTTACTGTTTACCCCTGTGACA |
| 42 | AAAGCAATGAAACATCACCATCACCATCACCCCATGAGCGATTACGACATCCCCAC |
| PhoAE- | TACTGAGAATCTTTATTTTCAGGGCGCCATGCCGGAGCCTCATGCTGTGTTGTACG |
| His6-VP4 | TTACAAATGAACTTTCACACATTGTTAAGGATGGCTTTTTACCAATCTGGAAACTTAC |
| sequence | CGGCGATGAGTCCCTTAACGATTTGTGGTTAGAGAACGGTAAATACGCCACGGAC |
| codon | GTTTATGCCTACGGTGACGTATCGAAGTGGACCATTCGCCAGCTGCGCGGTCATG |
| optimized | GGTTTATTTTCATCTCGACACACAAGAACGTCCAACTCGCGGATATCATTAAGACTG |
| according to | TGGACGTTCGGATCCCTCGTGAGGTGGCCCGCTCTCATGACATGAAGGCGTTTGA |
| method A | AAATGAAATTGGGCGTCGCCGTATTCGGATGCGTAAAGGCTTTGGTGATGCATTAC |
| | GTAATTACGCCTTTAAGATGGCAATTGAATTTCATGGTAGCGAGGCTGAAACTCTGA |
| | ACGACGCAAATCCTCGTCTTCACAAAATCTATGGGATGCCGGAAATCCCGCCACTT |
| | TACATGGAATACGCGGAAATCGGGACACGTTTCGATGATGAGCCGACTGACGAGA |
| | AACTTGTATCTATGCTTGACTACATTGTCTATAGCGCCGAAGAAGTTCACTACATCG |
| | GGTGCGGGGACTTACGGACGCTTATGCAGTTTAAAAAACGTTCACCGGGTCGGTTT |
| | CGGCGTGTGTTGTGGCATGTATACGACCCGATTGCACCGGAGTGCTCAGATCCGA |
| | ATGTTATCGTTCATAATATCATGGTTGACTCCAAGAAAGACATTTTGAAGCATATGA |
| | ATTTTTTAAAGCGCGTTGAGCGCTTATTCATCTGGGATGTCTCCAGTGACCGGAGT |
| | CAGATGAATGATCACGAATGGGAAACAACGCGCTTCGCAGAGGATCGGCTGGGTG |
| | AGGAAATCGCCTATGAGATGGGGGGTGCGTTCTCTTCGGCTTTAATTAAGCACCGC |
| | ATCCCAAACTCGAAAGACGAGTATCATTGTATTAGCACTTACCTGTTTCCACAACCG |
| | GGGGCTGACGCGGACATGTACGAATTGCGTAATTTTATGCGTCTTCGTGGGTACTC |
| | ACACGTTGATCGCCACATGCATCCGGATGCTTCGGTAACAAAGGTCGTGTCGCGG |
| | GATGTGCGTAAAATGGTCGAGCTTTACCACGGTCGTGATCGCGGCCGGTTCCTCA |
| | AAAAACGCTTGTTTGAGCATTTACATATCGTGCGCAAGAATGGGCTCTTGCACGAA |
| | TCGGACGAGCCACGGGGGGATTTATTCTACCTTACTAATCGGTGTAACATGGGTCT |
| | GGAGCCTTCGATTTACGAAGTCATGAAAAAATCAGTCATCGCCACGGCATGGGTCG |
| | GCCGTGCCCCTCTGTATGACTATGATGACTTTGCTCTCCCGCGCAGCACAGTTATG |
| | TTAAATGGCAGCTACCGCGATATCCGGATTCTGGATGGCAACGGCGCAATCCTTTT |
| | CCTCATGTGGCGTTATCCTGATATTGTCAAGAAGGACTTAACATACGACCCGGCTT |
| | GGGCAATGAACTTCGCTGTATCACTTAAAGAACCAATTCCGGATCCGCCTGTGCCG |
| | GACATCAGTTTATGTCGGTTTATTGGCCTCCGCGTCGAATCATCCGTTTTGCGCGT |
| | ACGTAATCCAACGCTTCACGAAACCGCCGATGAGTTAAAACGTATGGGTCTGGATC |
| | TGTCGGGCCATTTATACGTGACTCTCATGAGTGGTGCTTACGTGACAGACCTCTTT |
| | TGGTGGTTCAAAATGATCCTGGACTGGAGCGCTCAAAATCGGGAACAGAAGCTTCG |
| | GGACCTTAAACGCTCGGCCGCTGAGGTTATCGAATGGAAGGAGCAGATGGCAGAG |
| | CGGCCATGGCATGTACGCAACGACCTGATTGCTGCACTTCGCGAGTACAAGCGTA |
| | AAATGGGTATGCGGGAGGGGGCGTCTATCGACTCGTGGCTGGAGTTACTGCGCCA |
| | TTTATAA |

1. A fusion protein comprising a messenger RNA (mRNA) capping enzyme polypeptide linked to a Fh8 polypeptide or fragment thereof.

2. The fusion protein of claim 1, wherein the Fh8 polypeptide or fragment thereof comprises an amino acid sequence comprising at least 90% identity to an amino acid sequence set forth in SEQ ID NO: 10.

3. The fusion protein of claim 1 or 2, wherein the Fh8 polypeptide or fragment thereof is linked to the N-terminus or the C-terminus of the capping enzyme polypeptide.

4. The fusion protein of any one of claims 1 to 3, wherein the capping enzyme polypeptide comprises a vaccina virus D1 subunit, optionally wherein:
the vaccina virus D1 subunit comprises an amino acid sequence comprising at least 90% identity to an amino acid sequence set forth in SEQ ID NO: 1; and/or
the fusion protein comprises an amino acid sequence comprising at least 90% identity to an amino acid sequence set forth in SEQ ID NO: 3.

5. The fusion protein of any one of claims 1 to 4, wherein the capping enzyme polypeptide comprises a vaccina virus D12 subunit, optionally wherein the vaccina virus D12 subunit comprises an amino acid sequence comprising at least 90% identity to an amino acid sequence set forth in SEQ ID NO: 2.

6. The fusion protein of any one of claims 1 to 3, wherein the capping enzyme polypeptide comprises a vaccina virus VP39 polypeptide or fragment thereof, optionally wherein:
the VP39 polypeptide comprises an amino acid sequence comprising at least 90% identity to an amino acid sequence set forth in SEQ ID NO: 6, and/or wherein the fusion protein comprises an amino acid sequence comprising at least 90% identity to an amino acid sequence set forth in SEQ ID NO: 4; or
the VP39 polypeptide fragment comprises an amino acid sequence comprising at least 90% identity to an amino acid sequence set forth in SEQ ID NO: 7, and/or wherein the fusion protein comprises an amino acid sequence comprising at least 90% identity to an amino acid sequence set forth in SEQ ID NO: 5.

7. The fusion protein of any one of claims 1 to 3, wherein the capping enzyme polypeptide comprises a bluetongue virus VP4 polypeptide or fragment thereof, optionally wherein:
the VP4 polypeptide comprises an amino acid sequence comprising at least 90% identity to an amino acid sequence set forth in SEQ ID NO: 16; and/or
the fusion protein comprises an amino acid sequence comprising at least 90% identity to an amino acid sequence set forth in SEQ ID NO: 17, SEQ ID NO: 18, or SEQ ID NO: 22.

8. A polynucleotide comprising a nucleotide sequence that encodes the fusion protein according to any one of claims 1 to 7, optionally the nucleotide sequence is codon optimized.

9. The polynucleotide of claim 8, wherein the nucleotide sequence comprises at least 90% identity to a nucleotide sequence set forth in SEQ ID NO: 23, SEQ ID NO: 24, SEQ ID NO: 32, SEQ ID NO: 33, or SEQ ID NO: 37.

10. An expression vector comprising the polynucleotide of claim 8 or 9.

11. A host cell comprising the expression vector of claim 10, optionally wherein the host cell is an E. coli cell, optionally wherein the E. coli cell is a BL21 (DE3) or Origami E. coli cell strain.

12. A method of expressing a fusion protein, comprising culturing the host cell of claim 11 under conditions sufficient to express the fusion protein, optionally wherein the fusion protein is further isolated from the host cell.

13. A method of capping an mRNA, comprising:
a) incubating the mRNA with the fusion protein of any one of claims 1-5 under conditions sufficient to cap the mRNA with a cap0 structure,
b) incubating the mRNA with the fusion protein of claim 4 or 5 and after or simultaneously with the fusion protein of claim 6 under conditions sufficient to cap the mRNA with a cap1 structure, or
c) incubating the mRNA with the fusion protein of any one of claim 1-3 or 7 under conditions sufficient to cap the mRNA with a cap1 structure.

14. A method of converting a cap0 structure on an mRNA to a cap1 structure, comprising incubating the mRNA with the fusion protein of any one of claim 1-3 or 6 under conditions sufficient to cap the mRNA.

15. A process of preparing an mRNA comprising a step of capping, comprising:
a) incubating the mRNA with the fusion protein of any one of claims 1-5 under conditions sufficient for the mRNA to be capped with a cap0 structure,
b) incubating the mRNA capped with a cap0 structure with the fusion protein of claim 6 under conditions sufficient for the mRNA to be capped with a cap1 structure,
d) optionally purifying the capped mRNA,
e) optionally tailing the mRNA with a polyadenylation step, and
f) optionally purifying the capped polyadenylated mRNA.

16. A process of preparing an mRNA comprising a step of capping, comprising:
a) incubating the mRNA with the fusion protein of any one of claim 1-3 or 7 under conditions sufficient for the mRNA to be capped with a cap1 structure,
b) optionally purifying the capped mRNA,
c) optionally tailing the mRNA with a polyadenylation step, and
d) optionally purifying the capped polyadenylated mRNA.

17. A capped mRNA obtained by the method of claim 13 or 14, or by the process of claim 15 or 16.


SEQUENCE LISTING (43 sequences)

SEQ ID NO: 1 - protein, 844 aa, Vaccinia virus
MDANVVSSSTIATYIDALAKNASELEQRSTAYEINNELELVFIKPPLITLTNVVNISTIQESFIRFTVTNKEGVKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDELIKELTTLSRHIFMASPENVILSPPINAPIKTFMLPKQDIVGLDLENLYAVTKTDGIPITIRVTSNGLYCYFTHLGYIIRYPVKRIIDSEVVVFGEAVKDKNWTVYLIKLIEPVNAINDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFVEYKKFSNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDIFNEDKLSDVGHQYANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 2 - protein, 287 aa, Vaccinia virus
MDEIVKNIREGTHVLLPFYETLPELNLSLGKSPLPSLEYGANYFLQISRVNDLNRMPTDMLKLFTHDIMLPESDLDKVYEILKINSVKYYGRSTKADAVVADLSARNKLFKRERDAIKSNNHLTENNLYISDYKMLTFDVFRPLFDFVNEKYCIIKLPTLFGRGVIDTMRIYCSLFKNVRLLKCVSDSWLKDSAIMVASDVCKKNLDLFMSHVKSVTKSSSWKDVNSVQFSILNNPVDTEFINKFLEFSNRVYEALYYVHSLLYSSMTSDSKSIENKHQRRLVKLLL

SEQ ID NO: 3 - protein, 913 aa, synthetic construct
MPSVQEVEKLLHVLDRNGDGKVSAEELKAFADDSKCPLDSNKIKAFIKEHDKNKDGKLDLKELVSILSSMDANVVSSSTIATYIDALAKNASELEQRSTAYEINNELELVFIKPPLITLTNVVNISTIQESFIRFTVTNKEGVKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDELIKELTTLSRHIFMASPENVILSPPINAPIKTFMLPKQDIVGLDLENLYAVTKTDGIPITIRVTSNGLYCYFTHLGYIIRYPVKRIIDSEVVVFGEAVKDKNWTVYLIKLIEPVNAINDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFVEYKKFSNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDIFNEDKLSDVGHQYANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 4 - protein, 402 aa, synthetic construct
MPSVQEVEKLLHVLDRNGDGKVSAEELKAFADDSKCPLDSNKIKAFIKEHDKNKDGKLDLKELVSILSSMDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTEKVSHEPIQRKISSKNSMSKNRNSKRSVRSNK

SEQ ID NO: 5 - protein, 376 aa, synthetic construct
MPSVQEVEKLLHVLDRNGDGKVSAEELKAFADDSKCPLDSNKIKAFIKEHDKNKDGKLDLKELVSILSSMDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTEKVSHE

SEQ ID NO: 6 - protein, 333 aa, Vaccinia virus
MDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTEKVSHEPIQRKISSKNSMSKNRNSKRSVRSNK

SEQ ID NO: 7 - protein, 307 aa, synthetic construct
MDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTEKVSHE

SEQ ID NO: 8 - protein, 218 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 9 - protein, 90 aa, synthetic construct
EEKPKEGVKTENDHINLKVAGQDGSVVQFKIKRHTPLSKLMKAYCERQGLSMRQIRFRFDGQPINETDTPAQLEMEDEDTIDVFQQQTGG

SEQ ID NO: 10 - protein, 69 aa, Fasciola hepatica
MPSVQEVEKLLHVLDRNGDGKVSAEELKAFADDSKCPLDSNKIKAFIKEHDKNKDGKLDLKELVSILSS

SEQ ID NO: 11 - protein, 25 aa, synthetic construct
MMITLRKLPLAVAVAAGVMSAQAMA

SEQ ID NO: 12 - protein, 28 aa, synthetic construct
MFKFKKKFLVGLTAAFMSISMFSATASA

SEQ ID NO: 13 - protein, 21 aa, synthetic construct
MKQSTIALALLPLLFTPVTKA

SEQ ID NO: 14 - protein, 21 aa, synthetic construct
MKYLLPAAAGLLLLAAQPAMA

SEQ ID NO: 15 - protein, 26 aa, synthetic construct
MKIKTGARILALSALTTMMFSASALA

SEQ ID NO: 16 - protein, 644 aa, Bluetongue virus
MPEPHAVLYVTNELSHIVKNGFLPIWKLTGDESLNDLWLENGKYATDVYAYGDVSKWTIRQLRGHGFIFISTHKNVQLADIIKTVDVRIPREVARSHDMKAFENEISRRRIRMRKGFGDALRNYAFKMAIEFHGSEAETLNDANPRLHKIYGMPEIPPLYMEYAEIGTRFDDEPTDEKLVSMLDYIVYSAEEVHYVGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNKEQKLRDLKRSAAEVIEWKEQMAERPWHVRNDLIRALREYKRKMGMREGASIDSWLELLRHL

SEQ ID NO: 17 - protein, 722 aa, synthetic construct
MPEPHAVLYVTNELSHIVKNGFLPIWKLTGDESLNDLWLENGKYATDVYAYGDVSKWTIRQLRGHGFIFISTHKNVQLADIIKTVDVRIPREVARSHDMKAFENEISRRRIRMRKGFGDALRNYAFKMAIEFHGSEAETLNDANPRLHKIYGMPEIPPLYMEYAEIGTRFDDEPTDEKLVSMLDYIVYSAEEVHYVGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNKEQKLRDLKRSAAEVIEWKEQMAERPWHVRNDLIRALREYKRKMGMREGASIDSWLELLRHLMSDYDIDTTMPSVQEVEKLLHVLDRNGDGKVSAEELKAFADDSKCPLDSNKIKAFIKEHDKNKDGKLDLKELVSILSS

SEQ ID NO: 18 - protein, 731 aa, synthetic construct
MPEPHAVLYVTNELSHIVKNGFLPIWKLTGDESLNDLWLENGKYATDVYAYGDVSKWTIRQLRGHGFIFISTHKNVQLADIIKTVDVRIPREVARSHDMKAFENEISRRRIRMRKGFGDALRNYAFKMAIEFHGSEAETLNDANPRLHKIYGMPEIPPLYMEYAEIGTRFDDEPTDEKLVSMLDYIVYSAEEVHYVGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNKEQKLRDLKRSAAEVIEWKEQMAERPWHVRNDLIRALREYKRKMGMREGASIDSWLELLRHLPMSDYDIPTTENLYFQGAMPSVQEVEKLLHVLDRNGDGKVSAEELKAFADDSKCPLDSNKIKAFIKEHDKNKDGKLDLKELVSILSS

SEQ ID NO: 19 - protein, 1035 aa, synthetic construct
MKHHHHHHIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPMSDYDIPTTENLYFQGAMPEPHAVLYVTNELSHIVKDGFLPIWKLTGDESLNDLWLENGKYATDVYAYGDVSKWTIRQLRGHGFIFISTHKNVQLADIIKTVDVRIPREVARSHDMKAFENEIGRRRIRMRKGFGDALRNYAFKMAIEFHGSEAETLNDANPRLHKIYGMPEIPPLYMEYAEIGTRFDDEPTDEKLVSMLDYIVYSAEEVHYIGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNREQKLRDLKRSAAEVIEWKEQMAERPWHVRNDLIAALREYKRKMGMREGASIDSWLELLRHL

SEQ ID NO: 20 - protein, 764 aa, synthetic construct
MKHHHHHHGSLQEEKPKEGVKTENDHINLKVAGQDGSVVQFKIKRHTPLSKLMKAYCERQGLSMRQIRFRFDGQPINETDTPAQLEMEDEDTIDVFQQQTGGPMSDYDIPTTENLYFQGAMPEPHAVLYVTNELSHIVKDGFLPIWKLTGDESLNDLWLENGKYATDVYAYGDVSKWTIRQLRGHGFIFISTHKNVQLADIIKTVDVRIPREVARSHDMKAFENEIGRRRIRMRKGFGDALRNYAFKMAIEFHGSEAETLNDANPRLHKIYGMPEIPPLYMEYAEIGTRFDDEPTDEKLVSMLDYIVYSAEEVHYIGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNREQKLRDLKRSAAEVIEWKEQMAERPWHVRNDLIAALREYKRKMGMREGASIDSWLELLRHL

SEQ ID NO: 21 - protein, 730 aa, synthetic construct
MPEPHAVLYVTNELSHIVKNGFLPIWKLTGDESLNDLWLENGKYATDVYAYGDVSKWTIRQLRGHGFIFISTHKNVQLADIIKTVDVRIPREVARSHDMKAFENEISRRRIRMRKGFGDALRNYAFKMAIEFHGSEAETLNDANPRLHKIYGMPEIPPLYMEYAEIGTRFDDEPTDEKLVSMLDYIVYSAEEVHYVGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNKEQKLRDLKRSAAEVIEWKEQMAERPWHVRNDLIRALREYKRKMGMREGASIDSWLELLRHLMSDYDIDTTMPSVQEVEKLLHVLDRNGDGKVSAEELKAFADDSKCPLDSNKIKAFIKEHDKNKDGKLDLKELVSILSSMKHHHHHH

SEQ ID NO: 22 - protein, 713 aa, synthetic construct
MPSVQEVEKLLHVLDRNGDGKVSAEELKAFADDSKCPLDSNKIKAFIKEHDKNKDGKLDLKELVSILSSMPEPHAVLYVTNELSHIVKDGFLPIWKLTGDESLNDLWLENGKYATDVYAYGDVSKWTIRQLRGHGFIFISTHKNVQLADIIKTVDVRIPREVARSHDMKAFENEIGRRRIRMRKGFGDALRNYAFKMAIEFHGSEAETLNDANPRLHKIYGMPEIPPLYMEYAEIGTRFDDEPTDEKLVSMLDYIVYSAEEVHYIGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNREQKLRDLKRSAAEVIEWKEQMAERPWHVRNDLIAALREYKRKMGMREGASIDSWLELLRHL

SEQ ID NO: 23 - DNA, 1287 bp, synthetic construct
  source 1..1287: mol_type = other DNA
atgaaacatcaccatcaccatcacatgccgtccgttcaagaagtcgaaaaacttctgcatgttctggatcgtaacggcgatggaaaagtatccgccgaggaattgaaggcatttgctgacgattcgaaatgtccgttggatagtaataagatcaaagcattcatcaaagagcatgacaagaacaaggacgggaaattggatttgaaggagttggtgtctattcttagctctcccatgagcgattacgacatccccactactgagaatctttattttcagggcgccatggacgttgtaagcttagataaaccgttcatgtattttgaggaaatcgataacgaactggattatgaaccggaatcggcgaatgaggtggctaaaaaattgccataccagggtcaacttaaattattattgggggaactgttttttttaagtaagcttcagcgtcatgggattcttgacggtgcaacggtggtatacattgggtcggcccctggcactcatatccgctacttacgcgaccacttctacaacttaggcgtcatcatcaaatggatgttgatcgatgggcgccatcacgaccccattcttaatggcttgcgcgatgtaacacttgttacacgtttcgtcgatgaagagtacttacgtagcattaaaaaacaacttcacccatccaaaatcattctgatctctgacgtacgtagcaaacgtgggggtaatgaaccaagtacagctgaccttctgtctaactatgctttacaaaacgtgatgatttccatcttgaatcccgttgcttcatcattgaagtggcgttgccccttcccggaccagtggatcaaagatttctacatccctcacggcaacaagatgttgcaaccgttcgcgccatcctattcggccgaaatgcgcttgttgtccatctacacaggcgagaacatgcgtttaactcgtgtaacaaagtcggatgctgtaaactacgaaaaaaagatgtactatcttaataagattgttcgtaacaaagtggttgtgaatttcgattaccctaaccaggagtacgattattttcatatgtattttatgttgcgtaccgtttattgtaataagacttttccgacaactaaggcaaaggtgctgtttctgcagcaatcgattttccgcttcttaaacatccccaccacaagtacagaaaaagtctcacacgagcccatccaacgcaagatttcttctaaaaatagcatgagcaaaaatcgcaattcaaaacgctcggttcgtagtaacaaatag

SEQ ID NO: 24 - DNA, 3885 bp, synthetic construct
  source 1..3885: mol_type = other DNA
atgaaacatcaccatcaccatcacatgccgtccgttcaagaagtcgaaaaacttctgcatgttctggatcgtaacggcgatggaaaagtatccgccgaggaattgaaggcatttgctgacgattcgaaatgtccgttggatagtaataagatcaaagcattcatcaaagagcatgacaagaacaaggacgggaaattggatttgaaggagttggtgtctattcttagctctcccatgagcgattacgacatccccactactgagaatctttattttcagggcgccatggacgctaatgtcgtctcgtcaagtacgatcgctacctacattgacgccttggcgaaaaatgctagcgagcttgaacagcgcagcactgcttatgagatcaataatgagttagagcttgtatttatcaaaccaccgttgattactctgacgaacgtagttaacatttcaaccattcaagagtcctttattcgttttaccgtcacgaacaaagagggcgttaaaatccgcaccaagatccctcttagtaaagtgcatggattagatgtcaaaaatgttcagctggtagacgccattgataacatcgtatgggagaagaaatcactggttactgaaaaccgcctgcacaaagaatgtctgttgcgtctgtctaccgaggaacgtcatattttccttgactacaagaaatacggttcgagtattcgcttagaacttgtgaacctgatccaggccaagactaaaaattttacaattgattttaaattaaaatacttcttgggaagtggggcacaatccaagtcgtccttgttacatgctatcaaccatcccaaatcccgtcctaacacaagcttggagattgaattcacaccgcgtgataacgaaactgttccttatgacgagctgattaaagaattgacgacgctttctcgccatatcttcatggcctctcccgaaaacgtcatccttagccctccaatcaatgcaccaattaagacatttatgttaccgaagcaagacattgtaggcttggatcttgaaaatctttatgctgttaccaaaacagatggtattccaattacaattcgtgttacatctaacggcctttactgctatttcacccacttgggatatatcatccgttatccggttaagcgtattatcgactctgaggtagtagtttttggcgaagcagtaaaggacaagaattggacagtatacctgattaaattaattgaaccggtaaatgccattaatgatcgcttagaggaaagcaagtatgtcgaatctaaacttgttgatatctgtgatcgcatcgtatttaaaagtaaaaaatacgaagggccttttactacaacaagtgaagttgttgacatgttatctacctatttaccaaaacaacctgaaggtgttattttgttctattccaagggccccaaatcgaacattgactttaagatcaagaaggaaaacactattgatcagaccgccaacgtagtattccgctatatgtccagtgaaccgattattttcggcgagagtagcattttcgttgaatataaaaaattttccaacgacaagggcttcccgaaagagtatgggtcaggcaagattgtattgtataatggtgtcaattatcttaataacatctattgccttgagtatatcaatacgcacaacgaggtgggcattaagtccgtagtagtgcccatcaagttcatcgctgagttcttggtgaacggggagatcttaaaacctcgtatcgacaagactatgaaatatatcaactccgaagactattatgggaatcagcacaacatcattgtagagcacctgcgcgaccagtccattaagattggagatatttttaacgaagataagctgtccgacgtcgggcaccagtacgcgaacaacgataagttccgtttgaatcctgaagtatcatatttcacaaacaaacgcacacgtggtccactggggatcttgtcgaactatgtcaaaacgctgttaatctctatgtattgttcaaagactttccttgatgatagcaacaagcgtaaggtactggctatcgattttggaaacggcgccgacttggagaaatacttttacggcgaaatcgctctgttagttgctacagatccagacgcagatgcgatcgctcgcggtaacgagcgctataataaacttaactccggcattaaaactaagtattacaagttcgactacatccaagagactattcgtagtgatacgttcgtgagttcggtgcgcgaggtattctatttcggtaaatttaatatcatcgactggcagtttgccatccattatagtttccatccacgccactatgccacagttatgaataacttaagtgagcttacagcaagcggtggtaaagtgctgattaccacaatggacggcgataagttgtctaagttgacggacaaaaaaacattcattatccataagaatctgccttcaagtgaaaattatatgtcagtagagaagatcgccgacgaccgcatcgtagtttacaatccgagtaccatgagtactcccatgacggagtacatcatcaaaaagaatgacatcgtccgtgttttcaatgagtacggcttcgtacttgtagataacgtcgattttgcaacgattattgaacgctcaaagaagtttattaatggggcgtcaacaatggaggatcgcccttccactcgcaatttctttgaattaaatcgtggggccatcaagtgcgagggcctggatgtagaagatttattgtcatattacgtcgtgtacgttttctccaagcgttaagctagcaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggacgagattgtaaagaacattcgtgaaggaacgcacgtgttgcttccgttctacgagaccttaccagagttgaacttatcgcttgggaaatccccgttgccttcgcttgagtatggtgcaaactatttcctgcaaattagtcgcgtaaatgatcttaaccgtatgccgacggacatgctgaaattatttacccacgatattatgttacccgaatcagatctggacaaagtatatgagattcttaaaatcaactctgtgaaatactacggccgtagcacgaaagccgacgcggtcgtcgcagacctttctgcgcgcaacaagcttttcaagcgcgagcgcgacgcgattaaatctaacaaccatttgaccgaaaacaacctttacatcagcgactataagatgctgacctttgacgtattccgcccgttgttcgacttcgtgaatgaaaaatattgcattattaaattaccaacgttgtttggccgtggcgtaatcgacaccatgcgtatttactgttccttgtttaaaaatgttcgcttgcttaaatgcgtttctgatagctggttgaaggattctgcgatcatggtcgcttccgatgtatgtaaaaaaaacttggacttgttcatgtcgcatgttaaatcggttactaaatctagctcatggaaggatgtcaattccgttcagttctcaattcttaacaaccccgtagatacggagttcatcaacaaatttttagagttctccaaccgcgtgtatgaggcactgtactacgttcattctttattatactctagcatgaccagtgattcaaaaagcattgaaaacaaacatcagcgccgcctggttaagcttctgttataa

SEQ ID NO: 25 - DNA, 6886 bp, synthetic construct
  source 1..6886: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggcaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctgaaattaatacgactcactataggggaattgtgagcggataacaattcccctctagaaataattttgtttaactttaagaaggagatataccatgaaacatcaccatcaccatcacatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaacccatgagcgattacgacatccccactactgagaatctttattttcagggcgccatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaatagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattccggat

SEQ ID NO: 26 - DNA, 6965 bp, synthetic construct
  source 1..6965: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctgaaattaatacgactcactataggggaattgtgagcggataacaattcccctctagaaataattttgtttaactttaagaaggagatataccatgaaacatcaccatcaccatcacatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaacccatgagcgattacgacatccccactactgagaatctttattttcagggcgccatggacgttgtaagcttagataaaccgttcatgtattttgaggaaatcgataacgaactggattatgaaccggaatcggcgaatgaggtggctaaaaaattgccataccagggtcaacttaaattattattgggggaactgttttttttaagtaagcttcagcgtcatgggattcttgacggtgcaacggtggtatacattgggtcggcccctggcactcatatccgctacttacgcgaccacttctacaacttaggcgtcatcatcaaatggatgttgatcgatgggcgccatcacgaccccattcttaatggcttgcgcgatgtaacacttgttacacgtttcgtcgatgaagagtacttacgtagcattaaaaaacaacttcacccatccaaaatcattctgatctctgacgtacgtagcaaacgtgggggtaatgaaccaagtacagctgaccttctgtctaactatgctttacaaaacgtgatgatttccatcttgaatcccgttgcttcatcattgaagtggcgttgccccttcccggaccagtggatcaaagatttctacatccctcacggcaacaagatgttgcaaccgttcgcgccatcctattcggccgaaatgcgcttgttgtccatctacacaggcgagaacatgcgtttaactcgtgtaacaaagtcggatgctgtaaactacgaaaaaaagatgtactatcttaataagattgttcgtaacaaagtggttgtgaatttcgattaccctaaccaggagtacgattattttcatatgtattttatgttgcgtaccgtttattgtaataagacttttccgacaactaaggcaaaggtgctgtttctgcagcaatcgattttccgcttcttaaacatccccaccacaagtacagaaaaagtctcacacgagcccatccaacgcaagatttcttctaaaaatagcatgagcaaaaatcgcaattcaaaacgctcggttcgtagtaacaaatagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattccggat

SEQ ID NO: 27 - DNA, 2535 bp, synthetic construct
  source 1..2535: mol_type = other DNA
atggacgctaatgtcgtctcgtcaagtacgatcgctacctacattgacgccttggcgaaaaatgctagcgagcttgaacagcgcagcactgcttatgagatcaataatgagttagagcttgtatttatcaaaccaccgttgattactctgacgaacgtagttaacatttcaaccattcaagagtcctttattcgttttaccgtcacgaacaaagagggcgttaaaatccgcaccaagatccctcttagtaaagtgcatggattagatgtcaaaaatgttcagctggtagacgccattgataacatcgtatgggagaagaaatcactggttactgaaaaccgcctgcacaaagaatgtctgttgcgtctgtctaccgaggaacgtcatattttccttgactacaagaaatacggttcgagtattcgcttagaacttgtgaacctgatccaggccaagactaaaaattttacaattgattttaaattaaaatacttcttgggaagtggggcacaatccaagtcgtccttgttacatgctatcaaccatcccaaatcccgtcctaacacaagcttggagattgaattcacaccgcgtgataacgaaactgttccttatgacgagctgattaaagaattgacgacgctttctcgccatatcttcatggcctctcccgaaaacgtcatccttagccctccaatcaatgcaccaattaagacatttatgttaccgaagcaagacattgtaggcttggatcttgaaaatctttatgctgttaccaaaacagatggtattccaattacaattcgtgttacatctaacggcctttactgctatttcacccacttgggatatatcatccgttatccggttaagcgtattatcgactctgaggtagtagtttttggcgaagcagtaaaggacaagaattggacagtatacctgattaaattaattgaaccggtaaatgccattaatgatcgcttagaggaaagcaagtatgtcgaatctaaacttgttgatatctgtgatcgcatcgtatttaaaagtaaaaaatacgaagggccttttactacaacaagtgaagttgttgacatgttatctacctatttaccaaaacaacctgaaggtgttattttgttctattccaagggccccaaatcgaacattgactttaagatcaagaaggaaaacactattgatcagaccgccaacgtagtattccgctatatgtccagtgaaccgattattttcggcgagagtagcattttcgttgaatataaaaaattttccaacgacaagggcttcccgaaagagtatgggtcaggcaagattgtattgtataatggtgtcaattatcttaataacatctattgccttgagtatatcaatacgcacaacgaggtgggcattaagtccgtagtagtgcccatcaagttcatcgctgagttcttggtgaacggggagatcttaaaacctcgtatcgacaagactatgaaatatatcaactccgaagactattatgggaatcagcacaacatcattgtagagcacctgcgcgaccagtccattaagattggagatatttttaacgaagataagctgtccgacgtcgggcaccagtacgcgaacaacgataagttccgtttgaatcctgaagtatcatatttcacaaacaaacgcacacgtggtccactggggatcttgtcgaactatgtcaaaacgctgttaatctctatgtattgttcaaagactttccttgatgatagcaacaagcgtaaggtactggctatcgattttggaaacggcgccgacttggagaaatacttttacggcgaaatcgctctgttagttgctacagatccagacgcagatgcgatcgctcgcggtaacgagcgctataataaacttaactccggcattaaaactaagtattacaagttcgactacatccaagagactattcgtagtgatacgttcgtgagttcggtgcgcgaggtattctatttcggtaaatttaatatcatcgactggcagtttgccatccattatagtttccatccacgccactatgccacagttatgaataacttaagtgagcttacagcaagcggtggtaaagtgctgattaccacaatggacggcgataagttgtctaagttgacggacaaaaaaacattcattatccataagaatctgccttcaagtgaaaattatatgtcagtagagaagatcgccgacgaccgcatcgtagtttacaatccgagtaccatgagtactcccatgacggagtacatcatcaaaaagaatgacatcgtccgtgttttcaatgagtacggcttcgtacttgtagataacgtcgattttgcaacgattattgaacgctcaaagaagtttattaatggggcgtcaacaatggaggatcgcccttccactcgcaatttctttgaattaaatcgtggggccatcaagtgcgagggcctggatgtagaagatttattgtcatattacgtcgtgtacgttttctccaagcgttaa

SEQ ID NO: 28 - DNA, 864 bp, synthetic construct
  source 1..864: mol_type = other DNA
atggacgagattgtaaagaacattcgtgaaggaacgcacgtgttgcttccgttctacgagaccttaccagagttgaacttatcgcttgggaaatccccgttgccttcgcttgagtatggtgcaaactatttcctgcaaattagtcgcgtaaatgatcttaaccgtatgccgacggacatgctgaaattatttacccacgatattatgttacccgaatcagatctggacaaagtatatgagattcttaaaatcaactctgtgaaatactacggccgtagcacgaaagccgacgcggtcgtcgcagacctttctgcgcgcaacaagcttttcaagcgcgagcgcgacgcgattaaatctaacaaccatttgaccgaaaacaacctttacatcagcgactataagatgctgacctttgacgtattccgcccgttgttcgacttcgtgaatgaaaaatattgcattattaaattaccaacgttgtttggccgtggcgtaatcgacaccatgcgtatttactgttccttgtttaaaaatgttcgcttgcttaaatgcgtttctgatagctggttgaaggattctgcgatcatggtcgcttccgatgtatgtaaaaaaaacttggacttgttcatgtcgcatgttaaatcggttactaaatctagctcatggaaggatgtcaattccgttcagttctcaattcttaacaaccccgtagatacggagttcatcaacaaatttttagagttctccaaccgcgtgtatgaggcactgtactacgttcattctttattatactctagcatgaccagtgattcaaaaagcattgaaaacaaacatcagcgccgcctggttaagcttctgttataa

SEQ ID NO: 29 - DNA, 207 bp, Fasciola hepatica
  source 1..207: mol_type = other DNA
atgccgtccgttcaagaagtcgaaaaacttctgcatgttctggatcgtaacggcgatggaaaagtatccgccgaggaattgaaggcatttgctgacgattcgaaatgtccgttggatagtaataagatcaaagcattcatcaaagagcatgacaagaacaaggacgggaaattggatttgaaggagttggtgtctattcttagctct

SEQ ID NO: 30 - DNA, 1002 bp, synthetic construct
  source 1..1002: mol_type = other DNA
atggacgttgtaagcttagataaaccgttcatgtattttgaggaaatcgataacgaactggattatgaaccggaatcggcgaatgaggtggctaaaaaattgccataccagggtcaacttaaattattattgggggaactgttttttttaagtaagcttcagcgtcatgggattcttgacggtgcaacggtggtatacattgggtcggcccctggcactcatatccgctacttacgcgaccacttctacaacttaggcgtcatcatcaaatggatgttgatcgatgggcgccatcacgaccccattcttaatggcttgcgcgatgtaacacttgttacacgtttcgtcgatgaagagtacttacgtagcattaaaaaacaacttcacccatccaaaatcattctgatctctgacgtacgtagcaaacgtgggggtaatgaaccaagtacagctgaccttctgtctaactatgctttacaaaacgtgatgatttccatcttgaatcccgttgcttcatcattgaagtggcgttgccccttcccggaccagtggatcaaagatttctacatccctcacggcaacaagatgttgcaaccgttcgcgccatcctattcggccgaaatgcgcttgttgtccatctacacaggcgagaacatgcgtttaactcgtgtaacaaagtcggatgctgtaaactacgaaaaaaagatgtactatcttaataagattgttcgtaacaaagtggttgtgaatttcgattaccctaaccaggagtacgattattttcatatgtattttatgttgcgtaccgtttattgtaataagacttttccgacaactaaggcaaaggtgctgtttctgcagcaatcgattttccgcttcttaaacatccccaccacaagtacagaaaaagtctcacacgagcccatccaacgcaagatttcttctaaaaatagcatgagcaaaaatcgcaattcaaaacgctcggttcgtagtaacaaatag

SEQ ID NO: 31 - DNA, 1956 bp, synthetic construct
  source 1..1956: mol_type = other DNA
atgcctgaaccccacgccgtcttatacgtaactaacgagttatctcatattgtaaaaaacgggtttcttcccatttggaaacttaccggcgatgagtctttgaatgacctgtggcttgaaaatggaaaatacgccaccgacgtatacgcatacggagatgtatctaaatggacgatccgccagttgcgcggacacgggttcattttcattagcacgcataaaaacgtgcagctggcagacattattaagactgtggacgtccgtatccctcgcgaggtagcacgctcgcatgatatgaaagcattcgaaaatgaaatctcacgtcgccgtatccgcatgcgcaaaggattcggtgacgcgctgcgtaactatgcgtttaaaatggcgattgagtttcatggcagcgaagccgaaacgttgaacgacgccaacccacgtcttcacaagatttacgggatgcccgaaatccctcccttgtacatggagtacgccgagatcggtactcgtttcgatgacgaacccacagatgaaaagttggttagtatgcttgactacattgtttattccgcggaggaggttcattacgtgggctgcggagacttacgcacacttatgcagttcaagaagcgctctcctggccgctttcgccgcgtattgtggcatgtgtacgaccccatcgcgcccgaatgtagcgatcctaatgtaattgtacataacatcatggtcgattccaaaaaagatattcttaaacatatgaacttcttaaaacgtgtggagcgccttttcatttgggatgtcagctcggatcgctctcagatgaacgatcacgagtgggaaacgacgcgtttcgccgaggaccgtttaggtgaggaaatcgcgtacgagatgggcggggctttctcatcggctttgattaagcaccgcatcccaaacagcaaggacgaatatcactgtatttcgacctaccttttcccccaaccgggagcagacgcagacatgtacgagctgcgcaacttcatgcgcttacgtgggtattcacatgtcgaccgtcatatgcacccagacgcttcagtgactaaagtagtgagtcgtgacgttcgcaagatggtggaattgtaccacggccgtgaccgtggtcgtttcttaaagaaacgcttatttgaacatctgcacatcgtccgtaagaatgggctgcttcacgaatcggacgagccacgtgcggacttattctatttgacaaaccgctgcaacatgggtttggagcccagtatctatgaagtgatgaagaaatccgtcattgccaccgcatgggtgggtcgtgcgcctctttatgactatgacgattttgcattgccgcgttcaaccgttatgttaaacggtagttaccgcgatatccgtattttggacgggaacggcgcgatcttatttcttatgtggcgctatcccgatattgtcaagaaggacctgacatatgaccccgcgtgggccatgaattttgcggtcagcttaaaagagcctatcccggatccacctgttccagacatcagtttatgccgctttatcgggttacgcgtcgaatcgagcgttcttcgcgttcgtaatccaacgttgcatgagacggctgacgagttgaaacgtatgggcttagacctttctggccatctgtacgtcaccttgatgagcggggcatatgtgaccgaccttttttggtggtttaagatgattttagattggtctgcgcaaaacaaggagcagaagctgcgtgaccttaagcgctcagcggcggaggtaatcgagtggaaagaacagatggccgaacgtccgtggcacgtccgtaacgaccttatccgcgctctgcgcgaatacaaacgcaagatgggcatgcgtgaaggtgcatcgatcgactcgtggctggaattgcttcgccaccttatgaaacaccaccatcaccatcac

SEQ ID NO: 32 - DNA, 2190 bp, synthetic construct
  source 1..2190: mol_type = other DNA
atgcctgaaccccacgccgtcttatacgtaactaacgagttatctcatattgtaaaaaacgggtttcttcccatttggaaacttaccggcgatgagtctttgaatgacctgtggcttgaaaatggaaaatacgccaccgacgtatacgcatacggagatgtatctaaatggacgatccgccagttgcgcggacacgggttcattttcattagcacgcataaaaacgtgcagctggcagacattattaagactgtggacgtccgtatccctcgcgaggtagcacgctcgcatgatatgaaagcattcgaaaatgaaatctcacgtcgccgtatccgcatgcgcaaaggattcggtgacgcgctgcgtaactatgcgtttaaaatggcgattgagtttcatggcagcgaagccgaaacgttgaacgacgccaacccacgtcttcacaagatttacgggatgcccgaaatccctcccttgtacatggagtacgccgagatcggtactcgtttcgatgacgaacccacagatgaaaagttggttagtatgcttgactacattgtttattccgcggaggaggttcattacgtgggctgcggagacttacgcacacttatgcagttcaagaagcgctctcctggccgctttcgccgcgtattgtggcatgtgtacgaccccatcgcgcccgaatgtagcgatcctaatgtaattgtacataacatcatggtcgattccaaaaaagatattcttaaacatatgaacttcttaaaacgtgtggagcgccttttcatttgggatgtcagctcggatcgctctcagatgaacgatcacgagtgggaaacgacgcgtttcgccgaggaccgtttaggtgaggaaatcgcgtacgagatgggcggggctttctcatcggctttgattaagcaccgcatcccaaacagcaaggacgaatatcactgtatttcgacctaccttttcccccaaccgggagcagacgcagacatgtacgagctgcgcaacttcatgcgcttacgtgggtattcacatgtcgaccgtcatatgcacccagacgcttcagtgactaaagtagtgagtcgtgacgttcgcaagatggtggaattgtaccacggccgtgaccgtggtcgtttcttaaagaaacgcttatttgaacatctgcacatcgtccgtaagaatgggctgcttcacgaatcggacgagccacgtgcggacttattctatttgacaaaccgctgcaacatgggtttggagcccagtatctatgaagtgatgaagaaatccgtcattgccaccgcatgggtgggtcgtgcgcctctttatgactatgacgattttgcattgccgcgttcaaccgttatgttaaacggtagttaccgcgatatccgtattttggacgggaacggcgcgatcttatttcttatgtggcgctatcccgatattgtcaagaaggacctgacatatgaccccgcgtgggccatgaattttgcggtcagcttaaaagagcctatcccggatccacctgttccagacatcagtttatgccgctttatcgggttacgcgtcgaatcgagcgttcttcgcgttcgtaatccaacgttgcatgagacggctgacgagttgaaacgtatgggcttagacctttctggccatctgtacgtcaccttgatgagcggggcatatgtgaccgaccttttttggtggtttaagatgattttagattggtctgcgcaaaacaaggagcagaagctgcgtgaccttaagcgctcagcggcggaggtaatcgagtggaaagaacagatggccgaacgtccgtggcacgtccgtaacgaccttatccgcgctctgcgcgaatacaaacgcaagatgggcatgcgtgaaggtgcatcgatcgactcgtggctggaattgcttcgccaccttatgagcgattacgacatcgacactactatgccctccgtccaggaggtcgaaaaattattacatgtgttagaccgtaacggagatggaaaagttagcgccgaggagctgaaggcattcgcggacgactccaagtgccctcttgactcgaacaagattaaagcatttattaaagaacacgataagaataaagacgggaagttggacttgaaggaattggtatcgatcttatcgagtatgaaacaccaccatcaccatcac

SEQ ID NO: 33 - DNA, 2217 bp, synthetic construct
  source 1..2217: mol_type = other DNA
atgcctgaaccccacgccgtcttatacgtaactaacgagttatctcatattgtaaaaaacgggtttcttcccatttggaaacttaccggcgatgagtctttgaatgacctgtggcttgaaaatggaaaatacgccaccgacgtatacgcatacggagatgtatctaaatggacgatccgccagttgcgcggacacgggttcattttcattagcacgcataaaaacgtgcagctggcagacattattaagactgtggacgtccgtatccctcgcgaggtagcacgctcgcatgatatgaaagcattcgaaaatgaaatctcacgtcgccgtatccgcatgcgcaaaggattcggtgacgcgctgcgtaactatgcgtttaaaatggcgattgagtttcatggcagcgaagccgaaacgttgaacgacgccaacccacgtcttcacaagatttacgggatgcccgaaatccctcccttgtacatggagtacgccgagatcggtactcgtttcgatgacgaacccacagatgaaaagttggttagtatgcttgactacattgtttattccgcggaggaggttcattacgtgggctgcggagacttacgcacacttatgcagttcaagaagcgctctcctggccgctttcgccgcgtattgtggcatgtgtacgaccccatcgcgcccgaatgtagcgatcctaatgtaattgtacataacatcatggtcgattccaaaaaagatattcttaaacatatgaacttcttaaaacgtgtggagcgccttttcatttgggatgtcagctcggatcgctctcagatgaacgatcacgagtgggaaacgacgcgtttcgccgaggaccgtttaggtgaggaaatcgcgtacgagatgggcggggctttctcatcggctttgattaagcaccgcatcccaaacagcaaggacgaatatcactgtatttcgacctaccttttcccccaaccgggagcagacgcagacatgtacgagctgcgcaacttcatgcgcttacgtgggtattcacatgtcgaccgtcatatgcacccagacgcttcagtgactaaagtagtgagtcgtgacgttcgcaagatggtggaattgtaccacggccgtgaccgtggtcgtttcttaaagaaacgcttatttgaacatctgcacatcgtccgtaagaatgggctgcttcacgaatcggacgagccacgtgcggacttattctatttgacaaaccgctgcaacatgggtttggagcccagtatctatgaagtgatgaagaaatccgtcattgccaccgcatgggtgggtcgtgcgcctctttatgactatgacgattttgcattgccgcgttcaaccgttatgttaaacggtagttaccgcgatatccgtattttggacgggaacggcgcgatcttatttcttatgtggcgctatcccgatattgtcaagaaggacctgacatatgaccccgcgtgggccatgaattttgcggtcagcttaaaagagcctatcccggatccacctgttccagacatcagtttatgccgctttatcgggttacgcgtcgaatcgagcgttcttcgcgttcgtaatccaacgttgcatgagacggctgacgagttgaaacgtatgggcttagacctttctggccatctgtacgtcaccttgatgagcggggcatatgtgaccgaccttttttggtggtttaagatgattttagattggtctgcgcaaaacaaggagcagaagctgcgtgaccttaagcgctcagcggcggaggtaatcgagtggaaagaacagatggccgaacgtccgtggcacgtccgtaacgaccttatccgcgctctgcgcgaatacaaacgcaagatgggcatgcgtgaaggtgcatcgatcgactcgtggctggaattgcttcgccaccttcccatgagcgattacgacatccccactactgagaatctttattttcagggcgccatgccctccgtccaggaggtcgaaaaattattacatgtgttagaccgtaacggagatggaaaagttagcgccgaggagctgaaggcattcgcggacgactccaagtgccctcttgactcgaacaagattaaagcatttattaaagaacacgataagaataaagacgggaagttggacttgaaggaattggtatcgatcttatcgagtatgaaacaccaccatcaccatcac

SEQ ID NO: 34 - DNA, 3111 bp, synthetic construct
  source 1..3111: mol_type = other DNA
atgaaacatcaccatcaccatcacatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcccatgagcgattacgacatccccactactgagaatctttattttcagggcgccatgccggagcctcatgctgtgttgtacgttacaaatgaactttcacacattgttaaggatggctttttaccaatctggaaacttaccggcgatgagtcccttaacgatttgtggttagagaacggtaaatacgccacggacgtttatgcctacggtgacgtatcgaagtggaccattcgccagctgcgcggtcatgggtttattttcatctcgacacacaagaacgtccaactcgcggatatcattaagactgtggacgttcggatccctcgtgaggtggcccgctctcatgacatgaaggcgtttgaaaatgaaattgggcgtcgccgtattcggatgcgtaaaggctttggtgatgcattacgtaattacgcctttaagatggcaattgaatttcatggtagcgaggctgaaactctgaacgacgcaaatcctcgtcttcacaaaatctatgggatgccggaaatcccgccactttacatggaatacgcggaaatcgggacacgtttcgatgatgagccgactgacgagaaacttgtatctatgcttgactacattgtctatagcgccgaagaagttcactacatcgggtgcggggacttacggacgcttatgcagtttaaaaaacgttcaccgggtcggtttcggcgtgtgttgtggcatgtatacgacccgattgcaccggagtgctcagatccgaatgttatcgttcataatatcatggttgactccaagaaagacattttgaagcatatgaattttttaaagcgcgttgagcgcttattcatctgggatgtctccagtgaccggagtcagatgaatgatcacgaatgggaaacaacgcgcttcgcagaggatcggctgggtgaggaaatcgcctatgagatggggggtgcgttctcttcggctttaattaagcaccgcatcccaaactcgaaagacgagtatcattgtattagcacttacctgtttccacaaccgggggctgacgcggacatgtacgaattgcgtaattttatgcgtcttcgtgggtactcacacgttgatcgccacatgcatccggatgcttcggtaacaaaggtcgtgtcgcgggatgtgcgtaaaatggtcgagctttaccacggtcgtgatcgcggccggttcctcaaaaaacgcttgtttgagcatttacatatcgtgcgcaagaatgggctcttgcacgaatcggacgagccacgggcggatttattctaccttactaatcggtgtaacatgggtctggagccttcgatttacgaagtcatgaaaaaatcagtcatcgccacggcatgggtcggccgtgcccctctgtatgactatgatgactttgctctcccgcgcagcacagttatgttaaatggcagctaccgcgatatccggattctggatggcaacggcgcaatccttttcctcatgtggcgttatcctgatattgtcaagaaggacttaacatacgacccggcttgggcaatgaacttcgctgtatcacttaaagaaccaattccggatccgcctgtgccggacatcagtttatgtcggtttattggcctccgcgtcgaatcatccgttttgcgcgtacgtaatccaacgcttcacgaaaccgccgatgagttaaaacgtatgggtctggatctgtcgggccatttatacgtgactctcatgagtggtgcttacgtgacagacctcttttggtggttcaaaatgatcctggactggagcgctcaaaatcgggaacagaagcttcgggaccttaaacgctcggccgctgaggttatcgaatggaaggagcagatggcagagcggccatggcatgtacgcaacgacctgattgctgcacttcgcgagtacaagcgtaaaatgggtatgcgggagggggcgtctatcgactcgtggctggagttactgcgccattta

SEQ ID NO: 35 - DNA, 2292 bp, synthetic construct
  source 1..2292: mol_type = other DNA
atgaaacatcaccatcaccatcacggcagcctgcaagaagagaaaccgaaagagggcgttaagaccgagaatgaccacattaacctgaaggtcgctggtcaagatggcagcgtggtgcagtttaagatcaagcgtcacacgccgttgagcaagctgatgaaggcttactgcgagcgtcagggtctgagcatgcgtcagatccgctttcgtttcgatggccagccgatcaatgagactgacaccccagcgcaactggagatggaagatgaagataccatcgacgtctttcagcaacagaccggtggtcccatgagcgattacgacatccccactactgagaatctttattttcagggcgccatgccggagcctcatgctgtgttgtacgttacaaatgaactttcacacattgttaaggatggctttttaccaatctggaaacttaccggcgatgagtcccttaacgatttgtggttagagaacggtaaatacgccacggacgtttatgcctacggtgacgtatcgaagtggaccattcgccagctgcgcggtcatgggtttattttcatctcgacacacaagaacgtccaactcgcggatatcattaagactgtggacgttcggatccctcgtgaggtggcccgctctcatgacatgaaggcgtttgaaaatgaaattgggcgtcgccgtattcggatgcgtaaaggctttggtgatgcattacgtaattacgcctttaagatggcaattgaatttcatggtagcgaggctgaaactctgaacgacgcaaatcctcgtcttcacaaaatctatgggatgccggaaatcccgccactttacatggaatacgcggaaatcgggacacgtttcgatgatgagccgactgacgagaaacttgtatctatgcttgactacattgtctatagcgccgaagaagttcactacatcgggtgcggggacttacggacgcttatgcagtttaaaaaacgttcaccgggtcggtttcggcgtgtgttgtggcatgtatacgacccgattgcaccggagtgctcagatccgaatgttatcgttcataatatcatggttgactccaagaaagacattttgaagcatatgaattttttaaagcgcgttgagcgcttattcatctgggatgtctccagtgaccggagtcagatgaatgatcacgaatgggaaacaacgcgcttcgcagaggatcggctgggtgaggaaatcgcctatgagatggggggtgcgttctcttcggctttaattaagcaccgcatcccaaactcgaaagacgagtatcattgtattagcacttacctgtttccacaaccgggggctgacgcggacatgtacgaattgcgtaattttatgcgtcttcgtgggtactcacacgttgatcgccacatgcatccggatgcttcggtaacaaaggtcgtgtcgcgggatgtgcgtaaaatggtcgagctttaccacggtcgtgatcgcggccggttcctcaaaaaacgcttgtttgagcatttacatatcgtgcgcaagaatgggctcttgcacgaatcggacgagccacgggcggatttattctaccttactaatcggtgtaacatgggtctggagccttcgatttacgaagtcatgaaaaaatcagtcatcgccacggcatgggtcggccgtgcccctctgtatgactatgatgactttgctctcccgcgcagcacagttatgttaaatggcagctaccgcgatatccggattctggatggcaacggcgcaatccttttcctcatgtggcgttatcctgatattgtcaagaaggacttaacatacgacccggcttgggcaatgaacttcgctgtatcacttaaagaaccaattccggatccgcctgtgccggacatcagtttatgtcggtttattggcctccgcgtcgaatcatccgttttgcgcgtacgtaatccaacgcttcacgaaaccgccgatgagttaaaacgtatgggtctggatctgtcgggccatttatacgtgactctcatgagtggtgcttacgtgacagacctcttttggtggttcaaaatgatcctggactggagcgctcaaaatcgggaacagaagcttcgggaccttaaacgctcggccgctgaggttatcgaatggaaggagcagatggcagagcggccatggcatgtacgcaacgacctgattgctgcacttcgcgagtacaagcgtaaaatgggtatgcgggagggggcgtctatcgactcgtggctggagttactgcgccattta

SEQ ID NO: 36 - DNA, 2190 bp, synthetic construct; identical to SEQ ID NO: 32
  source 1..2190: mol_type = other DNA

SEQ ID NO: 37 - DNA, 2163 bp, synthetic construct
  source 1..2163: mol_type = other DNA
atgaaacatcaccatcaccatcacatgccgtccgttcaagaagtcgaaaaacttctgcatgttctggatcgtaacggcgatggaaaagtatccgccgaggaattgaaggcatttgctgacgattcgaaatgtccgttggatagtaataagatcaaagcattcatcaaagagcatgacaagaacaaggacgggaaattggatttgaaggagttggtgtctattcttagctctatgccggagcctcatgctgtgttgtacgttacaaatgaactttcacacattgttaaggatggctttttaccaatctggaaacttaccggcgatgagtcccttaacgatttgtggttagagaacggtaaatacgccacggacgtttatgcctacggtgacgtatcgaagtggaccattcgccagctgcgcggtcatgggtttattttcatctcgacacacaagaacgtccaactcgcggatatcattaagactgtggacgttcggatccctcgtgaggtggcccgctctcatgacatgaaggcgtttgaaaatgaaattgggcgtcgccgtattcggatgcgtaaaggctttggtgatgcattacgtaattacgcctttaagatggcaattgaatttcatggtagcgaggctgaaactctgaacgacgcaaatcctcgtcttcacaaaatctatgggatgccggaaatcccgccactttacatggaatacgcggaaatcgggacacgtttcgatgatgagccgactgacgagaaacttgtatctatgcttgactacattgtctatagcgccgaagaagttcactacatcgggtgcggggacttacggacgcttatgcagtttaaaaaacgttcaccgggtcggtttcggcgtgtgttgtggcatgtatacgacccgattgcaccggagtgctcagatccgaatgttatcgttcataatatcatggttgactccaagaaagacattttgaagcatatgaattttttaaagcgcgttgagcgcttattcatctgggatgtctccagtgaccggagtcagatgaatgatcacgaatgggaaacaacgcgcttcgcagaggatcggctgggtgaggaaatcgcctatgagatggggggtgcgttctcttcggctttaattaagcaccgcatcccaaactcgaaagacgagtatcattgtattagcacttacctgtttccacaaccgggggctgacgcggacatgtacgaattgcgtaattttatgcgtcttcgtgggtactcacacgttgatcgccacatgcatccggatgcttcggtaacaaaggtcgtgtcgcgggatgtgcgtaaaatggtcgagctttaccacggtcgtgatcgcggccggttcctcaaaaaacgcttgtttgagcatttacatatcgtgcgcaagaatgggctcttgcacgaatcggacgagccacgggcggatttattctaccttactaatcggtgtaacatgggtctggagccttcgatttacgaagtcatgaaaaaatcagtcatcgccacggcatgggtcggccgtgcccctctgtatgactatgatgactttgctctcccgcgcagcacagttatgttaaatggcagctaccgcgatatccggattctggatggcaacggcgcaatccttttcctcatgtggcgttatcctgatattgtcaagaaggacttaacatacgacccggcttgggcaatgaacttcgctgtatcacttaaagaaccaattccggatccgcctgtgccggacatcagtttatgtcggtttattggcctccgcgtcgaatcatccgttttgcgcgtacgtaatccaacgcttcacgaaaccgccgatgagttaaaacgtatgggtctggatctgtcgggccatttatacgtgactctcatgagtggtgcttacgtgacagacctcttttggtggttcaaaatgatcctggactggagcgctcaaaatcgggaacagaagcttcgggaccttaaacgctcggccgctgaggttatcgaatggaaggagcagatggcagagcggccatggcatgtacgcaacgacctgattgctgcacttcgcgagtacaagcgtaaaatgggtatgcgggagggggcgtctatcgactcgtggctggagttactgcgccattta

SEQ ID NO: 38 - RNA, 11 nt, synthetic construct
  source 1..11: mol_type = other RNA
gggatcctacc

SEQ ID NO: 39 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 40 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 41 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 42 - DNA, 2076 bp, synthetic construct
  source 1..2076: mol_type = other DNA
atgaagcaaagcactattgcactggcactcttaccgttactgtttacccctgtgacaaaagcaatgaaacatcaccatcaccatcaccccatgagcgattacgacatccccactactgagaatctttattttcagggcgccatgccggagcctcatgctgtgttgtacgttacaaatgaactttcacacattgttaaggatggctttttaccaatctggaaacttaccggcgatgagtcccttaacgatttgtggttagagaacggtaaatacgccacggacgtttatgcctacggtgacgtatcgaagtggaccattcgccagctgcgcggtcatgggtttattttcatctcgacacacaagaacgtccaactcgcggatatcattaagactgtggacgttcggatccctcgtgaggtggcccgctctcatgacatgaaggcgtttgaaaatgaaattgggcgtcgccgtattcggatgcgtaaaggctttggtgatgcattacgtaattacgcctttaagatggcaattgaatttcatggtagcgaggctgaaactctgaacgacgcaaatcctcgtcttcacaaaatctatgggatgccggaaatcccgccactttacatggaatacgcggaaatcgggacacgtttcgatgatgagccgactgacgagaaacttgtatctatgcttgactacattgtctatagcgccgaagaagttcactacatcgggtgcggggacttacggacgcttatgcagtttaaaaaacgttcaccgggtcggtttcggcgtgtgttgtggcatgtatacgacccgattgcaccggagtgctcagatccgaatgttatcgttcataatatcatggttgactccaagaaagacattttgaagcatatgaattttttaaagcgcgttgagcgcttattcatctgggatgtctccagtgaccggagtcagatgaatgatcacgaatgggaaacaacgcgcttcgcagaggatcggctgggtgaggaaatcgcctatgagatggggggtgcgttctcttcggctttaattaagcaccgcatcccaaactcgaaagacgagtatcattgtattagcacttacctgtttccacaaccgggggctgacgcggacatgtacgaattgcgtaattttatgcgtcttcgtgggtactcacacgttgatcgccacatgcatccggatgcttcggtaacaaaggtcgtgtcgcgggatgtgcgtaaaatggtcgagctttaccacggtcgtgatcgcggccggttcctcaaaaaacgcttgtttgagcatttacatatcgtgcgcaagaatgggctcttgcacgaatcggacgagccacgggcggatttattctaccttactaatcggtgtaacatgggtctggagccttcgatttacgaagtcatgaaaaaatcagtcatcgccacggcatgggtcggccgtgcccctctgtatgactatgatgactttgctctcccgcgcagcacagttatgttaaatggcagctaccgcgatatccggattctggatggcaacggcgcaatccttttcctcatgtggcgttatcctgatattgtcaagaaggacttaacatacgacccggcttgggcaatgaacttcgctgtatcacttaaagaaccaattccggatccgcctgtgccggacatcagtttatgtcggtttattggcctccgcgtcgaatcatccgttttgcgcgtacgtaatccaacgcttcacgaaaccgccgatgagttaaaacgtatgggtctggatctgtcgggccatttatacgtgactctcatgagtggtgcttacgtgacagacctcttttggtggttcaaaatgatcctggactggagcgctcaaaatcgggaacagaagcttcgggaccttaaacgctcggccgctgaggttatcgaatggaaggagcagatggcagagcggccatggcatgtacgcaacgacctgattgctgcacttcgcgagtacaagcgtaaaatgggtatgcgggagggggcgtctatcgactcgtggctggagttactgcgccatttataa

SEQ ID NO: 43 - protein, 691 aa, synthetic construct
MKQSTIALALLPLLFTPVTKAMKHHHHHHPMSDYDIPTTENLYFQGAMPEPHAVLYVTNELSHIVKDGFLPIWKLTGDESLNDLWLENGKYATDVYAYGDVSKWTIRQLRGHGFIFISTHKNVQLADIIKTVDVRIPREVARSHDMKAFENEIGRRRIRMRKGFGDALRNYAFKMAIEFHGSEAETLNDANPRLHKIYGMPEIPPLYMEYAEIGTRFDDEPTDEKLVSMLDYIVYSAEEVHYIGCGDLRTLMQFKKRSPGRFRRVLWHVYDPIAPECSDPNVIVHNIMVDSKKDILKHMNFLKRVERLFIWDVSSDRSQMNDHEWETTRFAEDRLGEEIAYEMGGAFSSALIKHRIPNSKDEYHCISTYLFPQPGADADMYELRNFMRLRGYSHVDRHMHPDASVTKVVSRDVRKMVELYHGRDRGRFLKKRLFEHLHIVRKNGLLHESDEPRADLFYLTNRCNMGLEPSIYEVMKKSVIATAWVGRAPLYDYDDFALPRSTVMLNGSYRDIRILDGNGAILFLMWRYPDIVKKDLTYDPAWAMNFAVSLKEPIPDPPVPDISLCRFIGLRVESSVLRVRNPTLHETADELKRMGLDLSGHLYVTLMSGAYVTDLFWWFKMILDWSAQNREQKLRDLKRSAAEVIEWKEQMAERPWHVRNDLIAALREYKRKMGMREGASIDSWLELLRHL